SEQUENCE LISTING (21 sequences)

SEQ ID NO: 1 - protein, 695 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTEQKLISEEDLEMETSQFPGEEKPQASPEGRPESETSCAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 2 - protein, 695 aa, Homo sapiens
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLTEQKLISEEDLEMETSQFPGEEKPQASPEGRPESETSCAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 695 aa, Homo sapiens
APTSSSTKKTQLQLEHLRLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCHSIISTLTEQKLISEEDLEMETSQFPGEEKPQASPEGRPESETSCAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 4 - protein, 133 aa, Homo sapiens [sequence omitted]

SEQ ID NO: 5 - protein, 133 aa, Homo sapiens
APTSSSTKKTQLQLEHLLLDLQMILNGINNYKNPKLTDMLTFEFYMPKKATELKHLQCLERELKPLEEVLNLAQSKNFHLRPRDLISNINVIVLELKGSETTFMCEYADETATIVEFLNRWITFCQSIISTLT

SEQ ID NO: 6 - protein, 133 aa, Homo sapiens
APTSSSTKKTQLQLEHLRLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCHSIISTLT

SEQ ID NO: 7 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 8 - protein, 22 aa, Homo sapiens
MLLLVTSLLLCELPHPAFLLIP

SEQ ID NO: 9 - protein, 37 aa, synthetic construct
EQKLISEEDLEMETSQFPGEEKPQASPEGRPESETSC

SEQ ID NO: 10 - protein, 21 aa, synthetic construct
SGATNFSLLKQAGDVEENPGP

SEQ ID NO: 11 - protein, 695 aa, Homo sapiens
APTSSSTKKTQLQLEHLRLDLEMILNGINNYKNPKLTDMLTFEFYMPKKATELKHLQCLERELKPLEEVLNLAQSKNFHLRPRDLISRINVIVLELKGSETTFMCEYADETATIVEFLNRWITFCHSIISTLTEQKLISEEDLEMETSQFPGEEKPQASPEGRPESETSCAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 12 - protein, 133 aa, Homo sapiens
APTSSSTKKTQLQLEHLRLDLEMILNGINNYKNPKLTDMLTFEFYMPKKATELKHLQCLERELKPLEEVLNLAQSKNFHLRPRDLISRINVIVLELKGSETTFMCEYADETATIVEFLNRWITFCHSIISTLT

SEQ ID NO: 13 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 14 - protein, 25 aa, Homo sapiens
IPWLGHLLVGLSGAFGFIILVYLLI

SEQ ID NO: 15 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 16 - protein, 5 aa, synthetic construct
GGGGS

SEQ ID NO: 17 - protein, 15 aa, synthetic construct
GGGGSGGGGSGGGGS

SEQ ID NO: 18 - protein, 26 aa, synthetic construct
SGGGSGGGGSGGGGSGGGGSGGGSLQ

SEQ ID NO: 19 - protein, 10 aa, synthetic construct
GGGGSGGGGS

SEQ ID NO: 20 - protein, 5 aa, synthetic construct
  VARIANT 1..5: note = Can be present in repeats of any integer
GGGGS

SEQ ID NO: 21 - protein, 5 aa, synthetic construct
GSGPR

(19) United States
(12) Patent Application Publication
(10) Pub. No.: US 2025/0333697 A1
(43) Pub. Date: Oct. 30, 2025

(54) TETHERED INTERLEUKIN-2 RECOMBINANT RECEPTORS AND METHODS OF USE

(71) Applicant: Sonoma Biotherapeutics, Inc., South San Francisco, CA (US)

(72) Inventors: Eleonora TROTTA, San Mateo, CA (US); Frederic VAN GOOL, San Francisco, CA (US); James MATTHAEI, Seattle, WA (US)

(73) Assignee: Sonoma Biotherapeutics, Inc., South San Francisco, CA (US)

(21) Appl. No.: 19/106,603

(22) PCT Filed: Aug. 24, 2023
(86) PCT No.: PCT/US2023/072860

§ 371 Date: Feb. 25, 2025

Related U.S. Application Data
(60) Provisional application No. 63/373,591, filed on Aug. 26, 2022.
(60) Provisional application No. 63/498,803, filed on Apr. 27, 2023.
(60) Provisional application No. 63/499,954, filed on May 3, 2023.

(51) Int. Cl. C12N 5/0783; A61K 35/17; A61K 40/11; A61K 40/31; A61P 37/02; C07K 14/55; C07K 14/705; C07K 14/71; C07K 14/715; C12N 15/86

(52) U.S. Cl. CPC C12N 5/0637; A61K 35/17; A61K 40/11; A61K 40/31; A61P 37/02; C07K 14/55; C07K 14/70521; C07K 14/71; C07K 14/7155; C12N 15/86; A61K 2239/15; C07K 2319/03; C07K 2319/033; C12N 2510/00; C12N 2740/15043

(57) ABSTRACT

Provided are recombinant cytokine receptors that comprise an IL-2 receptor polypeptide that is tethered to IL-2 cytokine. Also provided herein are Treg cells comprising recombinant cytokine receptors and methods of use.

### CROSS-REFERENCE TO RELATED APPLICATIONS

This application claims the benefit of, and priority to, U.S. Provisional Application 63/373,591, filed on Aug. 26, 2022, U.S. Provisional Application 63/498,803, filed on Apr. 27, 2023, and U.S. Provisional Application 63/499,954, filed on May 3, 2023, the contents of each of which are hereby incorporated herein by reference in their entirety.

### REFERENCE TO AN ELECTRONIC SEQUENCE LISTING

The contents of the electronic sequence listing (237752000541SEQLIST.xml; Size: 23,881 bytes; and Date of Creation: Aug. 18, 2023) are herein incorporated by reference in their entirety.

### BACKGROUND

The immune system plays a critical role in maintaining organismal homeostasis, poised between the elimination of foreign antigens and the self-tolerance of autoantigens. In particular, hyperactive immune dysregulation can lead to various autoimmune disorders (e.g., irritable bowel syndrome, systemic lupus erythematosus, alopecia areata, multiple sclerosis), which is often the result of overactive effector T lymphocytes or underactive regulatory T lymphocytes (i.e., Tregs). Current therapies for autoimmune diseases involve the administration of steroids, which can cause serious side-effects in patients and often offer little relief to patients.

Regulatory T cells are a key player in the maintenance of organismal homeostasis to prevent the destruction of otherwise healthy tissues. Tregs are a unique subset of T cells that inhibit the cytotoxic or pro-inflammatory activity of effector CD4+ or effector CD8+ T cells. Tregs differentiate from the parent T lymphocyte lineage upon the upregulation of key Treg genes, in particular IL-2Rα and FOXP3 (see, e.g., Chen, M L et al. (2005), Proc Natl Accad Sci USA; 102(2):419-424 and Liu, V C et al. (2007), J Immunol; 178(5):2883-2892, hereby incorporated by reference in their entirety). Upon T cell receptor (TCR) activation, these Tregs are responsible for directly suppressing effector T cell activity via cytokine production, e.g., TGF-β and IL-10 (see, e.g., Chen, J et al. (2019), Trends Mol Med; 25(11):1010-1023, hereby incorporated by reference in its entirety), and the engagement of immune checkpoint receptors, e.g., TIGIT- or CTLA-4-engagement (see, e.g., Knochelmann, H M et al. (2018), Cell Mol Immunol; 15(5):458-469, hereby incorporated by reference in its entirety). While effector T cells are able to produce the cytokine IL-2 upon TCR activation to support their own expansion, Treg cells are dependent upon exogenous IL-2 to promote Treg survival and maintenance, as Tregs are unable to produce their own IL-2. However, Treg cells require IL-2 signaling for survival and proliferation, like all lineages of T lymphocytes. This biological mechanism ensures that Tregs are maintained in tissue niches that are enriched with active effector T cells, thus creating a cellular negative feedback mechanism whereby the increased IL-2 production by the target effector T cell promotes the expansion of the Tregs that then act to inhibit effector T cell activity and thus downstream inhibit their own expansion and survival (see, e.g., Shevyrev, D & Tereshchenko, V (2020), Front Immunol; 10:3100, 1-13). Thus, targeting of the IL-2 receptor signaling pathway in Tregs is one area of recent interest in the effort to identify novel therapies for autoimmune disorders.

### SUMMARY

In one aspect, the present application relates to recombinant cytokine receptors comprising an interleukin-2 (IL-2) cytokine molecule tethered to the extracellular domain of the IL-2Rβ. Also provided herein are cells that express the IL-2 tethered IL-2Rβ recombinant cytokine receptors and methods of use.

In one aspect of the present invention, there is provided a regulatory T cell (Treg) comprising a recombinant cytokine receptor, wherein the recombinant cytokine receptor comprises: an IL-2 cytokine; an IL-2 receptor beta extracellular domain; a transmembrane domain; and an IL-2 receptor beta intracellular domain: wherein the IL-2 receptor beta extracellular domain is tethered to the IL-2 cytokine by a polypeptide linker. In some embodiments, the recombinant cytokine receptor forms a protein complex with IL-2Rγ. In some embodiments, the recombinant cytokine receptor engages in IL-2 signaling in the absence of exogenous IL-2.

In some embodiments according to any of the Tregs described above, the IL-2 cytokine comprises at least one amino acid substitution that reduces affinity for IL-2Rα and/or IL-2Rγ by at least about two-fold. In some embodiments, the IL-2 cytokine comprises one or more amino acid substitutions selected from the amino acid positions 18, 22, 126, 38, 43, 61, 15, 16, 19, 20, 22, 23, and 81. In some embodiments, the IL-2 cytokine comprises one or more amino acid substitutions selected from the group consisting of L18R, Q22E, Q126H, R38D, K43E, E61R, E15S, H16Q, L19V, D20L, Q22K, M23Q, M23A, and R81D. In some embodiments, the IL-2 cytokine comprises the amino acid substitutions L18R, Q22E, and Q126H; and/or R38D, K43E, and E61R; and/or E15S, H16Q, L19V, D20L, M23Q, and R81D; and/or E15S, H16Q, L19V, D20L, Q22K, and M23A. In some embodiments, the Treg comprises a recombinant cytokine receptor comprising: a. a WT IL-2 cytokine, a polypeptide linker, an IL-2Rβ extracellular domain, an IL-2Rβ transmembrane domain, and an IL-2Rβ intracellular domain: b. a 3×IL-2 cytokine, a polypeptide linker, an IL-2Rβ extracellular domain, an IL-2Rβ transmembrane domain, and an IL-2Rβ intracellular domain; c. an REH IL-2 cytokine, a polypeptide linker, an IL-2Rβ extracellular domain, an IL-2Rβ transmembrane domain, and an IL-2Rβ intracellular domain; d. a 3×REH IL-2 cytokine, a polypeptide linker, an IL-2Rβ extracellular domain, an IL-2Rβ transmembrane domain, and an IL-2Rβ intracellular domain; e. an IL-2 cytokine comprising substitutions at positions E15S, H16Q, L19V, D20L, M23Q, and R81D, a polypeptide linker, an IL-2Rβ extracellular domain comprising substitutions at positions H133D and Y134F, an IL-2Rβ transmembrane domain, and an IL-2Rβ intracellular domain; or f. an IL-2 cytokine comprising substitutions at positions E15S, H16Q, L19V, D20L, Q22K, and M23A, a polypeptide linker, an IL-2Rβ extracellular domain comprising substitutions at positions H133D and Y134F, an IL-2Rβ transmembrane domain, and an IL-2Rβ intracellular domain. In some embodiments, the polypeptide linker comprises glycines and serines.

In some embodiments according to any of the Tregs described above, the Treg comprises a recombinant cytokine receptor comprising: a. an IL-2 cytokine comprising the amino acid sequence of SEQ ID NO:4, a polypeptide linker, an IL-2Rβ extracellular domain, an IL-2Rβ transmembrane domain, and an IL-2Rβ intracellular domain; b. an IL-2 cytokine comprising the amino acid sequence of SEQ ID NO:5, a polypeptide linker, an IL-2Rβ extracellular domain, an IL-2Rβ transmembrane domain, and an IL-2Rβ intracellular domain; c. an IL-2 cytokine comprising the amino acid sequence of SEQ ID NO:6, a polypeptide linker, an IL-2Rβ extracellular domain, an IL-2Rβ transmembrane domain, and an IL-2Rβ intracellular domain; or d. an IL-2 cytokine comprising the amino acid sequence of SEQ ID NO:12, a polypeptide linker, an IL-2Rβ extracellular domain, an IL-2Rβ transmembrane domain, and an IL-2Rβ intracellular domain.

In some embodiments according to any of the Tregs described above, the Treg comprises a recombinant cytokine receptor comprising: a. an IL-2 cytokine comprising an amino acid sequence comprising at least 80%, at least 85%, at least 90%, at least 95%, at least 96%, at least 97%, at least 98%, at least 99% or 100% sequence identity with the amino acid sequence selected from the group consisting of SEQ ID NOs:4-6 and 12; b. a polypeptide linker comprising an amino acid sequence selected from the group consisting of SEQ ID NOs:9 and 16-21; c. an IL-2 receptor beta extracellular domain comprising at least 80%, at least 85%, at least 90%, at least 95%, at least 96%, at least 97%, at least 98%, at least 99% or 100% sequence identity with the amino acid sequence set forth in SEQ ID NO:13; d. an IL-2 receptor beta transmembrane domain comprising at least 80%, at least 85%, at least 90%, at least 95%, at least 96%, at least 97%, at least 98%, at least 99% or 100% sequence identity with the amino acid sequence set forth in SEQ ID NO:14; and/or e. an IL-2 receptor beta intracellular domain comprises at least 80%, at least 85%, at least 90%, at least 95%, at least 96%, at least 97%, at least 98%, at least 99% or 100% sequence identity with the amino acid sequence set forth in SEQ ID NO:15. In some embodiments, the IL-2 cytokine comprises an amino acid sequence comprising at least 80%, at least 85%, at least 90%, at least 95%, at least 96%, at least 97%, at least 98%, at least 99% or 100% sequence identity with the amino acid sequence set forth in SEQ ID NO:4. In some embodiments, the recombinant cytokine receptor does not comprise a T cell receptor activation domain or a T cell costimulatory domain, optionally wherein the T cell receptor activation domain or T cell costimulatory domain is a CD28 signaling domain.

In some embodiments according to any of the Tregs described above, the Treg comprises a recombinant cytokine receptor comprising: a. an IL-2 cytokine comprising the amino acid sequence of SEQ ID NO:4, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15; b. an IL-2 cytokine comprising the amino acid sequence of SEQ ID NO:5, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15; c. an IL-2 cytokine comprising the amino acid sequence of SEQ ID NO:6, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15; or d. an IL-2 cytokine comprising the amino acid sequence of SEQ ID NO:12, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15. In some embodiments, the recombinant cytokine receptor comprises an amino acid sequence comprising at least 80%, at least 85%, at least 90%, at least 95%, at least 96%, at least 97%, at least 98%, at least 99% or 100% sequence identity with the amino acid sequence selected from the group consisting of SEQ ID NOs:1-3 and 11.

In some embodiments according to any of the Tregs described above, the Treg comprises a recombinant cytokine receptor consisting of: an IL-2 cytokine; an IL-2 receptor beta extracellular domain; a transmembrane domain; and an IL-2 receptor beta intracellular domain; wherein the IL-2 receptor beta extracellular domain is tethered to the IL-2 cytokine thereof by a polypeptide linker.

In some embodiments according to any of the Tregs described above, the Treg is CD25+ and expresses FOXP3 and/or HELIOS. In some embodiments, the Treg further comprising a chimeric antigen receptor (CAR).

In another aspect of the present invention, there is provided a recombinant cytokine receptor comprises an IL-2 cytokine, an IL-2 receptor beta extracellular domain, a transmembrane domain, and an IL-2 receptor beta intracellular domain, wherein the IL-2 receptor beta extracellular domain is tethered to the IL-2 cytokine by a polypeptide linker. In some embodiments, the recombinant cytokine receptor does not include a T cell receptor activation domain or a T cell costimulatory domain. In some embodiments, the IL-2 receptor extracellular domain comprises the extracellular domain of IL-2Rβ. In some embodiments, the polypeptide linker comprises glycines and serines. In some embodiments, the polypeptide linker comprises an amino acid sequence selected from the group consisting of SEQ ID NOs:9 and 16-21. In some embodiments, the recombinant cytokine receptor can form a protein complex with IL-2Rγ.

In some embodiments, the cytokine receptor engages in IL-2 signaling in the absence of exogenous IL-2.

In some embodiments according to any of the recombinant cytokine receptors described above, the IL-2 cytokine comprises at least one amino acid substitution that reduces affinity for IL-2Rα and/or IL-2Rγ by at least about two-fold. In some embodiments, the IL-2 cytokine comprises one or more amino acid substitutions selected from the amino acid positions 18, 22, 126, 38, 43, 61, 15, 16, 19, 20, 22, 23, and 81. In some embodiments, the IL-2 cytokine comprises one or more amino acid substitutions selected from the group consisting of L18R, Q22E, Q126H, R38D, K43E, E61R, E15S, H16Q, L19V, D20L, Q22K, M23Q, M23A, and R81D. In some embodiments, the IL-2 cytokine comprises the amino acid substitutions L18R, Q22E, and Q126H; and/or R38D, K43E, and E61R. In some embodiments, the IL-2 cytokine comprises the amino acid substitutions L18R, Q22E, and Q126H; and/or R38D, K43E, and E61R; and/or E15S, H16Q, L19V, D20L, M23Q, and R81D; and/or E15S, H16Q, L19V, D20L, Q22K, and M23A. In some embodiments, the recombinant cytokine receptor comprises: a. a WT IL-2 cytokine, a polypeptide linker, an IL-2Rβ extracellular domain, an IL-2Rβ transmembrane domain, and an IL-2Rβ intracellular domain; b. a 3×IL-2 cytokine, a polypeptide linker, an IL-2Rβ extracellular domain, an IL-2Rβ transmembrane domain, and an IL-2Rβ intracellular domain; c. an REH IL-2 cytokine, a polypeptide linker, an IL-2Rβ extracellular domain, an IL-2Rβ transmembrane domain, and an IL-2Rβ intracellular domain; d. a 3×REH IL-2 cytokine, a polypeptide linker, an IL-2Rβ extracellular domain, an IL-2Rβ transmembrane domain, and an IL-2Rβ intracellular domain; e. an IL-2 cytokine comprising substitutions at positions E15S, H16Q, L19V, D20L, M23Q, and R81D, a polypeptide linker, an IL-2Rβ extracellular domain comprising substitutions at positions H133D and Y134F, an IL-2Rβ transmembrane domain, and an IL-2Rβ intracellular domain; or f. an IL-2 cytokine comprising substitutions at positions E15S, H16Q, L19V, D20L, Q22K, and M23A, a polypeptide linker, an IL-2Rβ extracellular domain comprising substitutions at positions H133D and Y134F, an IL-2Rβ transmembrane domain, and an IL-2Rβ intracellular domain.

In some embodiments according to any of the recombinant cytokine receptors described above, the IL-2 cytokine comprises an amino acid sequence comprising at least about 80%, at least about 85%, at least about 90%, at least about 95%, at least about 96%, at least about 97%, at least about 98%, at least about 99% or about 100% sequence identity with the amino acid sequence selected from the group consisting of SEQ ID NOs:4-6 and 12. In some embodiments, the IL-2 cytokine comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:4. In some embodiments, the recombinant cytokine receptor does not comprise a T cell receptor activation domain or a T cell costimulatory domain, optionally wherein the T cell receptor activation domain or T cell costimulatory domain is a CD28 signaling domain. In some embodiments, the IL-2 cytokine comprises one or more amino acid substitutions selected from the group consisting of F42A, F42K, R38D, R38A, E61R, R38D and E61R, K35D, K43E, K43D, E15S, H16Q, L19V, D20L, Q22K, M23Q, M23A, and R81D. In some embodiments, the IL-2 cytokine comprises the amino acid substitutions R38D, E61R, and K43E. In some embodiments, the IL-2 cytokine comprises the amino acid substitutions R38A, E61R, and K43E. In some embodiments, the IL-2 cytokine comprises the amino acid substitutions R38D, E61R, and K43D. In some embodiments, the IL-2 cytokine comprises the amino acid substitutions R38A, E61R, and K43D. In some embodiments, the IL-2 receptor comprises an IL-2 cytokine comprising one or more of the amino acid substitutions selected from the group consisting of L18R, Q22E, Q126K, Q126H, Q126M, and Q126R. In some embodiments the IL-2 cytokine comprises the amino acid substitutions L18R, Q22E, and Q126K. In some embodiments the IL-2 cytokine comprises the amino acid substitutions L18R, Q22E, and Q126M. In some embodiments the IL-2 cytokine comprises the amino acid substitutions L18R, Q22E, and Q126R. In some embodiments, the recombinant cytokine receptor comprises: a. an IL-2 cytokine comprising the amino acid sequence of SEQ ID NO:4, a polypeptide linker, an IL-2Rβ extracellular domain, an IL-2Rβ transmembrane domain, and an IL-2Rβ intracellular domain; b. an IL-2 cytokine comprising the amino acid sequence of SEQ ID NO:5, a polypeptide linker, an IL-2Rβ extracellular domain, an IL-2Rβ transmembrane domain, and an IL-2Rβ intracellular domain; c. an IL-2 cytokine comprising the amino acid sequence of SEQ ID NO:6, a polypeptide linker, an IL-2Rβ extracellular domain, an IL-2Rβ transmembrane domain, and an IL-2Rβ intracellular domain; or d. an IL-2 cytokine comprising the amino acid sequence of SEQ ID NO:12, a polypeptide linker, an IL-2Rβ extracellular domain, an IL-2Rβ transmembrane domain, and an IL-2Rβ intracellular domain.

In some embodiments according to any of the recombinant cytokine receptors described above, the recombinant cytokine receptor comprises an IL-2 receptor beta extracellular domain comprising at least 80%, at least 85%, at least 90%, at least 95%, at least 96%, at least 97%, at least 98%, at least 99% or 100% sequence identity with the amino acid sequence set forth in SEQ ID NO:13. In some embodiments, the recombinant cytokine receptor comprises an IL-2 receptor beta transmembrane domain comprising at least 80%, at least 85%, at least 90%, at least 95%, at least 96%, at least 97%, at least 98%, at least 99% or 100% sequence identity with the amino acid sequence set forth in SEQ ID NO:14. In some embodiments, the recombinant cytokine receptor comprises an IL-2 receptor beta intracellular domain comprising at least 80%, at least 85%, at least 90%, at least 95%, at least 96%, at least 97%, at least 98%, at least 99% or 100% sequence identity with the amino acid sequence set forth in SEQ ID NO:15. In some embodiments, the recombinant cytokine receptor comprises an IL-2 receptor beta polypeptide comprising at least 80%, at least 85%, at least 90%, at least 95%, at least 96%, at least 97%, at least 98%, at least 99% or 100% sequence identity with the amino acid sequence set forth in SEQ ID NO:7.

In some embodiments according to any of the recombinant cytokine receptors described above, the recombinant cytokine receptor comprises: a. an IL-2 cytokine comprising the amino acid sequence of SEQ ID NO:4, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15; b. an IL-2 cytokine comprising the amino acid sequence of SEQ ID NO:5, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15; c. an IL-2 cytokine comprising the amino acid sequence of SEQ ID NO:6, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15; or d. an IL-2 cytokine comprising the amino acid sequence of SEQ ID NO:12, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15. In some embodiments, the recombinant cytokine receptor comprises an amino acid sequence comprising at least about 80%, at least about 85%, at least about 90%, at least about 95%, at least about 96%, at least about 97%, at least about 98%, at least about 99% or about 100% sequence identity with the amino acid sequence selected from the group consisting of SEQ ID NOs:1-3 and 11.

In some embodiments according to any of the recombinant cytokine receptors described above, the recombinant cytokine receptor consists of an IL-2 cytokine, an IL-2 receptor beta extracellular domain, a transmembrane domain, and an IL-2 receptor beta intracellular domain, wherein the IL-2 receptor beta extracellular domain is tethered to the IL-2 cytokine by a polypeptide linker.

Also provided herein is a nucleic acid encoding an IL-2-tethered IL-2Rβ recombinant cytokine receptor. Also provided herein is a nucleic acid encoding any of the recombinant cytokine receptors described herein. Also provided herein is a vector comprising the nucleic acid provided herein. In some embodiments, the vector is a lentiviral vector. In some embodiments, the vector further comprises a marker gene. In some embodiments, the marker gene is a transmembrane protein. In some embodiments, the transmembrane protein is EGFR.

Also provided herein is a T cell comprising any of the recombinant cytokine receptors described herein, a nucleic acid encoding the recombinant cytokine receptor, or a vector comprising the nucleic acid. In some embodiments, the T cell is a regulatory T cell (Treg), wherein the Treg is CD25+ and expresses FOXP3 and/or HELIOS. In some embodiments, the T cell, such as Treg cell, further comprises a chimeric antigen receptor (CAR). Also provided herein is a composition comprising the nucleic acid that encodes any of the recombinant cytokine receptors described herein, a vector comprising the nucleic acid encoding the recombinant cytokine receptor(s), or a T cell, such as a Treg cell, that expresses the recombinant cytokine receptor(s).

In another aspect of the present invention, there is provided a method of treating an immune related disorder comprising administering a T cell, such as a Treg cell (e.g., any of the Treg cells described herein), that expresses any of the recombinant cytokine receptors described herein or a composition comprising the T cell, to an individual in need thereof. In some embodiments, the cells are autologous to the individual. In some embodiments, the individual is human. In some embodiments, the individual expresses IL-2. Also provided herein is use of a T cell, such as a Treg, comprising the recombinant cytokine receptor provided herein for treating an immune related disorder in an individual. In some embodiments, the Treg prevents, ameliorates, or cures an immune-related disorder.

In another aspect of the present invention, there is provided a method of expanding a transduced Treg cell in the absence of exogenous IL-2 comprising introducing a nucleic acid that encodes an IL-2-tethered IL-2Rβ recombinant cytokine receptor or a vector comprising the nucleic acid into the Treg cell and culturing the cell.

In some embodiments according to any of the recombinant cytokine receptors described above, the recombinant cytokine receptor does not activate IL-2 signaling on a cell that does not comprise the recombinant cytokine receptor. In some embodiments, the recombinant cytokine receptor does not activate IL-2 signaling on Tregs that do not comprise the recombinant cytokine receptor. In some embodiments, the recombinant cytokine receptor does not activate signaling of an IL-2 receptor comprising a different amino acid sequence.

In some embodiments according to any of the recombinant cytokine receptors described above, the recombinant cytokine receptor does not cause the transduced Treg cell to secrete one or more cytokines more than a Treg cell cultured with exogenous IL-2 cytokine. In some embodiments, the recombinant cytokine receptor does not cause the transduced Treg cell to secrete one or more cytokines at a level higher than the level(s) of cytokine secretion(s) from a Treg cell cultured with exogenous IL-2 cytokine.

In some embodiments according to any of the Tregs described above, at least one Treg marker selected from the group consisting of CD4+, CD25+, and CD127lo is detected in a Treg cell that comprises the recombinant cytokine receptor. In some embodiments, FOXP3 and/or HELIOS levels are detected in a Treg cell that comprises the recombinant cytokine receptor. In some embodiments, the methylation pattern of Treg-specific differentially regulated genes is detected in a Treg cell that comprises the recombinant cytokine receptor. In some embodiments, the Treg-specific differentially regulated gene is FOXP3.

In some embodiments according to any of the Tregs described above, the Treg cell comprising the recombinant cytokine receptor is able to proliferate in the absence of IL-2.

In some embodiments according to any of the Tregs described above, one or more markers of endogenous IL-2 signaling is detected in a Treg cell that comprises the recombinant cytokine receptor. In some embodiments, the one or more markers of endogenous IL-2 signaling comprises phosphorylated STAT5.

In some embodiments according to any of the Tregs described above, the suppressive activity of the Treg on CD8+ and/or CD4+ T cells is increased. In some embodiments, the suppressive activity of the Treg on CD8+ and/or CD4+ T cells is increased compared to a Treg cell that does not comprise the recombinant cytokine receptor. In some embodiments, the suppressive activity comprises a decreased CD4+ and/or CD8+ T cell rate of division.

In some embodiments according to any of the Tregs described above, the relative number of Treg cells in a composition comprising a population of Treg cells transduced with the recombinant cytokine receptor increases over time.

In some embodiments according to any of the Tregs described above, a composition comprising a population of Treg cells transduced with the recombinant cytokine receptor grown without IL-2 contains a similar number of viable cells compared to a composition comprising a population of the same Treg cells not transduced with the recombinant cytokine receptor grown with IL-2.

In some embodiments according to any of the Tregs described above, at least about 80% of the cells in the population of Treg cells transduced with the recombinant cytokine maintain FOXP3 and/or HELIOS expression about 14 days after transduction. In some embodiments, at least about 80% of the cells in the population of Treg cells transduced with the recombinant cytokine maintain FOXP3 and/or HELIOS expression about 23 days after transduction.

In some embodiments according to any of the Tregs described above, the Treg cells comprise a recombinant cytokine receptor, wherein: i. the in vitro and/or in vivo suppressive activity of the Treg on CD8+ and/or CD4+ T cells is increased compared to a control Treg that is not transduced with the recombinant cytokine receptor; ii. the rate of division of CD4+ and/or CD8+ T cells is decreased when cultured in the presence of the Treg compared to the rate of division of CD4+ and/or CD8+ T cells when cultured without the Treg; iii. the relative amount of Treg cells in a composition comprising a population of Treg cells transduced with a recombinant cytokine receptor increases over time; iv. a composition comprising a population of Treg cells transduced with the recombinant cytokine receptor grown without IL-2 contains a similar number of viable cells compared to a composition comprising a population of the same Treg cells not transduced with the recombinant cytokine receptor grown with IL-2; v. at least 80% of the cells in the population of Treg cells transduced with the recombinant cytokine receptor maintain FOXP3 and/or HELIOS expression about 14 days after transduction; vi. at least 80% of the cells in the population of Treg cells transduced with the recombinant cytokine receptor maintain FOXP3 and/or HELIOS expression about 23 days after transduction; vii. the population of Treg cells transduced with the recombinant cytokine receptor expands at least two-fold more than a population of the same Treg cells not transduced with the recombinant cytokine receptor; viii. the IL-10 cytokine levels produced by the Treg in vitro and/or in vivo are equivalent or increased compared to a control Treg that is not transduced with the recombinant cytokine receptor; ix. the IFN-γ cytokine levels produced by the Treg in vitro and/or in vivo are equivalent or increased compared to a control Treg that is not transduced with the recombinant cytokine receptor; x. the Gr-B cytokine levels produced by the Treg in vitro and/or in vivo are equivalent or increased compared to a control Treg that is not transduced with the recombinant cytokine receptor; xi. the Treg functionally re-activates to higher levels than control Tregs that are not transduced with the recombinant cytokine receptor in vitro and/or in vivo; and/or xii. the level of Treg proliferation increases from re-stimulation in vitro and/or in vivo one or more times.

In some embodiments according to any of the Tregs described above, the Treg cells are cultured in a composition comprising a cytokine other than IL-2.

In some embodiments according to any of the Tregs described above, the population of Treg cells transduced with the recombinant cytokine receptor expands at least about two-fold more than a population of the same Treg cells not transduced with the recombinant cytokine receptor. In some embodiments, the population of Treg cells transduced with the recombinant cytokine receptor expands at least about two-fold, and the population of Treg cells transduced with the recombinant cytokine receptor maintains expression of least one Treg marker selected from the group consisting of CD25, FOXP3, and HELIOS.

### BRIEF DESCRIPTION OF THE DRAWINGS

FIG. 1 shows fluorescence-activated cell sorting (FACS) plots of T regulatory (Treg) cells transduced with an empty vector control (MND Control), IL-2 wild type fused to IL-2Rβ, or an IL-2 mutant fused to IL-2Rβ. The cells were transduced and grown for 14 days, then stained for IL-2Rα and CD28, REH IL-2 mutant is an IL-2 cytokine with the amino acid substitutions L18R, Q22F, and Q126H. 3×IL-2 mutant is an IL-2 cytokine with the amino acid substitutions R38D, K43E, and E61R. 3×REH IL-2 3× is an IL-2 cytokine with the amino acid substitutions L18R, Q22E, Q127H, R38D, K43E, and E61R.

FIG. 2A shows FACS plots of Treg cells transduced with an empty vector control. Cells were transduced and grown for 14 days in the presence of IL-2, then assayed for the presence of FOXP3, HELIOS, CD28, IL-2Rα, and EGFRt. EGFR+ is a marker for a cell transduced with the construct.

FIG. 2B shows FACS plots of Treg cells transduced with constructs expressing 3×-IL-2/IL-2Rβ. Cells were transduced and grown for 14 days in the presence of IL-2, then assayed for the presence of FOXP3, HELIOS, CD28, IL-2Rα, and EGFRt. EGFR+ is a marker for successful transduction.

FIG. 2C shows FACS plots of Treg cells transduced with constructs expressing 3×IL-2/IL-2Rβ. Cells were transduced and grown for 14 days without IL-2, then assayed for the presence of FOXP3, HELIOS, CD28, IL-2Rα, and EGFRt. EGFR+ is a marker for successful transduction.

FIG. 3A shows FACS plots of Treg cells transduced with an empty vector control. Cells were transduced and grown for 14 days in the presence of IL-2, then assayed for the presence of FOXP3, HELIOS, CD28, IL-2Rα, and EGFRt. EGFR+ is a marker for successful transduction.

FIG. 3B shows FACS plots of Treg cells transduced with constructs expressing the 3×REH-IL-2/IL-2Rβ fusion construct. Cells were transduced and grown for 14 days in the presence of IL-2, then assayed for the presence of FOXP3, HELIOS, CD28, IL-2Rα, and EGFRt. REH 3×IL-2 is an IL-2 cytokine with the amino acid substitutions L18R, Q22E, Q127H, R38D, K43E, and E61R.

FIG. 3C shows FACS plots of Treg cells transduced with constructs expressing 3×REH-IL-2/IL-2Rβ fusion construct. Cells were transduced and grown for 14 days without IL-2, then assayed for the presence of FOXP3, HELIOS, CD28, IL-2Rα, and EGFRt. EGFR+ is a marker for successful transduction. REH 3×IL-2 is an IL-2 cytokine with the amino acid substitutions L18R, Q22E, Q127H, R38D, K43E, and E61R.

FIG. 4 shows a chart indicating the percent of cells positive for EGFRt days post transduction when grown with or without exogenous IL-2. Average EGFRt percent was calculated from 5 donors. Percent was determined by FACS staining for EGFRt. EGFR+ is a marker for a cell transduced with the construct.

FIG. 5A shows the fold change of Treg expansion over a period of days post transduction with control or a 3×IL-2/IL-2Rβ construct when grown with or without exogenous IL-2. Fold expansion was normalized for EGFRt expression across four donors. EGFR+ is a marker for successful transduction. FIG. 5B shows a bar graph representing the cell fold expansion in vitro for control Tregs cultured in the presence of IL-2 cytokine, Tregs expressing 3×/REH IL-2/IL-2Rβ recombinant cytokine receptors cultured in the presence of IL-2, and Tregs expressing 3×/REH IL-2/IL-2Rβ recombinant cytokine receptors cultured without IL-2.

FIG. 6 shows FACS plots of Treg cells transduced with an empty vector control or 3×IL-2/IL-2Rβ recombinant cytokine receptor construct. Cells were transduced and grown for 23 days with or without IL-2, then assayed for the presence of FOXP3, HELIOS, CD28, IL-2Rα, and EGFRt. EGFR+ is a marker for successful transduction.

FIG. 7A shows that Tregs expressing various mutant IL-2 fused to IL-2Rβ can suppress the expansion of CD4+ and CD8+ cells when co-cultured at various ratios. Peripheral blood mononuclear cells (PBMCs) are used as a negative control. FIG. 7B shows the suppressive activity of Tregs expressing 3×/REH IL-2/IL-2Rβ recombinant cytokine receptors on effector CD4+ T cells and effector CD8+ T cells.

FIG. 8A shows FACS plots sorting for STAT5 phosphorylation (pSTAT5) in Tregs expressing various 3×IL-2/IL-2Rβ recombinant cytokine receptor constructs. Concentration of the exogenous IL-2 pulse is indicated next to the corresponding curve. EGFR+ is a marker for successful transduction. FIGS. 8B-8C show histogram (FIG. 8B) and bar graph (FIG. 8C) representation of pSTAT5 levels in EGFR-positive control and 3×/REH IL-2/IL-2Rβ recombinant cytokine receptor-expressing Tregs cultured with or without IL-2 in vitro.

FIG. 9A shows FACS plots sorting for pSTAT5 in EGFRt negative cells that were grown Tregs expressing various 3×IL-2/IL-2Rβ recombinant cytokine receptor constructs. Cells were pulsed with various concentrations of exogenous IL-2 prior to analysis. Concentration of the exogenous IL-2 pulse is indicated next to the corresponding curve. EGFR+ is a marker for successful transduction. FIGS. 9B-9C show histogram (FIG. 9B) and bar graph (FIG. 9C) representation of pSTAT5 levels in EGFR-negative control and 3×/REH IL-2/IL-2Rβ recombinant cytokine receptor-expressing Tregs cultured with or without IL-2 in vitro.

FIG. 10A shows % methylation at indicated CpG sites within the FOXP3 locus for various 3×IL-2/IL-2Rβ recombinant cytokine receptor constructs cultured with or without exogenous IL-2 cytokine. 200,000 cells were harvested at 14 days then sent for TSDR analysis. Tregs derived from two donors were analyzed. FIG. 10B provides a bar graph representing the quantification of the percent methylation at the TSDR FOXP3 locus in conventional CD4 T cells, control Tregs, and Tregs expressing 3×/REH IL-2/IL-2Rβ recombinant cytokine receptors.

FIG. 11 shows the concentration of cytokines secreted into the media by Tregs expressing various 3×IL-2/IL-2Rβ constructs. Cells were re-stimulated on Day 16 with 1:5 bead to cell ratio. Supernatant collected on Day 19 and cytokine production measured by ELISA.

FIGS. 12A-12D show the levels of Treg lineage and activation markers on control and 3×/REH IL-2/IL-2Rβ recombinant cytokine receptor-expressing Tregs cultured with PBMCs at PBMC:Treg ratios of 1:1, 1:2, 1:4 or without PBMCs, including FOXP3 (FIG. 12A), CD25 (FIG. 12B). CTLA4 (FIG. 12C), and GARP (FIG. 12D).

FIG. 13A provides a schematic overview of the Treg (re)stimulation experiment testing Treg response to anti-CD3/anti-CD28 antibody versus CD19 antigen stimulation in vitro. FIG. 13B shows the proliferation over time of control and 3×/REH IL-2/IL-2Rβ recombinant cytokine receptor-expressing Tregs cultured with or without IL-2 in vitro. FIG. 13C shows representative FACS plots comparing FOXP3+ and HELIOS+ levels in control versus 3×REH IL-2/IL-2Rβ recombinant cytokine receptor-expressing Tregs after the first round versus the third round of stimulation by either anti-CD3/anti-CD28 antibodies or by CD19-antigen-specific activation. FIG. 13D shows representative FACS plots of FOXP3 and HELIOS levels in control Tregs and Tregs expressing 3×REH IL-2/IL-2Rβ recombinant cytokine receptors in cells grown in vitro under control conditions and under pro-inflammatory conditions at day 3 and day 17. Cells that were grown in the pro-inflammatory condition were treated with 50 ng/mL IL-1β, 50 ng/mL IL-6, 50 ng/mL IL-23, and 300 IU IL-2. Cells were further activated by polyclonal anti-CD3/anti-CD28 antibody stimulation or by CD19-antigen stimulation. FIG. 13E shows a graphical representation of FOXP3 mean fluorescence intensity (MFI) over time for control, IL-12, and pro-inflammatory conditions upon either anti-CD3/anti-CD28 antibody activation or CD19-antigen-specific activation. The IL-12 condition included cell incubation with 20 ng/mL IL-12 and 300 IU IL-2.

FIGS. 14A-14C show the average mean fluorescence intensity (MFI) over time of markers of Treg proliferation, activation, or exhaustion such as CD71 (FIG. 14A), ICOS (FIG. 14B), and PD-1 (FIG. 14C) for control and 3×/REH IL-2/IL-2Rβ recombinant cytokine receptor-expressing Tregs cultured with or without IL-2 in vitro using the stimulation conditions indicated in FIG. 13A.

FIGS. 15A-15C show the percent of Tregs producing cytokines, such as IL-10 (FIG. 15A), IFN-γ (FIG. 15B), and GrB (FIG. 15C) for control and 3×/REH IL-2/IL-2Rβ recombinant cytokine receptor-expressing Tregs cultured with IL-2 in vitro at Days 2 and 19.

FIG. 16A shows the absolute number of Tregs that engrafted and expanded in vivo in an HGD mouse model where mice are immunized with CD19 or vehicle and then intravenously injected with control or 3×/REH IL-2/IL-2Rβ recombinant cytokine receptor-expressing Tregs with or without CD19-CAR expression, as shown in the lower panel schematic. The left panel shows results of cell expansion in the liver at 30 days, and the right panel shows results of cell expansion in the liver at 60 days post-Treg adoptive transfer. FIG. 16B shows the Treg phenotype via FOXP3 and HELIOS levels in control and 3×/REH IL-2/IL-2Rβ recombinant cytokine receptor-expressing Tregs found in the liver, lung, bone marrow, or spleen on Day 30 or Day 60 post-Treg adoptive transfer in vivo. FIG. 16C (graphical representation) and FIG. 16D (FACS plot) show the number of human immune cells and percent of those immune cells that are Treg cells circulating in the blood at 15 days post adoptive cell transfer, with versus without exogenous IL-2.

FIG. 17 shows the in vivo suppressive activity of Tregs expressing 3×/REH IL-2/IL-2Rβ recombinant cytokine receptors in a graft versus host disease (GVHD) humanized murine model. PBMC, peripheral blood mononuclear cells: 5M, 5 million (5×10⁶).

FIG. 18 shows the progression of EAE score over time as an indicator of neurological autoimmunity severity (i.e., multiple sclerosis) in control mice and mice with constitutively active STAT5b (CA-STAT5b) in cells that express FOXP3 (i.e., Tregs).

FIGS. 19A-19B show the fold expansion as bar graph (FIG. 19A) and FACS plot (FIG. 19B) for wildtype (WT) Tregs with or without IL-2 and CA-STAT5b Tregs without IL-2. FIGS. 19C-19D show the suppressive activity of WT Tregs and CA-STAT5b Tregs in vitro (FIG. 19C and in an in vivo graft versus host disease (GVHD) murine model (FIG. 19D).

FIGS. 20A-20B show the persistence and expansion of IL-2 secreting Tregs. FIG. 20A shows the percent of IL-2 secreting Tregs cultured with or without IL-2 that persist over time. FIG. 20B shows the percent of IL-2 secreting Tregs that have expanded when cultured with (bottom panel) or without (top panel) IL-2.

FIG. 21A shows the cell count over time for control Tregs and Tregs expressing IL-2 tethered protein tags cultured with or without IL-2. FIG. 21B shows FACS analysis over time of IL-2 and EGFR protein tag levels in Tregs expressing IL-2 tethered EGFR that were grown without IL-2 in vitro. FIG. 21C provides a bar graph showing the amount of soluble IL-2 accumulating over time in the media of Tregs expressing IL-2 tethered protein tags that were grown without IL-2 in vitro.

FIG. 22 shows a heatmap of RNA sequencing analysis performed on wildtype Tregs and CA-STAT5b Tregs. 278 differentially expressed genes (DEGs) were identified, of which 99 were downregulated and 179 were upregulated in the CA-STAT5b Tregs compared to wildtype Tregs.

FIG. 23 shows a heatmap of RNA sequencing analysis performed on wildtype Tregs and Tregs expressing 3×/REH IL-2/IL-2Rβ recombinant cytokine receptors, both of which were cultured with IL-2. Thirty-four differentially expressed genes (DEGs) were identified, of which 13 were downregulated and 21 were upregulated in the Tregs expressing 3×/REH IL-2/IL-2Rβ recombinant cytokine receptors compared to wildtype Tregs.

### DETAILED DESCRIPTION

Successful adoptive cell therapy requires a robust expansion and persistence of administered cells, and the environmental signals received by the cell contribute heavily to these behaviors. The present invention relates to recombinant cytokine receptors that promote IL-2 signaling in target cells (e.g., in Tregs) in the absence of exogenous IL-2. This IL-2 signaling cascade promotes cell survival, cell expansion, and cell function. In particular, this invention relates to IL-2Rβ protein, or polypeptide, chains tethered to IL-2 at the N-terminus of the IL-2Rβ extracellular domain. In some instances, the tethered IL-2 molecule is mutated to reduce or abolish IL-2 cytokine binding to IL-2Rα and/or IL-2Rγ chains, as described herein. Provided herein in some embodiments are systems in which the proliferation of immune cells (e.g., T cells, such as Tregs) along with their persistence in vivo, production of immune activating cytokines, and immune function can be enhanced by the introduction of recombinant cytokine receptors. These recombinant cells may be used as a cell therapy, for example, autoimmune disorders.

Furthermore, the recombinant cytokine receptors demonstrate different functional properties that are dependent upon the tethered IL-2 cytokine or variant thereof. For example, Treg cells that express the 3×IL-2/IL-2Rβ or 3×REH IL-2/IL-2Rβ recombinant cytokine receptors display the highest degree of Treg activation. Tregs that express the 3×REH IL-2/IL-2Rβ recombinant cytokine receptors show the highest percentage of FOXP3+ and HELIOS+ co-expression, or Treg stability, as well as suppressive activity against CD4+ and CD8+ conventional T cells. The level of STAT-5 activation as measured by the levels of phosphorylated STAT-5 species, are approximately similar levels between each of the described recombinant cytokine receptors provided herein. As described herein, each of the four IL-2 molecules (i.e., wild-type, 3×, REH, or 3×REH) tethered to the recombinant cytokine receptors have unique IL-2 receptor chain binding properties, resulting in unique biological responses upon activating the IL-2 receptor and downstream signaling cascade. Accordingly, the in vitro and in vivo functional features of the recombinant cytokine receptors were surprising, and these differences can be leveraged to meet the biological constraints of different diseases where Treg targeting may prove beneficial for therapeutic purposes.

In some embodiments, the recombinant cytokine receptors lack a TCR activation or TCR co-activation domain to promote cell function in the absence of T cell receptor or CAR stimulation (e.g., lack any CD3 or CD28 activation or co-stimulatory domains). The cells, in particular the Tregs, that comprise these recombinant cytokine receptors, are still activated, e.g., by canonical TCR activation pathways, e.g., in the presence of target antigens (including CAR activation), in order to induce (i.e., turn on or increase) their suppressive activity. Thus, the Tregs are not able to become activated in the absence of target activation, for example target antigen presentation by an antigen presenting cell (APC) or the presence of one or more polypeptides containing the necessary activation domains. In some embodiments, the recombinant cytokine receptors lacking any co-activation or activation domain comprise a tethered wild-type IL-2 molecule.

Various proposed methods of activating the IL-2 signaling pathway within Tregs failed to show functional success. See Example 9, wherein three separate approaches each failed to support Treg function: a constitutively active STAT5 Treg model, an IL-2 secreting Treg model, and an IL-2 tethered tag protein Treg model each failed to promote Treg activity against conventional T cells, thereby demonstrating that not every approach is successful when introduced into Treg cells. Treg cells are known to be quite different in terms of function, molecular profile, and genetic profile compared to conventional T cells (for example, see Grinberg-Bleyer et al. Cell 170(6): 1096-1108, 2017; Grinberg-Bleyer et al. J Immunol 200(7): 2362-71, 2018, both hereby incorporated by reference in their entirety), thereby necessitating a Treg-specific tailored approach, as described herein. Further, the biological processes of cellular differentiation, survival, proliferation, and activation are even further unique when comparing T cells as a genus to NK cells as a genus, which would be readily appreciable by one skilled in the art, thereby confirming the importance of a Treg-specific tailored approach. Of particular note is the IL-2 tethered tag protein Treg model, wherein the IL-2 was tethered to a membrane-bound tag protein. In this model, the IL-2 cytokine would be in close proximity to the extracellular domain of the IL-2 receptor to initiate IL-2 binding and signaling within the cell. That this model was not able to support Treg survival over time nor support Treg function is demonstrative of the surprising and beneficial technical features of the present invention.

The present invention relates generally to the field of immunology and relates in part to compositions, and methods for growing, modifying, and expanding cells, including for example, recombinant cytokine receptors that allow immune cells such as Treg cells to proliferate in the absence of exogenous IL-2. Provided herein are recombinant cytokine receptors that activate IL-2Rβ signaling by tethering the IL-2 cytokine to the extracellular domain of the IL-2Rβ (also referred to as “IL-2RB”) polypeptide chain. These recombinant IL-2 cytokine receptors are not dependent on exogenous cytokines for activation. Thus, cells transduced with the IL-2 recombinant cytokine receptors described herein have certain advantages as compared to non-transduced cells, including exogenous IL-2 independence. In some embodiments, the IL-2 molecules tethered to the IL-2 recombinant cytokine receptors that are expressed by the cells transduced with the IL-2 recombinant cytokine receptors do not activate the native IL-2 receptors of non-transduced cells. In some embodiments of the methods and compositions provided herein, a cell is engineered to include an IL-2 recombinant cytokine receptor as described herein. The IL-2 recombinant cytokine receptors described herein provide stimulatory cytokine signals to the cell and improve the efficacy of cell therapy. In some embodiments, the recombinant cytokines of this present application comprise amino acid substitutions in the cytokine domain. These recombinant cytokine receptors may form a complex with IL-2Rγ and enable constitutive IL-2R signaling in the cell. In some embodiments, the recombinant cytokine receptor does not activate signaling of a different IL-2 receptor and does not cause the cell to secrete more one or more cytokines more than a cell cultured with exogenous IL-2 cytokine.

In some embodiments, the IL-2 recombinant cytokine receptor comprises an IL-2 cytokine. In some embodiments, the IL-2 recombinant cytokine receptor comprises an IL-2 cytokine with one or more amino acid substitutions that results in reduced or no binding affinity for IL-2RA (also “IL-2Rα) and/or IL-2Rγ. In some embodiments, the IL-2 cytokine comprises at least one or more amino acid substitution at positions selected from amino acid positions 18, 22, 126, 38, 43, and 61. In some embodiments, the IL-2 cytokine comprises at least one amino acid substitution selected from the group consisting of L18R, Q22E, Q126H, R38D, K43E, and E61R. In some embodiments, the mutant IL-2 molecule comprises the amino acid substitutions L18R, Q22E, and Q126H; and/or R38D, K43E, and E61R. In some embodiments, the IL-2 cytokine comprises one or more amino acid substitutions selected from the group consisting of F42A, F42K, R38D, R38A, E61R, R38D and E61R. K35D, K43E, and K43D. In some embodiments, the IL-2 receptor comprises an IL-2 cytokine comprising one or more of the amino acid substitutions selected from the group consisting of L18R, Q22E, Q126K, Q126H, Q126M, and Q126R. In some embodiments the IL-2 cytokine comprises the amino acid substitutions L18R, Q22E, and Q126K. In some embodiments the IL-2 cytokine comprises the amino acid substitutions L18R, Q22E, and Q126M. In some embodiments the IL-2 cytokine comprises the amino acid substitutions L18R, Q22E, and Q126R. In some embodiments, the IL-2 cytokine with one or more amino acid substitutions displays reduced binding to IL-2Rα and/or reduced binding to IL-2Rγ. In some embodiments, the recombinant cytokine receptor forms a complex with IL-2Rγ. In some embodiments, the cytokine receptor engages in IL-2 signaling in the absence of exogenous IL-2 cytokine. In some embodiments, the recombinant cytokine receptor does not activate IL-2 signaling on a cell that does not comprise the recombinant cytokine receptor. In some embodiments, the recombinant cytokine receptor does not activate signaling of a different IL-2 receptor. In some embodiments, the cell is a T cell, for example a Treg.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications, and other publications that are herein incorporated by reference, the definition set forth herein prevails over the definition that is incorporated herein by reference.

The section headings used herein are for organizational purposes only and are not to be construed as limiting the subject matter described.

### I. Recombinant Cytokine Receptors

The present invention provides recombinant cytokine receptors that transduce intracellular interleukin-2 (IL-2) signaling in the absence of exogenous IL-2. In some embodiments, recombinant cytokine receptors are engineered molecules comprising (I) an IL-2 cytokine, (II) a polypeptide linker, (III) an extracellular domain, (IV) a transmembrane domain, and (V) an intracellular domain. In some embodiments, the recombinant cytokine receptors do not comprise a TCR activation domain or a costimulatory domain (e.g., a CD3 or a CD28 activation or costimulatory domain, such as a CD28 signaling domain). In some embodiments, the IL-2 cytokine is a naturally occurring IL-2 cytokine and/or a naturally occurring IL-2 receptor polypeptide, e.g., a naturally occurring IL-2Rβ. In some embodiments, the recombinant cytokine receptor comprises a naturally occurring IL-2 cytokine but does not comprise a CD28 signaling domain (e.g., a CD28 activation domain or costimulatory domain). In some embodiments, the IL-2 cytokine comprises one or more amino acid substitutions. In some embodiments, the IL-2 cytokine comprises at least one or more amino acid substitution at positions selected from amino acid positions 18, 22, 126, 38, 43, 61, 29, 15, 16, 19, 20, 22, 23, and 81. In some embodiments, the IL-2 cytokine comprises one or more of the following substitutions L18R, Q22E, Q126H. In some embodiments, the IL-2 cytokine comprises one or more of the following substitutions R38D, K43E, and E61R. In some embodiments, the IL-2 cytokine comprises one or more of the substitutions selected from the group consisting of E15S, H16Q, L19V, D20L, M23Q, and R81D. In some embodiments, the IL-2 cytokine comprises one or more of the substitutions selected from the group consisting of E15S, H16Q, L19V, D20L, Q22K, and M23A. In some embodiments, the IL-2 cytokine comprises L18R, Q22E, and Q126H; and/or R38D, K43E, and E61R; and/or E15S, H16Q, L19V, D20L, M23Q, and R81D; and/or E15S, H16Q, L19V, D20L, Q22K, and M23A. In some embodiments, the IL-2 cytokine comprises L18R, Q22E, Q126H, R38D, K43E, and E61R. In some embodiments, the IL-2 cytokine comprises L18R, Q22E, Q126H, R38D, K43E, E61R, E15S, H16Q, L19V, D20L, M23Q, and R81D. In some embodiments, the IL-2 cytokine comprises L18R, Q22E, Q126H, R38D, K43E, E61R, E15S, H16Q, L19V, D20L, Q22K, and M23A. In some embodiments, the IL-2 cytokine comprises one or more amino acid substitutions selected from the group consisting of F42A, F42K, R38D, R38A, E61R, R38D and E61R, K35D, K43E, K43D, E15S, H16Q, L19V, D20L, Q22K, M23Q, M23A, and R81D. In some embodiments, the IL-2 receptor comprises an IL-2 cytokine comprising one or more of the amino acid substitutions selected from the group consisting of L18R, Q22E, Q126K, Q126H, Q126M, and Q126R. In some embodiments the IL-2 cytokine comprises the amino acid substitutions L18R, Q22E, and Q126K. In some embodiments the IL-2 cytokine comprises the amino acid substitutions L18R, Q22E, and Q126M. In some embodiments the IL-2 cytokine comprises the amino acid substitutions L18R, Q22E, and Q126R. In some embodiments, the IL-2 cytokine comprises an amino acid sequence comprising at least about 80%, at least about 85%, at least about 90%, at least about 95%, at least about 96%, at least about 97%, at least about 98%, at least about 99% or about 100% sequence identity with the amino acid sequence selected from the group consisting of SEQ ID NOs:4-6 or 12. In some embodiments, the IL-2 cytokine is tethered to the IL-2 receptor extracellular domain, e.g., the IL-2Rβ extracellular domain, by a polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the IL-2 receptor extracellular domain comprises the IL-2Rβ extracellular domain. In some embodiments, the IL-2 receptor extracellular domain is the IL-2Rβ extracellular domain. In some embodiments, the IL-2 receptor extracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, provided herein are receptors comprising an IL-2Rβ polypeptide tethered at its N-terminus to an IL-2 cytokine, which is therefore able to be activated in the absence of any exogenous IL-2 cytokine. Recombinant cytokine receptors described herein are derived from Interleukin-2 cytokine receptors, e.g., the IL-2Rβ, wherein the IL-2 tethered receptor is the Interleukin-2 Receptor beta (IL-2Rβ, or IL-2RB) chain of the IL-2R complex or a derivative thereof (e.g., an IL-2Rβ polypeptide comprising at least about 1, 2, 3, 4, 5, 6, 7, 8, 9, or 10 amino acid variations such as substitutions, deletions, or additions compared to a naturally occurring IL-2Rβ polypeptide sequence). In some instances, amino acid variations may be introduced in the IL-2Rβ intracellular domain, for example to optimize, enhance, promote, increase, etc. intracellular IL-2 signaling, such as STAT5 phosphorylation. In other instances, amino acid variations may be introduced in the IL-2Rβ extracellular domain, for example to alter binding affinity to an IL-2 cytokine, for example to reduce binding to a naturally occurring IL-2 cytokine and simultaneously increase binding to a non-naturally occurring IL-2 cytokine, such as any non-naturally occurring (e.g., “mutant” or “mutated”) IL-2 cytokine described herein. In some instances, amino acid variations may be introduced in both the IL-2Rβ intracellular domain and the IL-2Rβ extracellular domain. Canonical IL-2 signaling causes STAT5 phosphorylation, thereby activating STAT5 nuclear translocation and initiation of target gene transcription. In some cells, e.g., T cells, including Treg cells, IL-2 signaling is required for cell survival and cell proliferation. Therefore, recombinant cytokine receptors comprising an IL-2Rβ polypeptide that is tethered to an IL-2 cytokine are able to activate cell survival and proliferation in the absence of exogenous IL-2 via preferentially binding to the tethered IL-2 cytokine.

In some embodiments, IL-2 tethered recombinant cytokine receptors described herein are described with respect to the tethered IL-2 cytokine. Thus, for example, an “WT IL-2-tethered” receptor comprises a wild-type IL-2 molecule linked to the IL-2Rβ polypeptide. In some embodiments, the recombinant cytokine receptor comprises an IL-2 cytokine with one or more amino acid substitutions that results in reduced or no binding affinity for the IL-2Rα (alternatively “IL-2RA”) polypeptide chain with minimal or no disruption of binding to the IL-2Rβ polypeptide chain, for example, reduced binding affinity for IL-2Rα to less than at least about 50%, about 45%, about 40%, about 35%, about 30%, about 25%, about 20%, about 15%, about 10%, about 9%, about 8%, about 7%, about 6%, about 5%, about 4%, about 3%, about 2%, about 1% or less than WT IL-2 binding affinity for IL-2Rα. In some embodiments, the IL-2 cytokine comprises at least one amino acid substitution that reduces affinity for IL-2Rα by at least about any of 1.5-fold. 1.6-fold, 1.7-fold, 1.8-fold, 1.9-fold, 2-fold, 2.5-fold, 3-fold, 3.5-fold, 4-fold, 4.5-fold, S-fold, 6-fold, 7-fold, 8-fold, 9-fold, 10-fold, 15-fold, 20-fold, 25-fold, 30-fold, 35-fold-40-fold, 45-fold, 50-fold, or more compared to WT IL-2 binding affinity for IL-2Rα. In some embodiments, the recombinant cytokine receptor comprises an IL-2 cytokine with one or more amino acid substitutions that results in reduced or no binding affinity for the IL-2Rγ polypeptide chain with minimal or no disruption of binding to the IL-2Rβ polypeptide chain, for example, reduced binding affinity for IL-2Rγ to less than at least about 50%, about 45%, about 40%, about 35%, about 30%, about 25%, about 20%, about 15%, about 10%, about 9%, about 8%, about 7%, about 6%, about 5%, about 4%, about 3%, about 2%, about 1% or less than WT IL-2 binding affinity for IL-2Rγ. In some embodiments, the IL-2 cytokine comprises at least one amino acid substitution that reduces affinity for IL-2Rγ by at least about any of 1.5-fold, 1.6-fold, 1.7-fold, 1.8-fold, 1.9-fold, 2-fold, 2.5-fold, 3-fold, 3.5-fold, 4-fold, 4.5-fold, 5-fold, 6-fold, 7-fold, 8-fold, 9-fold, 10-fold, 15-fold, 20-fold, 25-fold, 30-fold, 35-fold-40-fold, 45-fold, 50-fold, or more compared to WT IL-2 binding affinity for IL-2Rγ. In some embodiments, the recombinant cytokine receptor comprises an IL-2 cytokine with one or more amino acid substitutions that displays reduced or no binding affinity for either or both of the IL-2Rα and/or IL-2Rγ polypeptide chains with minimal or no disruption of binding to the IL-2Rβ polypeptide chain, for example, reduced binding affinity for IL-2Rα to less than at least about 50%, about 45%, about 40%, about 35%, about 30%, about 25%, about 20%, about 15%, about 10%, about 9%, about 8%, about 7%, about 6%, about 5%, about 4%, about 3%, about 2%, about 1% or less than WT IL-2 binding affinity for IL-2Rα and/or reduced binding affinity for IL-2Rγ to less than at least about 50%, about 45%, about 40%, about 35%, about 30%, about 25%, about 20%, about 15%, about 10%, about 9%, about 8%, about 7%, about 6%, about 5%, about 4%, about 3%, about 2%, about 1% or less than WT IL-2 binding affinity for IL-2Rγ. In some embodiments, the IL-2 cytokine comprises at least one amino acid substitution that reduces affinity for IL-2Rα by at least about any of 1.5-fold, 1.6-fold, 1.7-fold, 1.8-fold, 1.9-fold, 2-fold, 2.5-fold, 3-fold, 3.5-fold, 4-fold, 4.5-fold, 5-fold, 6-fold, 7-fold, 8-fold, 9-fold, 10-fold, 15-fold, 20-fold, 25-fold, 30-fold, 35-fold-40-fold, 45-fold. 50-fold, or more and/or that reduces affinity for IL-2Rγ by at least about any of 1.5-fold, 1.6-fold, 1.7-fold, 1.8-fold, 1.9-fold, 2-fold, 2.5-fold, 3-fold, 3.5-fold, 4-fold, 4.5-fold, 5-fold, 6-fold, 7-fold, 8-fold, 9-fold, 10-fold, 15-fold, 20-fold, 25-fold, 30-fold, 35-fold-40-fold, 45-fold, 50-fold, or more compared to WT IL-2 binding affinity for IL-2Rα and/or IL-2Rγ.

In some embodiments, the recombinant cytokine receptor comprises an IL-2 cytokine with one or more amino acid substitutions that results in reduced or no binding affinity for the naturally occurring IL-2Rβ, but with enhanced binding affinity for a non-naturally occurring IL-2Rβ. Thus, in some embodiments, the recombinant cytokine receptor comprises both an IL-2 cytokine with one or more amino acid substitutions and an IL-2Rβ polypeptide with one or more amino acid substitutions. For example, in some embodiments, the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. See, e.g., Zhang et al., Sci Transl Med 13, eabg6986 (2021); and Sockolosky et al., Science 359, 1037-1042 (2018), hereby both incorporated by reference in their entirety. Human IL-2 can be mutated at positions i. E15S, H16Q, L19V, D20L, M23Q, and R81D, or ii. E15S, H16Q, L19V, D20L, Q22K, and M23A, and binds to human IL-2Rβ comprising mutations H133D and Y134F. In some embodiments, the IL-2 cytokine comprises at least one amino acid substitution that reduces affinity for naturally occurring IL-2Rβ to less than at least about 50%, about 45%, about 40%, about 35%, about 30%, about 25%, about 20%, about 15%, about 10%, about 9%, about 8%, about 7%, about 6%, about 5%, about 4%, about 3%, about 2%, about 1% or less than WT IL-2 binding affinity for naturally occurring IL-2Rβ. In some embodiments, the IL-2 cytokine comprises at least one amino acid substitution that reduces affinity for naturally occurring IL-2Rβ by at least about any of 1.5-fold, 1.6-fold. 1.7-fold, 1.8-fold, 1.9-fold, 2-fold, 2.5-fold, 3-fold, 3.5-fold, 4-fold, 4.5-fold, 5-fold, 6-fold, 7-fold, 8-fold, 9-fold, 10-fold, 15-fold, 20-fold, 25-fold, 30-fold, 35-fold-40-fold, 45-fold, 50-fold, or more compared to WT IL-2 binding affinity for naturally occurring IL-2Rβ. In some embodiments, the IL-2Rβ comprises at least one amino acid substitution that reduces affinity for naturally occurring IL-2 cytokine to less than at least about 50%, about 45%, about 40%, about 35%, about 30%, about 25%, about 20%, about 15%, about 10%, about 9%, about 8%, about 7%, about 6%, about 5%, about 4%, about 3%, about 2%, about 1% or less than WT IL-2Rβ binding affinity for naturally occurring IL-2 cytokine. In some embodiments, the IL-2Rβ comprises at least one amino acid substitution that reduces affinity for naturally occurring IL-2 cytokine by at least about any of 1.5-fold, 1.6-fold, 1.7-fold, 1.8-fold, 1.9-fold, 2-fold, 2.5-fold, 3-fold, 3.5-fold, 4-fold, 4.5-fold, 5-fold, 6-fold, 7-fold, 8-fold. 9-fold, 10-fold, 15-fold. 20-fold, 25-fold, 30-fold, 35-fold-40-fold, 45-fold, 50-fold, or more compared to WT IL-2Rβ binding affinity for naturally occurring IL-2 cytokine.

In some embodiments, the recombinant cytokine receptors are more effective for maintaining immune cell persistence. In some embodiments, the recombinant cytokine receptor allows the transduced Treg cell to proliferate without exogenous IL-2. In some embodiments, the recombinant cytokine receptor increases the relative amount of Treg cells in a composition comprising a population of Treg cells transduced with the recombinant cytokine receptor over time. In some embodiments, the immune cell is a T cell. In some embodiments, the immune cell is a Treg.

In some embodiments, the recombinant cytokine receptors are more effective for maintaining Treg persistence compared to other strategies for producing exogenous IL-2 independent Tregs, such as, for example, Tregs that secrete IL-2 or Tregs comprising membrane-bound IL-2 tethered tag proteins. In some embodiments, cells transduced with the recombinant cytokine receptors provided herein are able to maintain a Treg phenotype and/or have increased persistence compared to Treg cells that secrete IL-2 or compared to Tregs comprising membrane-bound IL-2 tethered tag proteins.

In some embodiments, the recombinant cytokine receptors provided herein have significant advantages including, but not limited to: (1) expression in regulatory T cells (Tregs); (2) increasing Treg survival and proliferation in the absence of IL-2; (3) increased IL-2 receptor signaling via STAT5 phosphorylation; (4) ability to suppress effector T cells in the absence of IL-2 to about or approximately the same degree as wild-type Tregs grown in the presence of IL-2; (5) supports Treg expansion and survival for at least about 14 days, and/or at least about 23 days without exogenous IL-2; and (6) supports Treg identity for at least about 14 days, and/or at least about 23 days without exogenous IL-2.

TABLE 1
Exemplary IL-2 tethered recombinant cytokine receptor sequences.

| | | | | IL-2 recombinant |
|---|---|---|---|---|
| | | Cytokine | Linker | cytokine Receptor: |
| Sequence Name | Full Sequence | Sequence | Sequence | IL-2Rβ Sequence |
| “IL-2”; IL-2 | SEQ ID NO: 1 | SEQ ID NO: 4 | SEQ ID NO: 9 | SEQ ID NO: 7 |
| tethered IL-2Rβ | | | | |
| “3x”; 3x IL-2 | SEQ ID NO: 2 | SEQ ID NO: 5 | SEQ ID NO: 9 | SEQ ID NO: 7 |
| tethered IL-2Rβ | | | | |
| “REH”; REH IL-2 | SEQ ID NO: 3 | SEQ ID NO: 6 | SEQ ID NO: 9 | SEQ ID NO: 7 |
| tethered IL-2Rβ | | | | |
| “3x REH”; 3x REH | SEQ ID NO: 11 | SEQ ID NO: 12 | SEQ ID NO: 9 | SEQ ID NO: 7 |
| IL-2 tethered IL-2Rβ | | | | |

Provided herein are recombinant cytokine receptors (e.g., recombinant IL-2 receptors) comprising an IL-2Rβ polypeptide tethered at its N-terminus to an IL-2 cytokine, which is therefore able to be activated in the absence of any exogenous cytokine, e.g., exogenous IL-2. In some embodiments, the recombinant cytokine receptor is able to signal in the absence of exogenous IL-2. In some embodiments, the recombinant cytokine receptor comprises an IL-2 cytokine, a linker polypeptide, an IL-2Rβ extracellular domain, an IL-2Rβ transmembrane domain, and an IL-2Rβ intracellular domain. In some embodiments, the recombinant cytokine receptors do not comprise a TCR activation domain or a costimulatory domain (e.g., a CD3 or a CD28 activation or costimulatory domain, such as a CD28 signaling domain). In some embodiments, the IL-2 receptor binds to an IL-2 cytokine that is linked to the IL-2Rβ polypeptide by a polypeptide linker sequence. Thus, in some embodiments, there is provided a recombinant cytokine receptor comprising from N to C terminus: (I) an IL-2 cytokine, (II) a polypeptide linker, (III) an IL-2 receptor extracellular domain, (IV) an IL-2 receptor transmembrane domain, and (V) an IL-2 receptor intracellular domain. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, there is provided a recombinant cytokine receptor comprising from N to C terminus: (I) an IL-2 cytokine, (II) a polypeptide linker, (III) an IL-2Rβ extracellular domain, (IV) a transmembrane domain, and (V) an IL-2Rβ intracellular domain. In some embodiments, there is provided a recombinant cytokine receptor comprising from N to C terminus: (I) an IL-2 cytokine, (II) a polypeptide linker, and (III) an IL-2Rβ polypeptide. In some embodiments, there is provided a recombinant cytokine receptor comprising from N to C terminus: (I) an IL-2 cytokine comprising an amino acid sequence selected from the group consisting of SEQ ID NOs:4-6 and 12, (II) a polypeptide linker, (III) an IL-2Rβ extracellular domain, (IV) a transmembrane domain, and (V) an IL-2Rβ intracellular domain. In some embodiments, there is provided a recombinant cytokine receptor comprising from N to C terminus: (I) an IL-2 cytokine comprising an amino acid sequence selected from the group consisting of SEQ ID NOs:4-6 and 12, (II) a polypeptide linker, and (III) an IL-2Rβ polypeptide. In some embodiments, the recombinant cytokine receptor comprises the amino acid sequence selected from the group consisting of SEQ ID NOs:1-3 and 11. In some embodiments, the recombinant cytokine receptor comprises an amino acid sequence comprising at least about 80%, at least about 85%, at least about 90%, at least about 95%, at least about 96%, at least about 97%, at least about 98%, at least about 99% or about 100% sequence identity with the amino acid sequence selected from the group consisting of SEQ ID NOs:1-3 and 11. In some embodiments, the recombinant cytokine receptor comprises an IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence selected from the group consisting of SEQ ID NOs:4-6 and 12. In some embodiments, the IL-2 cytokine is tethered to the IL-2 cytokine receptor extracellular domain by the polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the extracellular domain is the IL-2Rβ extracellular domain. In some embodiments, the extracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain comprises the IL-2Rβ transmembrane domain. In some embodiments, the transmembrane domain is the IL-2Rβ extracellular domain. In some embodiments, the transmembrane domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain comprises the IL-2Rβ intracellular domain. In some embodiments, the intracellular domain is the IL-2Rβ intracellular domain. In some embodiments, the intracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO: 15. In some embodiments, there is provided a recombinant cytokine receptor comprising from N to C terminus: (I) an IL-2 cytokine comprising an amino acid sequence selected from the group consisting of SEQ ID NOs:4-6 and 12, (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15. In some embodiments, there is provided a recombinant cytokine receptor comprising from N to C terminus: (I) an IL-2 cytokine comprising an amino acid sequence selected from the group consisting of SEQ ID NOs:4-6 and 12, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7.

In some embodiments, the recombinant IL-2 receptor (i.e., a “recombinant cytokine receptor”) binds to a wild-type IL-2 cytokine that is linked to the IL-2Rβ polypeptide by a polypeptide linker sequence. Thus, in some embodiments, there is provided a recombinant cytokine receptor (i.e., a “recombinant IL-2 receptor”) comprising from N to C terminus: (I) a wild-type IL-2 cytokine; (II) a polypeptide linker, (III) an IL-2Rβ extracellular domain, (IV) a transmembrane domain, and (V) an IL-2Rβ intracellular domain. In some embodiments, the recombinant cytokine receptor does not comprise a T cell receptor (TCR) activation domain or a costimulatory domain (e.g., a CD3 or a CD28 activation or costimulatory domain, such as a CD28 signaling domain). In some embodiments, there is provided a recombinant cytokine receptor comprising from N to C terminus: (I) an IL-2 cytokine comprising the amino acid sequence of SEQ ID NO:4, (II) a polypeptide linker, (III) an IL-2Rβ extracellular domain, (IV) a transmembrane domain, and (V) an IL-2Rβ intracellular domain. In some embodiments, there is provided a recombinant cytokine receptor comprising from N to C terminus: (I) an IL-2 cytokine comprising the amino acid sequence of SEQ ID NO:4, (II) a polypeptide linker, (III) an IL-2Rβ extracellular domain, (IV) a transmembrane domain, and (V) an IL-2Rβ intracellular domain, wherein the recombinant cytokine receptor does not comprise a T cell receptor activation domain or a T cell costimulatory domain, optionally wherein the T cell receptor activation domain or T cell costimulatory domain is a CD28 signaling domain. In some embodiments, there is provided a recombinant cytokine receptor comprising from N to C terminus: (I) an IL-2 cytokine comprising the amino acid sequence of SEQ ID NO:4, (II) a polypeptide linker, and (III) an IL-2Rβ polypeptide. In some embodiments, there is provided a recombinant cytokine receptor comprising from N to C terminus: (I) an IL-2 cytokine comprising the amino acid sequence of SEQ ID NO:4, (II) a polypeptide linker, and (III) an IL-2Rβ polypeptide, wherein the recombinant cytokine receptor does not comprise a T cell receptor activation domain or a T cell costimulatory domain, optionally wherein the T cell receptor activation domain or T cell costimulatory domain is a CD28 signaling domain. In some embodiments, the recombinant cytokine receptor comprises the amino acid sequence set forth in SEQ ID NO:1. In some embodiments, the recombinant cytokine receptor comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:1. In some embodiments, the recombinant cytokine receptor comprises a wild-type IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:4. In some embodiments, the IL-2 cytokine is tethered to the IL-2 cytokine receptor extracellular domain by the polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the extracellular domain is the IL-2Rβ extracellular domain. In some embodiments, the extracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain comprises the IL-2Rβ transmembrane domain. In some embodiments, the transmembrane domain is the IL-2Rβ extracellular domain. In some embodiments, the transmembrane domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain comprises the IL-2Rβ intracellular domain. In some embodiments, the intracellular domain is the IL-2Rβ intracellular domain. In some embodiments, the intracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, there is provided a recombinant cytokine receptor comprising from N to C terminus: (I) an IL-2 cytokine comprising the amino acid sequence of SEQ ID NO:4, (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15. In some embodiments, there is provided a recombinant cytokine receptor comprising from N to C terminus: (I) an IL-2 cytokine comprising the amino acid sequence of SEQ ID NO:4, (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15, wherein the recombinant cytokine receptor does not comprise a T cell receptor activation domain or a T cell costimulatory domain, optionally wherein the T cell receptor activation domain or T cell costimulatory domain is a CD28 signaling domain. In some embodiments, there is provided a recombinant cytokine receptor comprising from N to C terminus: (I) an IL-2 cytokine comprising the amino acid sequence of SEQ ID NO:4, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7. In some embodiments, there is provided a recombinant cytokine receptor comprising from N to C terminus: (I) an IL-2 cytokine comprising the amino acid sequence of SEQ ID NO:4, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7, wherein the recombinant cytokine receptor does not comprise a T cell receptor activation domain or a T cell costimulatory domain, optionally wherein the T cell receptor activation domain or T cell costimulatory domain is a CD28 signaling domain.

In some embodiments, the recombinant IL-2 receptor (i.e., a “recombinant cytokine receptor”) binds to an IL-2 cytokine comprising one or more amino acid substitutions. In some embodiments, the one or more substitutions decreases binding or affinity between i) the IL-2Rα cytokine receptor extracellular domain and the IL-2 cytokine and/or ii) the IL-2Rγ cytokine receptor extracellular domain and the IL-2 cytokine. In some embodiments, the one or more substitutions does not or only minimally decreases binding or affinity between the IL-2Rβ extracellular domain and the IL-2 cytokine. In some embodiments, the IL-2 cytokine comprises at least one or more amino acid substitution at positions selected from amino acid positions 42, 38, 61, 35, 18, 22, 126, and 43. In some embodiments, the IL-2 cytokine comprises at least one or more amino acid substitution at positions selected from amino acid positions 18, 22, 126, 38, 43 and 61. In some embodiments, the IL-2 cytokine is linked to the IL-2Rβ polypeptide by a polypeptide linker sequence, wherein the amino acid substituted IL-2 cytokine comprises one or more of the amino acid substitutions selected from the group consisting of R38D, K43E, and E61R. In some embodiments, the amino acid substituted IL-2 cytokine may comprise additional amino acid substitutions. In some embodiments, the recombinant IL-2 receptor binds to an amino acid substituted IL-2 cytokine that is linked to the IL-2Rβ polypeptide by a polypeptide linker sequence, wherein the mutated IL-2 cytokine comprises the amino acid substitutions R38D, K43E, and E61R. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. Thus, in some embodiments, there is provided a recombinant cytokine receptor (i.e., a “recombinant IL-2 receptor”) comprising from N to C terminus (I) a mutated IL-2 cytokine comprising the amino acid substitutions R38D, K43E, and E61R (“3×IL-2”), (II) a polypeptide linker, (III) an IL-2 receptor extracellular domain, (IV) a transmembrane domain, and (V) an IL-2 receptor intracellular domain. In some embodiments, there is provided a recombinant cytokine receptor comprising from N to C terminus: (I) an IL-2 cytokine comprising the amino acid sequence of SEQ ID NO:5, (II) a polypeptide linker, (II) an IL-2Rβ extracellular domain, (IV) a transmembrane domain, and (V) an IL-2Rβ intracellular domain. In some embodiments, there is provided a recombinant cytokine receptor comprising from N to C terminus: (I) an IL-2 cytokine comprising the amino acid sequence of SEQ ID NO:5, (II) a polypeptide linker, and (III) an IL-2Rβ polypeptide. In some embodiments, the recombinant cytokine receptor comprises the amino acid sequence set forth in SEQ ID NO:2. In some embodiments, the recombinant cytokine receptor comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:2. In some embodiments, the recombinant cytokine receptor comprises a 3×-IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:5. In some embodiments, the IL-2 cytokine is tethered to the IL-2 cytokine extracellular domain by the polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the extracellular domain is the IL-2Rβ extracellular domain. In some embodiments, the extracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain comprises the IL-2Rβ transmembrane domain. In some embodiments, the transmembrane domain comprises the IL-2Rβ transmembrane domain. In some embodiments, the transmembrane domain is the IL-2Rβ extracellular domain. In some embodiments, the transmembrane domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain comprises the IL-2Rβ intracellular domain. In some embodiments, the intracellular domain comprises the IL-2Rβ intracellular domain. In some embodiments, the intracellular domain is the IL-2Rβ intracellular domain. In some embodiments, the intracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, the 3×IL-2 cytokine displays reduced or no binding to IL-2Rα, but does not display reduced or eliminated binding to IL-2Rβ. In some embodiments, there is provided a recombinant cytokine receptor comprising from N to C terminus: (I) an IL-2 cytokine comprising the amino acid sequence of SEQ ID NO:5, (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15. In some embodiments, there is provided a recombinant cytokine receptor comprising from N to C terminus: (I) an IL-2 cytokine comprising the amino acid sequence of SEQ ID NO:5, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7.

In some embodiments, the IL-2 cytokine comprises one or more amino acid substitutions selected from the group consisting of F42A, F42K, R38D, R38A, E61R, R38D and E61R, K35D, K43E, and K43D. In some embodiments, the IL-2 cytokine is tethered to the IL-2 cytokine receptor extracellular domain by the polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the transmembrane domain comprises the IL-2Rβ transmembrane domain. In some embodiments, the intracellular domain comprises the IL-2Rβ intracellular domain. In some embodiments, the cytokine displays reduced or no binding to IL-2Rα but does not display reduced or eliminated binding to IL-2Rβ. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the TL-2Rβ polypeptide comprises one or both amino acid substitutions H133D and/or Y134F.

In some embodiments, the recombinant IL-2 receptor (i.e., a “recombinant cytokine receptor”) comprises an IL-2 cytokine comprising one or more of the amino acid substitutions selected from the group consisting of L18R, Q22E, and Q126H. In some embodiments, the one or more substitutions decreases binding or affinity between i) the IL-2Rα cytokine receptor extracellular domain and the IL-2 cytokine and/or ii) the IL-2Rγ cytokine receptor extracellular domain and the IL-2 cytokine. In some embodiments, the one or more substitutions does not or only minimally decreases binding or affinity between the IL-2Rβ extracellular domain and the IL-2 cytokine. In some embodiments, the mutated IL-2 cytokine may comprise additional amino acid substitutions. In some embodiments, the recombinant IL-2 receptor binds to a mutated IL-2 cytokine that is linked to the IL-2Rβ polypeptide by a polypeptide linker sequence, wherein the mutated IL-2 cytokine comprises the amino acid substitutions L18R, Q22E, and Q126H. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V. D20L, Q22K, M23Q, M23A, and R81D, and the IL-2Rβ polypeptide comprises one or both amino acid substitutions H133D and/or Y134F. Thus, in some embodiments, there is provided a recombinant cytokine receptor (i.e., a “recombinant IL-2 receptor”) comprising from N to C terminus: (I) a mutated IL-2 cytokine comprising the amino acid substitutions L18R, Q22E, and Q126H (“REH IL-2”), (II) a polypeptide linker, (III) an IL-2 receptor extracellular domain, (IV) a transmembrane domain, and (V) an IL-2 receptor intracellular domain. In some embodiments, there is provided a recombinant cytokine receptor comprising from N to C terminus: (I) an IL-2 cytokine comprising the amino acid sequence of SEQ ID NO:6, (II) a polypeptide linker, (III) an IL-2Rβ extracellular domain, (IV) a transmembrane domain, and (V) an IL-2Rβ intracellular domain. In some embodiments, there is provided a recombinant cytokine receptor comprising from N to C terminus: (I) an IL-2 cytokine comprising the amino acid sequence of SEQ ID NO:6, (II) a polypeptide linker, and (III) an IL-2Rβ polypeptide. In some embodiments, the recombinant cytokine receptor comprises the amino acid sequence set forth in SEQ ID NO:3. In some embodiments, the recombinant cytokine receptor comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:3. In some embodiments, the recombinant cytokine receptor comprises an REH IL-2 molecule having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:6. In some embodiments, the IL-2 cytokine is tethered to the IL-2 cytokine extracellular domain by the polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the extracellular domain is the IL-2Rβ extracellular domain. In some embodiments, the extracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain comprises the IL-2Rβ transmembrane domain. In some embodiments, the transmembrane domain comprises the IL-2Rβ transmembrane domain. In some embodiments, the transmembrane domain is the IL-2Rβ extracellular domain. In some embodiments, the transmembrane domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain comprises the IL-2Rβ intracellular domain. In some embodiments, the intracellular domain comprises the IL-2Rβ intracellular domain. In some embodiments, the intracellular domain is the IL-2Rβ intracellular domain. In some embodiments, the intracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, the REH IL-2 cytokine displays reduced or no binding to IL-2Rα and/or IL-2Rγ, but does not display reduced or eliminated binding to IL-2Rβ. In some embodiments, there is provided a recombinant cytokine receptor comprising from N to C terminus: (I) an IL-2 cytokine comprising the amino acid sequence of SEQ ID NO:6, (I) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15. In some embodiments, there is provided a recombinant cytokine receptor comprising from N to C terminus: (I) an IL-2 cytokine comprising the amino acid sequence of SEQ ID NO:6, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7.

In some embodiments, the recombinant IL-2 receptor comprises an IL-2 cytokine comprising one or more of the amino acid substitutions selected from the group consisting of L18R, Q22E, Q126K, Q126H, Q126M, and Q126R. In some embodiments the IL-2 cytokine comprises the amino acid substitutions L18R, Q22E, and Q126K. In some embodiments the IL-2 cytokine comprises the amino acid substitutions L18R, Q22E, and Q126M. In some embodiments the IL-2 cytokine comprises the amino acid substitutions L18R, Q22E, and Q126R. In some embodiments, the IL-2 cytokine is tethered to the IL-2 cytokine receptor extracellular domain by the polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the transmembrane domain comprises the IL-2Rβ transmembrane domain. In some embodiments, the intracellular domain comprises the IL-2Rβ intracellular domain. In some embodiments, the IL-2 cytokine displays reduced or no binding to IL-2Rα and/or IL-2Rγ but does not display reduced or eliminated binding to IL-2Rβ. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R8I D, and the IL-2Rβ polypeptide comprises one or both amino acid substitutions H133D and/or Y134F.

In some embodiments, the recombinant IL-2 receptor (i.e., a “recombinant cytokine receptor”) comprises an IL-2 cytokine comprising one or more of the amino acid substitutions selected from the group consisting of L18R, Q22E, Q126H, R38D, K43E, and E61R. In some embodiments, the IL-2 cytokine may comprise additional amino acid substitutions. In some embodiments, the recombinant IL-2 receptor binds to a mutated IL-2 cytokine that is linked to the IL-2Rβ polypeptide by a polypeptide linker sequence, wherein the mutated IL-2 cytokine comprises the amino acid substitutions L18R, Q22E, Q126H, R38D, K43E, and E61R. In some embodiments, the one or more substitutions decreases binding or affinity between i) the IL-2Rα cytokine receptor extracellular domain and the IL-2 cytokine and/or ii) the IL-2Rγ cytokine receptor extracellular domain and the IL-2 cytokine. In some embodiments, the one or more substitutions does not or only minimally decreases binding or affinity between the IL-2Rβ extracellular domain and the IL-2 cytokine. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. Thus, in some embodiments, there is provided a recombinant cytokine receptor (i.e., an “recombinant IL-2 receptor”) comprising from N to C terminus: (I) a mutated IL-2 cytokine comprising the amino acid substitutions L18R, Q22E, Q126H, R38D, K43E, and E61R (“3×REH IL-2”), (II) a polypeptide linker, (IIII) an IL-2 receptor extracellular domain, (IV) a transmembrane domain, and (V) an IL-2 receptor intracellular domain. In some embodiments, there is provided a recombinant cytokine receptor comprising from N to C terminus: (I) an IL-2 cytokine comprising the amino acid sequence of SEQ ID NO:12, (II) a polypeptide linker, (III) an IL-2Rβ extracellular domain, (IV) a transmembrane domain, and (V) an IL-2Rβ intracellular domain. In some embodiments, there is provided a recombinant cytokine receptor comprising from N to C terminus: (I) an IL-2 cytokine comprising the amino acid sequence of SEQ ID NO:12, (II) a polypeptide linker, and (III) an IL-2Rβ polypeptide. In some embodiments, the recombinant cytokine receptor comprises the amino acid sequence set forth in SEQ ID NO:11. In some embodiments, the recombinant cytokine receptor comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:11. In some embodiments, the recombinant cytokine receptor comprises a 3×REH IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:12. In some embodiments, the IL-2 cytokine is tethered to the IL-2 cytokine receptor extracellular domain by the polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the extracellular domain is the IL-2Rβ extracellular domain. In some embodiments, the extracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain comprises the IL-2Rβ transmembrane domain. In some embodiments, the transmembrane domain comprises the IL-2Rβ transmembrane domain. In some embodiments, the transmembrane domain is the IL-2Rβ extracellular domain. In some embodiments, the transmembrane domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain comprises the IL-2Rβ intracellular domain. In some embodiments, the intracellular domain comprises the IL-2Rβ intracellular domain. In some embodiments, the intracellular domain is the IL-2Rβ intracellular domain. In some embodiments, the intracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, the 3×REH IL-2 cytokine displays reduced or no binding to IL-2Rα and/or IL-2Rγ, but does not display reduced or eliminated binding to IL-2Rβ. In some embodiments, there is provided a recombinant cytokine receptor comprising from N to C terminus: (I) an IL-2 cytokine comprising the amino acid sequence of SEQ ID NO:12, (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15. In some embodiments, there is provided a recombinant cytokine receptor comprising from N to C terminus: (I) an IL-2 cytokine comprising the amino acid sequence of SEQ ID NO:12, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7.

In some embodiments, cells such as T cells, for example Treg cells, comprise the recombinant cytokine receptor(s) described herein. Thus, in some embodiments, there is provided a T cell (e.g., a Treg cell) comprising a recombinant cytokine receptor comprising from N to C terminus: (I) a WT IL-2 cytokine, a 3×IL-2 cytokine, an REH IL-2 cytokine, or a 3×REH IL-2 cytokine; (II) a polypeptide linker, (III) an IL-2 receptor extracellular domain; (IV) a transmembrane domain; and (V) an IL-2 receptor intracellular domain. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, there is provided a T cell (e.g., a Treg cell) comprising a recombinant cytokine receptor comprising from N to C terminus: (I) an IL-2 cytokine comprising an amino acid sequence selected from the group consisting of SEQ ID NOs:4-6 and 12, (II) a polypeptide linker, (III) an IL-2Rβ extracellular domain, (IV) a transmembrane domain, and (V) an IL-2Rβ intracellular domain. In some embodiments, there is provided a T cell (e.g., a Treg cell) comprising a recombinant cytokine receptor comprising from N to C terminus: (I) an IL-2 cytokine comprising an amino acid sequence selected from the group consisting of SEQ ID NOs:4-6 and 12. (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15. In some embodiments, there is provided a T cell (e.g., a Treg cell) comprising a recombinant cytokine receptor comprising an amino acid sequence selected from the group consisting of SEQ ID NOs:1-3 and 11. In some embodiments, the recombinant cytokine receptor does not comprise a CD28 signaling domain.

In some embodiments, the cells expressing the recombinant cytokine receptor survive and proliferate in the absence of exogenous IL-2. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the cells transduced with the recombinant cytokine receptor remain viable in the absence of exogenous IL-2. In some embodiments, the cells are T cells. In some embodiments, the cells are Treg cells.

In some embodiments, the Tregs expressing the recombinant cytokine receptor survive and proliferate in the absence of exogenous IL-2. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor remain viable in vitro for at least about 2 days to about 23 days after transduction. In some embodiments, at least about 60%-99% of the Tregs transduced with the recombinant cytokine receptor remain viable in vitro for at least about 14 to about 23 days after transduction. In some embodiments, at least about 60-90% of the Tregs transduced with the recombinant cytokine receptor remain viable in vitro for at least about 14 to about 23 days after transduction. In some embodiments, at least about 60-80% of the Tregs transduced with the recombinant cytokine receptor remain viable in vitro for at least about 14 to about 23 days after transduction. In some embodiments, at least about 60%-99% of the Tregs transduced with the recombinant cytokine receptor remain viable in vitro for at least about 14 days after transduction. In some embodiments, at least about 60-90% of the Tregs transduced with the recombinant cytokine receptor remain viable in vitro for at least about 14 after days after transduction. In some embodiments, at least about 60-80% of the Tregs transduced with the recombinant cytokine receptor remain viable in vitro for at least about 14 days after transduction. In some embodiments, the viability of Tregs transduced with the recombinant cytokine receptor is increased compared to Treg cells that are not transduced with the recombinant cytokine receptor. In some embodiments, the viability of Tregs transduced with the recombinant cytokine receptor is increased compared to Treg cells that are not transduced with the recombinant cytokine receptor when cultured without the cognate receptor cytokine, i.e., the IL-2 cytokine. In some embodiments, the viability of Tregs is increased at least about 1.1-fold, at least about 1.5-fold, at least about 2-fold, at least about 3-fold, at least about 3.2-fold, at least about 4-fold, at least about 10-fold, at least about 20-fold, at least about 25-fold, at least about 30-fold, at least about 35-fold, at least about 45-fold, at least about 50-fold, or more compared to untransduced T cells (such as Tregs).

In some embodiments, the Tregs expressing the recombinant cytokine receptor survive and proliferate in the absence of exogenous IL-2. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor persist in vivo for at least about 3 days or longer. In some embodiments, at least about 60%-99% of the Tregs transduced with the recombinant cytokine receptor persist in vivo for at least about 3 days or longer. In some embodiments, at least about 60-90% of the Tregs transduced with the recombinant cytokine receptor persist in vivo for at least about 3 days or longer. In some embodiments, at least about 60-80% of the Tregs transduced with the recombinant cytokine receptor persist in vivo for at least about 3 days or longer.

In some embodiments, the cells transduced with the recombinant cytokine receptor have increased or sustained proliferation post-transduction and in vivo. In some embodiments, the cells transduced with the recombinant cytokine receptor have more cells in an in vitro culture compared to the number of cells before transduction. In some embodiments, the cells are T cells. In some embodiments, the cells are Treg cells.

In some embodiments, the Tregs transduced with the recombinant cytokine receptor have increased or sustained proliferation post-transduction. In some embodiments, the Tregs transduced with the recombinant cytokine receptor have more cells compared to the number of cells before transduction. In some embodiments, the Tregs transduced with recombinant cytokine receptor have about as many cells compared to the number of cells of a Treg not expressing the recombinant cytokine receptor grown with IL-2 over time. In some embodiments, the time period is about 5-14 days. In some embodiments, the time period is about 9-14 days. In some embodiments, Tregs transduced with the recombinant cytokine receptor have a higher rate of proliferation compared to untransduced Treg cells. In some embodiments, the number of cells post-transduction is increased at least about 1.1-fold. at least about 1.5-fold, at least about 2-fold, at least about 5-fold, at least about 10-fold, at least about 20-fold, at least about 50-fold, at least about 75-fold, or more compared to untransduced cells. In some embodiments, the cells are cultured post-transduction without cytokine.

In some embodiments, cell viability, transduction of intracellular signaling, activation of intracellular signaling, or the ability to proliferate is increased upon transduction with the recombinant cytokine receptor.

In some embodiments, cell viability, transduction of intracellular signaling, activation of intracellular signaling, or the ability to proliferate is increased upon transduction with the recombinant cytokine receptor. In some embodiments, the recombinant cytokine receptor expressed in cells transduces intracellular IL-2 signaling within the cells. In some embodiments, the recombinant cytokine receptor activates intracellular IL-2 signaling in cells. In some embodiments, the recombinant cytokine receptor promotes intracellular IL-2 signaling in cells. In some embodiments, the recombinant cytokine receptor increases intracellular IL-2 signaling in cells compared to cells without the recombinant cytokine receptor. In some embodiments, the recombinant cytokine receptors provided herein comprise an active IL-2Rβ polypeptide. In some embodiments, the recombinant cytokine receptors comprise an active IL-2Rβ polypeptide tethered to a wild-type IL-2 molecule (hereinafter “WT IL-2”). In some embodiments, the recombinant cytokine receptors comprise an active IL-2Rβ polypeptide tethered to a mutated IL-2 cytokine comprising one or more amino acid substitutions selected from the group consisting of L18R, Q22E, Q126H, R38D, K43E, and E61R. In some embodiments, the recombinant cytokine receptors comprise an active IL-2Rβ polypeptide tethered to a mutated IL-2 cytokine comprising amino acid substitutions R38D, K43E, and E61R (hereinafter “3×IL-2”). In some embodiments, the recombinant cytokine receptors comprise an active IL-2Rβ polypeptide tethered to a mutated IL-2 cytokine comprising amino acid substitutions L18R, Q22E, and Q126H (hereinafter “REH IL-2”). In some embodiments, the recombinant cytokine receptors comprise an active IL-2Rβ polypeptide tethered to a mutated IL-2 cytokine comprising amino acid substitutions L18R, Q22E, Q1261, R38D, K43E, and E61R (hereinafter “3×REH IL-2”). In some embodiments, the mutated IL-2 cytokine may comprise additional amino acid substitutions. For example, in some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the recombinant cytokine receptor does not comprise a T cell receptor activation domain or a T cell costimulatory domain, optionally wherein the T cell receptor activation domain or T cell costimulatory domain is a CD28 signaling domain. In some embodiments, the tethered recombinant cytokine receptors provided herein are able to engage in downstream signal transduction. In some embodiments, the recombinant cytokine receptor comprises an IL-2Rβ polypeptide which phosphorylates STAT5 through its activation of the JAK1 kinase upon recombinant cytokine receptor activation. In some embodiments, the IL-2Rβ polypeptide phosphorylates STAT5 through its activation of the JAK1 kinase upon recombinant cytokine receptor activation. In some embodiments, the recombinant cytokine receptor phosphorylates Shc upon recombinant cytokine receptor activation, thereby activating the downstream PI3K-AKT pathway. In some embodiments, the recombinant cytokine receptor phosphorylates She upon recombinant cytokine receptor activation, thereby activating the downstream Ras/MAPK pathway. In some embodiments, the recombinant cytokine receptor comprises an IL-2 cytokine. In some embodiments, the IL-2 cytokine is a mutated IL-2 molecule. In some embodiments, the mutated IL-2 molecule with one or more amino acid substitutions displays reduced or no binding to IL-2Rα and/or polypeptides. In some embodiments, the mutated IL-2 cytokine known as “3×IL-2” displays reduced or no binding to IL-2Rα. In some embodiments, the mutated IL-2 cytokines known as “REH IL-2” and “3×REH IL-2” display reduced or no binding to IL-2Rα and/or IL-2Rγ polypeptides. In some embodiments, the cells are T cells. In some embodiments, the cells are Treg cells.

In some embodiments, the recombinant cytokine receptor phosphorylates STAT5 through its activation of the JAK1 kinase upon recombinant cytokine receptor activation. In some embodiments, the recombinant cytokine receptor phosphorylates She upon recombinant cytokine receptor activation, thereby activating the downstream PI3K-AKT pathway. In some embodiments, the recombinant cytokine receptor phosphorylates She upon recombinant cytokine receptor activation, thereby activating the downstream Ras/MAPK pathway. In some embodiments, the recombinant cytokine receptor comprises IL-2Rβ tethered to an IL-2 cytokine. In some embodiments, the recombinant cytokine receptor is IL-2Rβ tethered to an IL-2 cytokine.

In some embodiments, the recombinant cytokine receptor has a capability to stimulate STAT5 phosphorylation in cells (e.g., Tregs). STAT5 signaling can be measured, for example, by phosphorylation of STAT5 using any suitable method known in the art. For example, STAT5 phosphorylation can be measured using antibodies specific for the phosphorylated version of these molecules in combination with flow cytometry analysis as described herein.

### Receptor Polypeptide

IL-2 is a class I cytokine. Class I cytokine receptors generally have large extracellular domains (EDs) that include multiple all-β Ig-like domains and Fn3 domains, a single-pass, modular transmembrane domain (TD), and a highly dynamic intracellular domain that transduces the signaling event (see, e.g., Metcalfe, R D et al. (2020), Front Immunol; 11:1424). These domains possess a β-sandwich structure with two anti-parallel β sheets. Two Fn3 domains form the cytokine binding homology region at the domain juncture. Class I cytokine receptor EDs contain a conserved WSXWS (Trp-Ser-X-Trp-Ser, where X is any amino acid) motif that may act to stabilize the receptor, undergo conformational change upon cytokine binding, and can be extensively glycosylated. Class I cytokine receptor chains, including IL-2Rβ, also called IL-2Rβ, are most often found in heterodimers or heterotrimers. For example, IL-2Rβ can be found in a heterodimer with IL-2Rγ_{c} or in a heterotrimer with IL-2Rα and IL-2Rγ_{c} (also called IL-2RA and CD132, respectively), wherein the IL-2 binding affinity for IL-2Rβ/IL-2RB alone is K_{d}˜100 nm, in heterodimeric form K_{d}˜1 nM, and in heterotrimeric form K_{d}˜10 pM (see, e.g., Wang, X et al. (2009), Annu Rev Immunol; 27:29-60, hereby incorporated by reference in its entirety).

The IL-2Rβ polypeptide is primarily expressed in hematopoietic cells, and its involvement in immune cell-mediated immune responses is well described. IL-2Rβ polypeptide interacts with JAK-1 through its C-terminal cytoplasmic domain, which comprises amino acids 240-525. The IL-2Rβ intracellular domain (ID) lacks intrinsic catalytic activity, thus relying upon its constitutive interaction with JAK1 at box 1 and box 3 motifs in its membrane-proximal region to promote IL-2 signal transduction (see, e.g., Wang, X et al. (2009), Annu Rev Immunol; 27:29-60). In activated T cells, IL-2 signal transduction through IL-2Rβ leads to the activation of three pathways: (1) STAT5 phosphorylation via JAK1 kinase, (2) PI3K-AKT pathway activation via Shc phosphorylation, and (3) Ras/MAPK pathway activation via Shc phosphorylation (see, e.g., Ye, C et al. (2018), Signal Transduct Target Ther; 3:2, hereby incorporated by reference in its entirety). Of these three pathways, JAK1/STAT5 signaling is predominant. Activation of the IL-2 pathways across all T cell lineages has been shown to induce T cell proliferation and survival. However, within CD4+IL-2Rα+ Treg cells, IL-2Rβ signaling only engages the JAK1/STAT5 pathway (see, e.g., Bensinger, S J et al. (2010), J Immunol; 172(9):5287-5296, hereby incorporated by reference in its entirety), which is required for Treg suppressive activity (see, e.g., Ye. C et al. (2018), Signal Transduct Target Ther: 3:2). While CD4+, and to a lesser degree CD8+ effector T cells are capable of producing IL-2 to signal their own proliferation and survival after activation. Tregs are not capable of producing IL-2. Therefore, Treg survival relies upon IL-2 produced by effector T cells.

Engaging with the IL-2Rβ chain (i.e., IL-2RB) to generate receptors with varying degrees of affinity for the IL-2 molecule are the polypeptide chains IL-2Rα (i.e., IL-2RA) and IL-2Rγ (i.e., CD132). The IL-2Rα chain, or IL-2RA, is upregulated on active T cells and is constitutively expressed on Tregs. This chain is involved in tolerance regulation and T-cell expansion as it contributes by increasing the binding affinity of IL-2 to the IL-2 receptor (see, e.g., Goudy, K et al. (2013), Clin Immun; 146:248-261, hereby incorporated by reference in its entirety), but the chain is not involved in intracellular signal transduction following the IL-2 binding event (see, e.g., Bezrodnik, L et al. (2014), Clin Exp. Immun.: 175:227-234, hereby incorporated by reference in its entirety). Meanwhile the IL-2Rγ chain, or CD132, can be found in heterodimeric complex with multiple cytokine receptor-α chains (e.g., IL-4Rα, IL-7Rα, IL-15Rα, and IL-21Rα in addition to IL-2Rα and/or IL-2Rβ; see, e.g., Brandt, K et al. (2007), Cytokine Growth Factor Rev; 18:223-232, hereby incorporated by reference in its entirety). The IL-2Rγ chain is important for the formation of high- and intermediate-affinity IL-2 receptors: high-affinity heterotrimers (i.e., a complex of IL-2Rα, IL-2Rβ, and IL-2Rγ) and intermediate-affinity heterodimers (i.e., a complex of IL-2Rβ, and IL-2Rγ), thus indicating that this subunit is important for a functional and sensitive IL-2 receptor (see, e.g., Takeshita, T et al. (1992), Science; 257:379-382), which in turn is critical for T cell biological activity, such as T cell proliferation.

In some embodiments, the recombinant cytokine receptor is able to signal in the absence of exogenous IL-2. In some embodiments, the recombinant cytokine receptors provided herein comprises an active IL-2Rβ polypeptide with the wild-type extracellular, transmembrane, and intracellular domains. In some embodiments, the intracellular domain of the recombinant cytokine receptors provided herein is able to engage in downstream signal transduction. In some embodiments, the IL-2Rβ intracellular domain phosphorylates STAT5 through its activation of the JAK1 kinase upon IL-2Rβ activation. In some embodiments, the IL-2Rβ intracellular domain phosphorylates Shc upon IL-2Rβ receptor activation, thereby activating the downstream PI3K-AKT pathway. In some embodiments, the IL-2Rβ intracellular domain phosphorylates Shc upon IL-2Rβ activation, thereby activating the downstream Ras/MAPK pathway. In some embodiments, the IL-2Rβ intracellular domain phosphorylates STAT5 through its activation of the JAK1 kinase upon recombinant cytokine receptor activation. In some embodiments, the IL-2Rβ intracellular domain phosphorylates She upon recombinant cytokine receptor activation, thereby activating the downstream PI3K-AKT pathway. In some embodiments, the IL-2Rβ intracellular domain phosphorylates She upon recombinant cytokine receptor activation, thereby activating the downstream Ras/MAPK pathway. In some embodiments, the recombinant cytokine receptor comprises IL-2Rβ tethered by a polypeptide linker to wild-type IL-2. mutant 3×IL-2, mutant REH IL-2, or mutant 3×+REH IL-2. In some embodiments, the recombinant cytokine receptor is wild-type IL-2/IL-2Rβ (“IL-2”), mutant 3×IL-2/IL-2Rβ (“3×”), mutant REH IL-2/IL-2Rβ (“REH”), or mutant 3×+REH IL-2/IL-2Rβ (“3×REH”). In some embodiments, the recombinant cytokine receptor comprises an IL-2Rβ polypeptide, a polypeptide linker, and an IL-2 cytokine. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. Thus, in some embodiments, there is provided a recombinant cytokine receptor comprising: (I) an IL-2 cytokine; (II) the polypeptide linker; and (ill) IL-2Rβ. In some embodiments, the recombinant cytokine receptor comprises the amino acid sequence selected from the group consisting of SEQ ID NOs:1-3 and 11. In some embodiments, the IL-2 cytokine comprises the amino acid sequence selected from the group consisting of SEQ ID NOs:4-6 and 12. In some embodiments, there is provided a recombinant cytokine receptor comprising: (I) a wild-type IL-2 cytokine, (II) a polypeptide linker, (III) an extracellular domain, (IV) a transmembrane domain, and (V) an intracellular domain. In some embodiments, the recombinant cytokine receptor does not comprise a TCR activation domain or a costimulatory domain (e.g., a CD3 or a CD28 activation or costimulatory domain, such as a CD28 signaling domain). In some embodiments, the recombinant cytokine receptor comprises the amino acid sequence set forth in SEQ ID NO:1. In some embodiments, the IL-2 cytokine comprises the amino acid sequence set forth in SEQ ID NO:4. In some embodiments, there is provided a recombinant cytokine receptor comprising: (1) a 3×IL-2 cytokine, (II) a polypeptide linker, (III) an IL-2 receptor extracellular domain, (IV) a transmembrane domain, and (V) an IL-2 receptor intracellular domain. In some embodiments, the recombinant cytokine receptor comprises the amino acid sequence set forth in SEQ ID NO:2. In some embodiments, the IL-2 cytokine comprises the amino acid sequence set forth in SEQ ID NO:5. In some embodiments, there is provided a recombinant cytokine receptor comprising: (I) an REH IL-2 cytokine, (II) a polypeptide linker, (III) an IL-2 receptor extracellular domain, (IV) a transmembrane domain, and (V) an IL-2 receptor intracellular domain. In some embodiments, the recombinant cytokine receptor comprises the amino acid sequence set forth in SEQ ID NO:3. In some embodiments, the IL-2 cytokine comprises the amino acid sequence set forth in SEQ ID NO:6. In some embodiments, there is provided a recombinant cytokine receptor comprising: (I) a 3×REH IL-2 cytokine, (II) a polypeptide linker, (III) an IL-2 receptor extracellular domain, (IV) a transmembrane domain, and (V) an IL-2 receptor intracellular domain. In some embodiments, the recombinant cytokine receptor comprises the amino acid sequence set forth in SEQ ID NO:11. In some embodiments, the IL-2 cytokine comprises the amino acid sequence set forth in SEQ ID NO:12. In some embodiments, the recombinant cytokine receptors comprise an active IL-2Rβ polypeptide tethered to a mutated IL-2 cytokine comprising one or more amino acid substitutions selected from the group consisting of L18R, Q22E, Q126H, R38D, K43E, and E61R. In some embodiments, the mutated IL-2 cytokine may comprise additional amino acid substitutions. In some embodiments, the IL-2Rβ polypeptide further comprises: (a) an IL-2 receptor extracellular domain (ED), (b) a transmembrane domain (TD), and (c) an IL-2 receptor intracellular domain (ID). In some embodiments, the ED of the IL-2Rβ polypeptide comprises an amino acid sequence of SEQ ID NO:13. In some embodiments, the full-length IL-2Rβ polypeptide comprises an amino acid sequence of SEQ ID NO:7. In some embodiments, the full-length IL-2Rβ polypeptide comprises an amino acid sequence comprising at least 80%, at least 85%, at least 90%, at least 95%, at least 96%, at least 97%, at least 98%, at least 99% or 100% sequence identity with the amino acid sequence set forth in SEQ ID NO:7. In some embodiments, an IL-2 cytokine comprising one or more amino acid substitutions selected from the group consisting of L18R, Q22E, Q126H, R38D, K43E, and E61R displays reduced or no binding affinity for IL-2Rα and/or IL-2Rγ but does not reduce or eliminate binding to IL-2Rβ.

In some embodiments, the recombinant cytokine receptor comprises the wild-type IL-2 cytokine. In some embodiments the wild-type IL-2 cytokine comprises the amino acid sequence set forth in SEQ ID NO:4. In some embodiments, the wild-type IL-2 cytokine comprises an amino acid sequence comprising at least about 80%, at least about 85%, at least about 90%, at least about 95%, at least about 96%, at least about 97%, at least about 98%, at least about 99% or about 100% to SEQ ID NO:4. In some embodiments, the recombinant cytokine receptor comprises in N-terminal to C-terminal direction: (I) the wild-type IL-2 cytokine, (II) a polypeptide linker, (III) an extracellular domain, (IV) a transmembrane domain, and (V) an intracellular domain. In some embodiments, the recombinant cytokine receptor comprises in N-terminal to C-terminal direction: (I) the wild-type IL-2 cytokine, (II) a polypeptide linker. (III) an IL-2 receptor extracellular domain, (IV) a transmembrane domain, and (V) an IL-2 receptor intracellular domain. In some embodiments, the recombinant cytokine receptor does not comprise a TCR activation domain or a costimulatory domain (e.g., a CD3 or a CD28 activation or costimulatory domain, such as a CD28 signaling domain). In some embodiments, the extracellular comprises an amino acid sequence of SEQ ID NO:13. In some embodiments, the extracellular comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane of the IL-2Rβ polypeptide comprises an amino acid sequence of SEQ ID NO:14. In some embodiments, the TD comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the ID comprises an amino acid sequence of SEQ ID NO:15. In some embodiments, the ID comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F.

In some embodiments, the recombinant cytokine receptor comprises the mutant 3×IL-2 cytokine. In some embodiments, the mutant 3×IL-2 cytokine comprises the amino acid sequence set forth in SEQ ID NO:5. In some embodiments, the mutant 3×IL-2 cytokine comprises an amino acid sequence comprising at least about 80%, at least about 85%, at least about 90%, at least about 95%, at least about 96%, at least about 97%, at least about 98%, at least about 99% or about 100% to SEQ ID NO:5. In some embodiments, the recombinant cytokine receptor comprises in N-terminal to C-terminal direction: (I) the mutant 3×IL-2 cytokine, (II) a polypeptide linker, (III) an IL-2 receptor extracellular domain, (IV) a transmembrane domain, and (V) an IL-2 receptor intracellular domain. In some embodiments, the extracellular domain comprises an amino acid sequence of SEQ ID NO:13. In some embodiments, the ED comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain comprises an amino acid sequence of SEQ ID NO:14. In some embodiments, the TD comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain comprises an amino acid sequence of SEQ ID NO:15. In some embodiments, the ID comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, the full-length IL-2Rβ polypeptide comprises an amino acid sequence of SEQ ID NO:7. In some embodiments, the full-length IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:7. In some embodiments, the recombinant cytokine receptor comprises an IL-2 cytokine comprising one or more amino acid substitutions selected from the group consisting of R38D, K43E, and E61R. In some embodiments, the recombinant cytokine receptor comprises an IL-2 cytokine comprising the amino acid substitutions R38D, K43E, and E61R. In some embodiments, the, recombinant cytokine receptor comprises an IL-2 cytokine comprising the amino acid substitutions R38D, K43E, and E61R and one or more additional amino acid substitutions. In some embodiments, an IL-2 cytokine comprising one or more amino acid substitutions selected from the group consisting of R38D, K43E, and E61R displays reduced or no binding affinity for IL-2Rα and/or IL-2Rγ but does not reduce or eliminate binding to IL-2Rβ. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F.

In some embodiments, the recombinant cytokine receptor comprises the mutant REH IL-2 molecule. In some embodiments, the mutant REH IL-2 molecule comprises the amino acid sequence set forth in SEQ ID NO:6. In some embodiments, the mutant REH IL-2 molecule comprises an amino acid sequence comprising at least about 80%, at least about 85%, at least about 90%, at least about 95%, at least about 96%, at least about 97%, at least about 98%, at least about 99% or about 100% to SEQ ID NO:6. In some embodiments, the recombinant cytokine receptor comprises in N-terminal to C-terminal direction: (I) the mutant REH IL-2 molecule, (II) a polypeptide linker, (III) an IL-2 receptor extracellular domain, (IV) a transmembrane domain, and (V) an IL-2 receptor intracellular domain. In some embodiments, the full-length IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:7. In some embodiments, the recombinant cytokine receptor comprises an IL-2 cytokine comprising one or more amino acid substitutions selected from the group consisting of L18R, Q22E, and Q126H. In some embodiments, the recombinant cytokine receptor comprises an IL-2 cytokine comprising the amino acid substitutions L18R, Q22E, and Q126H. In some embodiments, the, recombinant cytokine receptor comprises an IL-2 cytokine comprising the amino acid substitutions L18R, Q22E, and Q126H and one or more additional amino acid substitutions. In some embodiments, an IL-2 cytokine comprising one or more amino acid substitutions selected from the group consisting of L18R, Q22E, and Q126H displays reduced or no binding affinity for IL-2Rα and/or IL-2Rγ but does not reduce or eliminate binding to IL-2Rβ. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F.

In some embodiments, the recombinant cytokine receptor comprises the mutant 3×REH IL-2 cytokine. In some embodiments, the mutant 3×REH IL-2 cytokine comprises the amino acid sequence set forth in SEQ ID NO:12. In some embodiments, the mutant 3×REH IL-2 cytokine comprises an amino acid sequence comprising at least about 80%, at least about 85%, at least about 90%, at least about 95%, at least about 96%, at least about 97%, at least about 98%, at least about 99% or about 100% to SEQ ID NO:12. In some embodiments, the recombinant cytokine receptor comprises in N-terminal to C-terminal direction: (I) the 3×REH IL-2 cytokine, (II) a polypeptide linker, (III) an IL-2 receptor extracellular domain, (IV) a transmembrane domain, and (V) an IL-2 receptor intracellular domain. In some embodiments, the full-length IL-2Rβ polypeptide comprises an amino acid sequence of SEQ ID NO:7. In some embodiments, the full-length IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:7. In some embodiments, the recombinant cytokine receptor comprises an IL-2 cytokine comprising one or more amino acid substitutions selected from the group consisting of L18R, Q22E, Q126H, R38D, K43E, and E61R. In some embodiments, the recombinant cytokine receptor comprises an IL-2 cytokine comprising the amino acid substitutions L18R, Q22E, Q126H, R38D, K43E, and E61R. In some embodiments, the, recombinant cytokine receptor comprises an IL-2 cytokine comprising the amino acid substitutions L18R, Q22E, Q126H, R38D, K43E, and E61R and one or more additional amino acid substitutions. In some embodiments, an IL-2 cytokine comprising one or more amino acid substitutions selected from the group consisting of L18R, Q22E, Q126H, R38D, K43E, and E61R displays reduced or no binding affinity for IL-2R, and/or IL-2Rγ but does not reduce or eliminate binding to IL-2Rβ. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F.

In some embodiments, the recombinant cytokine receptor comprising the aforementioned IL-2 cytokine-tethered IL-2Rβ polypeptide is expressed in cells. In some embodiments, the recombinant cytokine receptor comprising the aforementioned IL-2 cytokine-tethered IL-2Rβ polypeptide is expressed in immune cells. In some embodiments, the immune cells are T cells. In some embodiments, the immune cells are Treg cells.

In some embodiments, the recombinant cytokine receptor comprising the aforementioned IL-2 cytokine-tethered IL-2Rβ polypeptide is expressed in Tregs. In some embodiments, the Treg cells are CD4+, CD25+, and CD127lo. In some embodiments, the Tregs that express the recombinant cytokine receptor comprising the aforementioned IL-2 cytokine-tethered IL-2Rβ polypeptide also express FOXP3 and HELIOS. In some embodiments, the Tregs that express the recombinant cytokine receptor comprising the aforementioned IL-2 cytokine-tethered IL-2Rβ polypeptide also express high levels of FOXP3 and HELIOS. In some embodiments, the Tregs expressing the recombinant cytokine receptor comprising the aforementioned IL-2 cytokine-tethered IL-2Rβ polypeptide survive and proliferate in the absence of exogenous IL-2. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor comprising the aforementioned IL-2 cytokine-tethered IL-2Rβ polypeptide remain viable in vitro 2-23 days after transduction. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor comprising the aforementioned IL-2 cytokine-tethered IL-2Rβ polypeptide persist in vivo for at least about 3 days or longer. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor comprising the aforementioned IL-2 cytokine-tethered IL-2Rβ polypeptide persist in vivo for at least about 14 days or longer. In some embodiments, at least about 60% (such as at least about any of 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%) of the Tregs transduced with the recombinant cytokine receptor comprising the aforementioned IL-2 cytokine-tethered IL-2Rβ polypeptide persist in vivo for at least about 23 days or longer. In some embodiments, the recombinant cytokine receptor comprising the aforementioned IL-2 cytokine-tethered IL-2Rβ polypeptide expressed in Tregs transduces intracellular IL-2 signaling within the Tregs. In some embodiments, the recombinant cytokine receptor comprising the aforementioned IL-2 cytokine-tethered IL-2Rβ polypeptide is able to signal in the absence of exogenous IL-2. In some embodiments, the recombinant cytokine receptor comprising the aforementioned IL-2 cytokine-tethered IL-2Rβ polypeptide is able to signal in the absence of exogenous interleukin cytokine. In some embodiments, signaling through the recombinant cytokine receptor comprising the aforementioned IL-2 cytokine-tethered IL-2Rβ polypeptide expressed in the Tregs induces phosphorylation of STAT5. In some embodiments, the recombinant cytokine receptor does not activate IL-2 signaling on a cell that does not comprise the recombinant cytokine receptor. In some embodiments, the recombinant cytokine receptor does not activate IL-2 signaling on Tregs that do not comprise the recombinant cytokine receptor.

In some embodiments the recombinant cytokine receptor does not activate signaling of a different IL-2 receptor, such as IL-2Rα and/or IL-2Rγ.

Stable imprinting of the suppressive phenotype has been suggested to be mediated by the Treg-specific de-methylated region (TSDR, also called CNS2) in the FOXP3 gene, an epigenetic switch region which is selectively activated by DNA de-methylation in Tregs and sustains FOXP3 protein expression by epigenetic regulation. In some embodiments, the recombinant cytokine receptor allows the transduced Treg cell to proliferate without exogenous IL-2. In some embodiments, the relative amount of Treg cells in a composition comprising a population of Treg cells transduced with the recombinant cytokine receptor increases over time. In some embodiments, the population of Treg cells transduced with the recombinant cytokine receptor grown without IL-2 contains a similar number of viable cells compared to a composition comprising a population of the same Treg cells not transduced with the recombinant cytokine receptor grown with IL-2. In some embodiments, at least 80% of the population of Treg cells transduced with the recombinant cytokine receptor maintain FOXP3 and/or HELIOS expression 14 days after transduction. In some embodiments, at least 80% of the cells in the population of Treg cells transduced with the recombinant cytokine maintain FOXP3 and/or HELIOS expression 23 days after transduction.

In some embodiments, the population of Treg cells transduced with the recombinant cytokine receptor expands at least two-fold more than a population of the same Treg cells not transduced with the recombinant cytokine receptor. In some embodiments, the population of Treg cells transduced with the recombinant cytokine receptor expands at least two-fold, and wherein the population of Treg cells transduced with the recombinant cytokine receptor maintains expression of least one Treg marker selected from the group consisting of CD25+, FOXP3, and HELIOS.

In some embodiments, the relative amount of Treg cells in a composition comprising a population of Treg cells transduced with the recombinant cytokine receptor increases over time when cultured without IL-2.

In some embodiments, the Tregs expressing the recombinant cytokine receptor comprising the aforementioned IL-2 cytokine-tethered IL-2Rβ polypeptide are capable of suppressing the activity of effector T cells in the absence of exogenous IL-2 to at least the same degree as or more than wild-type Tregs cultured with exogenous IL-2. In some embodiments, the suppressed effector T cells are CD4+ effector T cells. In some embodiments, the suppressed effector T cells are CD8+ effector T cells. In some embodiments. Tregs that express the recombinant cytokine receptor comprising the aforementioned IL-2 cytokine-tethered IL-2Rβ polypeptide also express chimeric antigen receptors (CARs).

### Tethered Interleukin-2 Cytokine and Polypeptide Linkers

As described above, IL-2 is a class I cytokine that is capable of binding IL-2Rα, IL-2Rβ, and IL-2Rγ at different binding affinities for the generation of IL-2 receptor complexes that display varying sensitivities to the presence of IL-2. Thus, in some embodiments, the recombinant cytokine receptor comprises an IL-2 cytokine wherein the IL-2 cytokine is a wild-type IL-2 molecule. In some embodiments, the recombinant cytokine receptor comprises an IL-2 cytokine comprising at least one or more amino acid substitutions at positions selected from amino acid positions 18, 22, 126, 38, 43, 61, 15, 16, 19, 20, 22, 23, and 81. In some embodiments, the recombinant cytokine receptor comprises an IL-2 cytokine wherein the IL-2 cytokine comprises one or more amino acid substitutions selected from the group consisting of L18R, Q22E, Q126H, R38D, K43E, E61R, E15S, H16Q, L19V, D20L, Q22K, M23Q, M23A, and R81D. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the IL-2 cytokine is mammalian. In some embodiments, the IL-2 cytokine is human. In some embodiments, the recombinant cytokine receptor comprises an IL-2 cytokine that does not activate IL-2 signaling on cells that do not comprise the recombinant cytokine receptor. In some embodiments, the recombinant cytokine receptor comprises an IL-2 cytokine that does not activate IL-2 signaling of a different IL-2 receptor. In some embodiments, the recombinant cytokine receptor does not comprise a T cell receptor activation domain or a T cell costimulatory domain, optionally wherein the T cell receptor activation domain or T cell costimulatory domain is a CD28 signaling domain. In some embodiments, the recombinant cytokine receptor comprises in N-terminal to C-terminal direction: (I) an IL-2 cytokine, (II) a polypeptide linker, (III) an extracellular domain, (IV) a transmembrane domain, and (V) an intracellular domain. In some embodiments, the IL-2 cytokine that is tethered by a polypeptide linker to the IL-2Rβ polypeptide of the recombinant cytokine receptor comprises any of the SEQ ID NOs:4-6 and 12. In some embodiments, the IL-2 cytokine comprises an amino acid sequence comprising at least about 80%, at least about 85%, at least about 90%, at least about 95%, at least about 96%, at least about 97%, at least about 98%, at least about 99% or about 100% sequence identity with the amino acid sequence selected from the group consisting of SEQ ID NOs:4-6 and 12. In some embodiments, there is provided a recombinant cytokine receptor comprising from N to C terminus: (I) an IL-2 cytokine comprising an amino acid sequence selected from the group consisting of SEQ ID NOs:4-6 and 12, (I) a polypeptide linker, (III) an IL-2Rβ extracellular domain, (IV) a transmembrane domain, and (V) an IL-2Rβ intracellular domain. In some embodiments, the IL-2 tethered IL-2Rβ recombinant cytokine receptor comprises an amino acid sequence comprising at least about 80%, at least about 85%, at least about 90%, at least about 95%, at least about 96%, at least about 97%, at least about 98%, at least about 99% or about 100% sequence identity with the amino acid sequence selected from the group consisting of SEQ ID NOs:1-3 and 11, wherein the cytokine receptor comprises an IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence selected from the group consisting of SEQ ID NOs:4-6 and 12, and wherein the polypeptide linker having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:9. In some embodiments, the ED of the IL-2Rβ polypeptide comprises an amino acid sequence of SEQ ID NO:13. In some embodiments, the ED of the IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the TD of the IL-2Rβ polypeptide comprises an amino acid sequence of SEQ ID NO:14. In some embodiments, the TD of the IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the ID of the IL-2Rβ polypeptide comprises an amino acid sequence of SEQ ID NO:15. In some embodiments, the ID of the IL-2Rβ polypeptide comprises an amino acid sequence of SEQ ID NO:15. In some embodiments, the full-length IL-2Rβ polypeptide comprises an amino acid sequence of SEQ ID NO:7. In some embodiments, the full-length IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:7.

In some embodiments, the recombinant cytokine receptor comprises a wild-type IL-2 cytokine tethered to the IL-2Rβ polypeptide. In some embodiments, the recombinant cytokine receptor comprises a wild-type IL-2 cytokine tethered to the N-terminus of the extracellular domain of the full-length IL-2Rβ polypeptide. In some embodiments, the recombinant cytokine receptor comprises in N-terminal to C-terminal direction: (I) wild-type IL-2 cytokine, (II) a polypeptide linker, (III) an extracellular domain, (IV) a transmembrane domain, and (V) an intracellular domain. In some embodiments, the recombinant cytokine receptor does not comprise a TCR activation domain or a costimulatory domain (e.g., a CD3 or a CD28 activation or costimulatory domain, such as a CD28 signaling domain). In some embodiments, wild-type IL-2 comprises the amino acid sequence set forth in SEQ ID NO:4. In some embodiments, wild-type IL-2 comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:4. In some embodiments, there is provided a recombinant cytokine receptor comprising from N to C terminus: (I) an IL-2 cytokine comprising the amino acid sequence of SEQ ID NO:4, (II) a polypeptide linker, (III) an IL-2Rβ extracellular domain, (IV) a transmembrane domain, and (V) an IL-2Rβ intracellular domain. In some embodiments, there is provided a recombinant cytokine receptor comprising from N to C terminus: (I) an IL-2 cytokine comprising the amino acid sequence of SEQ ID NO:4, (II) a polypeptide linker, (III) an IL-2Rβ extracellular domain, (IV) a transmembrane domain, and (V) an IL-2Rβ intracellular domain, wherein the recombinant cytokine receptor does not comprise a T cell receptor activation domain or a T cell costimulatory domain, optionally wherein the T cell receptor activation domain or T cell costimulatory domain is a CD28 signaling domain. In some embodiments, the wild-type IL-2 tethered IL-2Rβ recombinant cytokine receptor comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:1, wherein the cytokine receptor comprises a wild-type IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:4, and wherein the polypeptide linker having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:9. In some embodiments, the extracellular domain of the IL-2Rβ polypeptide comprises an amino acid sequence of SEQ ID NO:13. In some embodiments, the ED of the IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain of the IL-2Rβ polypeptide comprises an amino acid sequence of SEQ ID NO:14. In some embodiments, the transmembrane domain of the IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain of the IL-2Rβ polypeptide comprises an amino acid sequence of SEQ ID NO:15. In some embodiments, the ID of the IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, the full-length IL-2Rβ polypeptide comprises an amino acid sequence of SEQ ID NO:7. In some embodiments, the full-length IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:7. In some embodiments, the wild-type IL-2 cytokine that is tethered to the IL-2Rβ polypeptide also binds to IL-2Rα and/or IL-2Rγ polypeptides that are complexed with the IL-2 tethered IL-2Rβ polypeptide. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F.

In some embodiments, the recombinant cytokine receptor comprises an IL-2 cytokine comprising at least one or more amino acid substitutions at positions selected from amino acid positions 18, 22, 126, 38, 43 and 61. In some embodiments, the recombinant cytokine receptor comprises an IL-2 cytokine wherein the IL-2 cytokine comprises one or more amino acid substitutions selected from the group consisting of R38D, K43E, and E61R. In some embodiments, the recombinant cytokine receptor comprises an IL-2 cytokine wherein the IL-2 cytokine comprises amino acid substitutions R38D, K43E, and E61R (“3×IL-2”). In some embodiments, the recombinant cytokine receptor comprises an IL-2 cytokine wherein the IL-2 cytokine comprises amino acid substitutions R38D, K43E, and E61R and one or more additional amino acid substitutions. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the recombinant cytokine receptor comprises a mutant 3×IL-2 cytokine tethered to the IL-2Rβ polypeptide. In some embodiments, the recombinant cytokine receptor comprises a mutant 3×IL-2 cytokine tethered to the N-terminus of the extracellular domain of the full-length IL-2Rβ polypeptide. In some embodiments, the recombinant cytokine receptor comprises in N-terminal to C-terminal direction: (I) the 3×IL-2 cytokine; (II) a polypeptide linker, (III) an extracellular domain, (IV) a transmembrane domain, and (V) an intracellular domain. In some embodiments, the recombinant cytokine receptor comprises in N-terminal to C-terminal direction: (I) the 3×IL-2 cytokine, (II) the polypeptide linker; and (III) the full-length IL-2Rβ polypeptide. In some embodiments, mutant 3×IL-2 comprises the amino acid sequence set forth in SEQ ID NO:5. In some embodiments, mutant 3×IL-2 comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:5. In some embodiments, there is provided a recombinant cytokine receptor comprising from N to C terminus: (I) an IL-2 cytokine comprising the amino acid sequence of SEQ ID NO:5, (II) a polypeptide linker, (III) an IL-2Rβ extracellular domain, (IV) a transmembrane domain, and (V) an IL-2Rβ intracellular domain. In some embodiments, the mutant 3×IL-2 tethered IL-2Rβ recombinant cytokine receptor comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:2, wherein the cytokine receptor comprises a mutant 3×IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:5, and wherein the polypeptide linker having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:9. In some embodiments, the ED of the IL-2Rβ polypeptide comprises an amino acid sequence of SEQ ID NO:13. In some embodiments, the ED of the IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the TD of the IL-2Rβ polypeptide comprises an amino acid sequence of SEQ ID NO:14. In some embodiments, the TD of the IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the ID of the IL-2Rβ polypeptide comprises an amino acid sequence of SEQ ID NO:15. In some embodiments, the ID of the IL-2Rβ polypeptide comprises an amino acid sequence comprising at least about 80%, at least about 85%, at least about 90%, at least about 95%, at least about %%, at least about 97%, at least about 98%, at least about 99% or about 100% sequence identity with the amino acid sequence set forth in SEQ ID NO:15. In some embodiments, the full-length IL-2Rβ polypeptide comprises an amino acid sequence of SEQ ID NO:7. In some embodiments, the full-length IL-2R polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:7. In some embodiments, the mutant 3×IL-2 cytokine displays reduced or no binding to IL-2Rα and/or IL-2Rγ but does not reduce or eliminate binding to IL-2Rβ.

In some embodiments, the recombinant cytokine receptor comprises an IL-2 cytokine wherein the IL-2 cytokine comprises one or more amino acid substitutions selected from the group consisting of L18R, Q22E, and Q126H. In some embodiments, the recombinant cytokine receptor comprises an IL-2 cytokine wherein the IL-2 cytokine comprises amino acid substitutions L18R, Q22E, and Q126H (“REH IL-2”). In some embodiments, the recombinant cytokine receptor comprises an IL-2 cytokine wherein the IL-2 cytokine comprises amino acid substitutions L18R, Q22E, and Q126H and one or more additional amino acid substitutions. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the recombinant cytokine receptor comprises a mutant REH IL-2 cytokine tethered to the IL-2Rβ polypeptide. In some embodiments, the recombinant cytokine receptor comprises a mutant REH IL-2 cytokine tethered to the N-terminus of the extracellular domain of the full-length IL-2Rβ polypeptide chain. In some embodiments, the recombinant cytokine receptor comprises in N-terminal to C-terminal direction: (I) the REH IL-2 cytokine, (II) a polypeptide linker, (III) an extracellular domain, (IV) a transmembrane domain, and (V) an intracellular domain. In some embodiments, the recombinant cytokine receptor comprises in N-terminal to C-terminal direction: (I) the REH IL-2 cytokine; (II) the polypeptide linker; and (III) the full-length IL-2Rβ polypeptide. In some embodiments, mutant REH IL-2 comprises the amino acid sequence set forth in SEQ ID NO:6. In some embodiments, mutant REH IL-2 comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:6. In some embodiments, there is provided a recombinant cytokine receptor comprising from N to C terminus: (I) an IL-2 cytokine comprising the amino acid sequence of SEQ ID NO:6, (II) a polypeptide linker, (III) an IL-2Rβ extracellular domain, (IV) a transmembrane domain, and (V) an IL-2Rβ intracellular domain. In some embodiments, the mutant REH IL-2 tethered IL-2Rβ recombinant cytokine receptor comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:3, wherein the cytokine receptor comprises a mutant REH IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:6, and wherein the polypeptide linker having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:9. In some embodiments, the ED of the IL-2Rβ polypeptide comprises an amino acid sequence of SEQ ID NO:13. In some embodiments, the ED of the IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the TD of the IL-2Rβ polypeptide comprises an amino acid sequence of SEQ ID NO:14. In some embodiments, the TD of the IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the ID of the IL-2Rβ polypeptide comprises an amino acid sequence of SEQ ID NO:15. In some embodiments, the ID of the IL-2Rβ polypeptide comprises an amino acid sequence comprising at least about 80%, at least about 85%, at least about 90%, at least about 95%, at least about %%, at least about 97%, at least about 98%, at least about 99% or about 100% sequence identity with the amino acid sequence set forth in SEQ ID NO:15. In some embodiments, the full-length IL-2Rβ polypeptide comprises an amino acid sequence of SEQ ID NO:7. In some embodiments, the full-length IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:7. In some embodiments, the mutant REH IL-2 cytokine displays reduced or no binding to IL-2Rα and/or IL-2Rγ but does not reduce or eliminate binding to IL-2Rβ.

In some embodiments, the recombinant cytokine receptor comprises an IL-2 cytokine wherein the IL-2 cytokine comprises one or more amino acid substitutions selected from the group consisting of L18R, Q22E, Q126H, R38D, K43E, and E61R. In some embodiments, the recombinant cytokine receptor comprises an IL-2 cytokine wherein the IL-2 cytokine comprises amino acid substitutions L18R, Q22E, Q126H, R38D, K43E, and E61R (“3×REH IL-2”). In some embodiments, the recombinant cytokine receptor comprises an IL-2 cytokine wherein the IL-2 cytokine comprises amino acid substitutions L18R, Q22E, Q126H, R38D, K43E, and E61R and one or more additional amino acid substitutions. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the recombinant cytokine receptor comprises a mutant 3×REH IL-2 cytokine tethered to the IL-2Rβ polypeptide. In some embodiments, the recombinant cytokine receptor comprises a mutant 3×REH IL-2 cytokine tethered to the N-terminus of the extracellular domain of the full-length IL-2Rβ polypeptide chain. In some embodiments, the recombinant cytokine receptor comprises in N-terminal to C-terminal direction: (I) the 3×REH IL-2 cytokine, (II) a polypeptide linker, (III) an extracellular domain, (IV) a transmembrane domain, and (V) an intracellular domain. In some embodiments, the recombinant cytokine receptor comprises in N-terminal to C-terminal direction: (I) the 3×REH IL-2 cytokine; (II) the polypeptide linker; and (III) the full-length IL-2Rβ polypeptide. In some embodiments, the mutant 3×REH IL-2 comprises the amino acid sequence set forth in SEQ ID NO:12. In some embodiments, the mutant 3×REH IL-2 comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:12. In some embodiments, there is provided a recombinant cytokine receptor comprising from N to C terminus: (I) an IL-2 cytokine comprising the amino acid sequence of SEQ ID NO:12, (II) a polypeptide linker, (III) an IL-2Rβ extracellular domain, (IV) a transmembrane domain, and (V) an IL-2Rβ intracellular domain. In some embodiments, the mutant 3×REH IL-2 tethered IL-2Rβ recombinant cytokine receptor comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:11. wherein the cytokine receptor comprises a mutant 3×REH IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:11, and wherein the polypeptide linker having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:9. In some embodiments, the ED of the IL-2Rβ polypeptide comprises an amino acid sequence of SEQ ID NO:13. In some embodiments, the ED of the IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the TD of the IL-2Rβ polypeptide comprises an amino acid sequence of SEQ ID NO:14. In some embodiments, the TD of the IL-2Rβ polypeptide comprises an amino acid sequence comprising at least about 80%, at least about 85%, at least about 90%, at least about 95%, at least about %%, at least about 97%, at least about 98%, at least about 99% or about 100% sequence identity with the amino acid sequence set forth in SEQ ID NO:14. In some embodiments, the ID of the IL-2Rβ polypeptide comprises an amino acid sequence of SEQ ID NO:15. In some embodiments, the ID of the IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, the full-length IL-2Rβ polypeptide comprises an amino acid sequence of SEQ ID NO:7. In some embodiments, the full-length IL-2Rβ polypeptide comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:7. In some embodiments, the mutant 3×REH IL-2 cytokine displays reduced binding affinity to IL-2Rα and/or IL-2Rγ. In some embodiments, the mutant 3×REH IL-2 cytokine displays no binding to IL-2Rα and/or IL-2Rγ but does not reduce or eliminate binding to IL-2Rβ.

The polypeptide linkers can be of any length. In some embodiments, the polypeptide linker is from about 1 amino acid to about 10 amino acids long, from about 2 amino acids to about 15 amino acids long, from about 3 amino acids to about 12 amino acids long, from about 4 amino acids to about 10 amino acids long, from about 5 amino acids to about 9 amino acids long, from about 6 amino acids to about 8 amino acids long, from about 1 amino acid to about 20 amino acids long, from about 21 amino acids to about 30 amino acids long, from about 1 amino acid to about 30 amino acids long, from about 2 amino acids to about 20 amino acids long, from about 10 amino acids to about 30 amino acids long, from about 2 amino acids to about 19 amino acids long, from about 2 amino acids to about 18 amino acids long, from about 2 amino acids to about 17 amino acids long, from about 2 amino acids to about 16 amino acids long, from about 2 amino acids to about 10 amino acids long, from about 2 amino acids to about 14 amino acids long, from about 2 amino acids to about 13 amino acids long, from about 2 amino acids to about 12 amino acids long, from about 2 amino acids to about 11 amino acids long, from about 2 amino acids to about 9 amino acids long, from about 2 amino acids to about 8 amino acids long, from about 2 amino acids to about 7 amino acids long, from about 2 amino acids to about 6 amino acids long, from about 2 amino acids to about 5 amino acids long, from about 2 amino acids to about 4 amino acids long, or from about 2 amino acids to about 3 amino acids long. In some embodiments, the peptide linker is any of 1, 2, 3, 4, 5, 6, 7, 8, 9, 10, 11, 12, 13, 14, 15, 16, 17, 18, 19, or 20 amino acids long. In some embodiments, the polypeptide linker is any of 21, 22, 23, 24, 25, 26, 27, 28, 29, or 30 amino acids long. For example, in some embodiments, the polypeptide linker is about 5 amino acids long. In some embodiments, the N-terminus of the polypeptide linker is covalently linked to the C-terminus of the IL-2 cytokine, and the C-terminus of the polypeptide linker is covalently linked to the N-terminus of the IL-2Rβ polypeptide.

In some embodiments, the IL-2 receptor extracellular domain is tethered to the IL-2 cytokine by a polypeptide linker. A polypeptide linker can have a naturally occurring sequence or a non-naturally occurring sequence. For example, a sequence derived from the hinge region of a heavy chain only antibody can be used as a linker. See, for example, WO1996/34103. In some embodiments, the linker is a flexible linker. In some embodiments, the exemplary flexible linker is (GGGGS)ₙ (SEQ ID NO:20) where n is an integer of at least one, for example (GGGGS)ₙ (SEQ ID NO:20) wherein n is 1-5. Glycine and glycine-serine polymers are relatively unstructured, and therefore may be able to serve as a neutral tether between components. Glycine accesses significantly more phi-psi space than even alanine and is much less restricted than residues with longer side chains (see Scheraga, Rev. Computational Chem. 11173-142 (1992)). Thus, an exemplary flexible linker includes, but is not limited to. Gly-Gly-Gly-Gly-Ser-Gly-Gly-Gly-Gly-Ser (SEQ ID NO:19). In some embodiments, the linker comprises the amino acid sequence GGGGS (SEQ ID NO: 16). In some embodiments, the linker comprises the amino acid sequence GGGGSGGGGSGGGGS (SEQ ID NO:17). In some embodiments, the linker comprises the amino acid sequence SGGGSGGGGSGGGGSGGGGSGGGSLQ (SEQ ID NO:18). The ordinarily skilled artisan will recognize that design of a recombinant cytokine receptor tethered to human IL-2 cytokine can include linkers that are all or partially flexible, such that the linker can include a flexible linker portion as well as one or more portions that confer less flexible structure to provide a desired recombinant cytokine receptor structure.

In some embodiments, the linker between the IL-2 cytokine and the IL-2Rβ polypeptide is a stable, non-cleavable linker. In some embodiments, the linker between the IL-2 cytokine and the IL-2Rβ polypeptide is not cleavable by a protease. In some embodiments, the linker between the IL-2 cytokine and the IL-2Rβ polypeptide is a flexible linker.

### II. Cells

The disclosure provides for an adoptive cellular immunotherapy composition comprising a genetically modified cell preparation as described herein, e.g., genetically modified immune cells, e.g., lymphocytes. These cells are, for example, multipotent cells such as hematopoietic stem cells, various progenitor or precursor cells of hematopoietic lineages, and various immune cells (e.g., human autologous or allogeneic T. natural killer (NK), dendritic, or B cells). These cells may also be pluripotent stem cells (PSCs) such as human embryonic stem cells and induced PSCs, which can be used to generate therapeutic cell populations. In some embodiments. pluripotent and multipotent cells are differentiated into a desired cell type in vitro before being implanted into the patient.

In some alternatives, the genetically modified cell preparation is a T lymphocyte cell preparation. In some embodiments the T lymphocyte cell preparation comprises CD4+ T cells that have a chimeric receptor comprising an extracellular antibody variable domain specific for a ligand associated with the disease or disorder, a spacer region, a transmembrane domain, and an intracellular signaling domain of a T cell receptor and a recombinant cytokine receptor as described herein. In other alternatives, an adoptive cellular immunotherapy composition further comprises a chimeric receptor modified CD8+ cytotoxic T lymphocyte cell preparation that provides a cellular immune response, wherein the cytotoxic T lymphocyte cell preparation comprises CD8+ T cells that have a chimeric receptor comprising an extracellular single chain antibody specific for a ligand associated with the disease or disorder, a spacer region, a transmembrane domain, and an intracellular signaling domain of a T cell receptor and a recombinant cytokine receptor as described herein. In some alternatives, the chimeric receptor modified T cell population of the disclosure persists in vivo for at least about 3 days or longer. In some alternatives, each of these populations can be combined with one another or other cell types to provide a composition. In some alternatives, the host cells are Treg cells.

Regulatory T (Treg) cells are involved in the maintenance of immunological self-tolerance and in mitigating deleterious immune responses to both self and non-self (alio) antigens. Tregs comprise both natural and induced subtypes. Natural Tregs (nTregs) are cells which originate as a separate cell lineage during development. Peripheral or induced Tregs (iTregs) differentiate from conventional T cell. In some embodiments, CD4+ T cells that are not nTregs and not iTregs are engineered into Tregs using the methods and compositions of the disclosure. In some embodiments, the CD4+ T cells are used to make Treg cells expressing recombinant cytokine receptors using the methods and compositions of the disclosure.

In some embodiments, the cell expressing the recombinant cytokine receptor of the disclosure is not naturally occurring. In some embodiments, the cell is an immune cell. In some embodiments, the cell is a lymphocyte. In some embodiments, the cell is a T cell. In some embodiment, the cell is a regulatory T cell (Treg). In some embodiments, the Treg is compared to a control Treg that is not transduced with a recombinant cytokine receptor. In other embodiments, the Treg is compared to a control Treg that is transduced with an empty vector lacking a recombinant cytokine receptor.

Thus, provided herein are regulatory T cells (Tregs) comprising recombinant cytokine receptors comprising an IL-2Rβ polypeptide tethered at its N-terminus to an IL-2 cytokine, which is activated in the absence of exogenous IL-2. In some embodiments, the recombinant cytokine receptor is able to signal within the Treg in the absence of exogenous IL-2. In some embodiments, the Treg comprises a recombinant cytokine receptor comprising an IL-2 cytokine, a linker polypeptide, an IL-2Rβ extracellular domain, an IL-2Rβ transmembrane domain, and an IL-2Rβ intracellular domain. In some embodiments, the IL-2 receptor binds to an IL-2 cytokine that is linked to the IL-2Rβ polypeptide by a polypeptide linker sequence. In some embodiments, the recombinant cytokine receptor does not comprise a T cell receptor activation domain or a T cell costimulatory domain, optionally wherein the T cell receptor activation domain or T cell costimulatory domain is a CD28 signaling domain. Thus, in some embodiments, there is provided a the Treg comprising a recombinant cytokine receptor comprising from N to C terminus: (I) an IL-2 cytokine, (II) a polypeptide linker, (III) an IL-2 receptor extracellular domain, (IV) an IL-2 receptor transmembrane domain, and (V) an IL-2 receptor intracellular domain. In some embodiments, the Treg comprises a recombinant cytokine receptor comprising from N to C terminus: (I) an IL-2 cytokine, (II) a polypeptide linker, (III) an IL-2Rβ extracellular domain, (IV) a transmembrane domain, and (V) an IL-2Rβ intracellular domain. In some embodiments, the Treg comprises a recombinant cytokine receptor comprising from N to C terminus: (I) an IL-2 cytokine, (II) a polypeptide linker, and (III) an IL-2Rβ polypeptide. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the Treg comprises a recombinant cytokine receptor comprising from N to C terminus: (I) an IL-2 cytokine comprising an amino acid sequence selected from the group consisting of SEQ ID NOs:4-6 and 12, (II) a polypeptide linker, (III) an IL-2Rβ extracellular domain, (IV) a transmembrane domain, and (V) an IL-2Rβ intracellular domain. In some embodiments, the Treg comprises a recombinant cytokine receptor comprising from N to C terminus: (I) an IL-2 cytokine comprising an amino acid sequence selected from the group consisting of SEQ ID NOs:4-6 and 12, (II) a polypeptide linker, and (III) an IL-2Rβ polypeptide. In some embodiments, the Treg comprises a recombinant cytokine receptor comprising an amino acid sequence selected from the group consisting of SEQ ID NOs:1-3 and 11. In some embodiments, the Treg comprises a recombinant cytokine receptor comprising an amino acid sequence comprising at least about 80%, at least about 85%, at least about 90%, at least about 95%, at least about 96%, at least about 97%, at least about 98%, at least about 99% or about 100% sequence identity with the amino acid sequence selected from the group consisting of SEQ ID NOs:1-3 and 11. In some embodiments, the Treg comprises a recombinant cytokine receptor comprising an IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence selected from the group consisting of SEQ ID NOs:4-6 and 12. In some embodiments, the IL-2 cytokine is tethered to the IL-2 cytokine receptor extracellular domain by a polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the extracellular domain is the IL-2Rβ extracellular domain. In some embodiments, the extracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain comprises the IL-2Rβ transmembrane domain. In some embodiments, the transmembrane domain is the IL-2Rβ extracellular domain. In some embodiments, the transmembrane domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain comprises the IL-2Rβ intracellular domain. In some embodiments, the intracellular domain is the IL-2Rβ intracellular domain. In some embodiments, the intracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, the Treg comprises a recombinant cytokine receptor comprising from N to C terminus: (I) an IL-2 cytokine comprising an amino acid sequence selected from the group consisting of SEQ ID NOs:4-6 and 12, (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15. In some embodiments, the Treg comprises a recombinant cytokine receptor comprising from N to C terminus: (I) an IL-2 cytokine comprising an amino acid sequence selected from the group consisting of SEQ ID NOs:4-6 and 12, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7.

In some embodiments, there is provided a Treg comprising a recombinant cytokine receptor comprising a wild-type IL-2 cytokine that is linked to the IL-2Rβ polypeptide by a polypeptide linker sequence. Thus, in some embodiments, the Treg comprises a recombinant cytokine receptor comprising from N to C terminus: (I) a wild-type IL-2 cytokine; (II) a polypeptide linker, (III) an IL-2Rβ extracellular domain, (IV) a transmembrane domain, and (V) an IL-2Rβ intracellular domain. In some embodiments, the Treg comprises a recombinant cytokine receptor comprising from N to C terminus: (I) a wild-type IL-2 cytokine; (II) a polypeptide linker, (III) an IL-2Rβ extracellular domain, (IV) a transmembrane domain, and (V) an IL-2Rβ intracellular domain, wherein the recombinant cytokine receptor does not comprise a T cell receptor activation domain or a T cell costimulatory domain, optionally wherein the T cell receptor activation domain or T cell costimulatory domain is a CD28 signaling domain. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81 and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the Treg comprises a recombinant cytokine receptor comprising from N to C terminus: (I) an IL-2 cytokine comprising the amino acid sequence of SEQ ID NO:4, (II) a polypeptide linker, (III) an IL-2Rβ extracellular domain, (IV) a transmembrane domain, and (V) an IL-2Rβ intracellular domain. In some embodiments, the Treg comprises a recombinant cytokine receptor comprising from N to C terminus: (I) an IL-2 cytokine comprising the amino acid sequence of SEQ ID NO:4, (II) a polypeptide linker, (III) an IL-2Rβ extracellular domain, (IV) a transmembrane domain, and (V) an IL-2Rβ intracellular domain, wherein the recombinant cytokine receptor does not comprise a T cell receptor activation domain or a T cell costimulatory domain, optionally wherein the T cell receptor activation domain or T cell costimulatory domain is a CD28 signaling domain. In some embodiments, the Treg comprises a recombinant cytokine receptor comprising from N to C terminus: (I) an IL-2 cytokine comprising the amino acid sequence of SEQ ID NO:4, (II) a polypeptide linker, and (III) an IL-2Rβ polypeptide. In some embodiments, the Treg comprises a recombinant cytokine receptor comprising from N to C terminus: (I) an IL-2 cytokine comprising the amino acid sequence of SEQ ID NO:4, (II) a polypeptide linker, and (III) an IL-2Rβ polypeptide, wherein the recombinant cytokine receptor does not comprise a T cell receptor activation domain or a T cell costimulatory domain, optionally wherein the T cell receptor activation domain or T cell costimulatory domain is a CD28 signaling domain. In some embodiments, the Treg comprises a recombinant cytokine receptor comprising the amino acid sequence set forth in SEQ ID NO:1. In some embodiments, the Treg comprises a recombinant cytokine receptor comprising an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:1. In some embodiments, the Treg comprises a recombinant cytokine receptor comprising a wild-type IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:4. In some embodiments, the IL-2 cytokine is tethered to the IL-2 cytokine receptor extracellular domain by a polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the extracellular domain is the IL-2Rβ extracellular domain. In some embodiments, the extracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain comprises the IL-2Rβ transmembrane domain. In some embodiments, the transmembrane domain is the IL-2Rβ extracellular domain. In some embodiments, the transmembrane domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain comprises the IL-2Rβ intracellular domain. In some embodiments, the intracellular domain is the IL-2Rβ intracellular domain. In some embodiments, the intracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments the Treg comprises a recombinant cytokine receptor comprising from N to C terminus: (I) an IL-2 cytokine comprising the amino acid sequence of SEQ ID NO:4, (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15. In some embodiments the Treg comprises a recombinant cytokine receptor comprising from N to C terminus: (I) an IL-2 cytokine comprising the amino acid sequence of SEQ ID NO:4, (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15, wherein the recombinant cytokine receptor does not comprise a T cell receptor activation domain or a T cell costimulatory domain, optionally wherein the T cell receptor activation domain or T cell costimulatory domain is a CD28 signaling domain. In some embodiments, the Treg comprises a recombinant cytokine receptor comprising from N to C terminus: (I) an IL-2 cytokine comprising the amino acid sequence of SEQ ID NO:4, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7. In some embodiments, the Treg comprises a recombinant cytokine receptor comprising from N to C terminus: (I) an IL-2 cytokine comprising the amino acid sequence of SEQ ID NO:4, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7, wherein the recombinant cytokine receptor does not comprise a T cell receptor activation domain or a T cell costimulatory domain, optionally wherein the T cell receptor activation domain or T cell costimulatory domain is a CD28 signaling domain.

In some embodiments, there is provided a Treg comprising a recombinant cytokine receptor comprising an IL-2 cytokine comprising one or more amino acid substitutions. In some embodiments, the one or more substitutions decreases binding or affinity between i) the IL-2Rα cytokine receptor extracellular domain and the IL-2 cytokine and/or ii) the IL-2Rγ cytokine receptor extracellular domain and the IL-2 cytokine. In some embodiments, the one or more substitutions does not or only minimally decreases binding or affinity between the IL-2Rβ extracellular domain and the IL-2 cytokine. In some embodiments, the IL-2 cytokine comprises at least one or more amino acid substitution at positions selected from amino acid positions 42, 38, 61, 35, 18, 22, 126, and 43. In some embodiments, the IL-2 cytokine comprises at least one or more amino acid substitution at positions selected from amino acid positions 18, 22, 126, 38, 43 and 61. In some embodiments, the IL-2 cytokine is linked to the IL-2Rβ polypeptide by a polypeptide linker, wherein the amino acid-substituted IL-2 cytokine comprises one or more of the amino acid substitutions selected from the group consisting of R38D, K43E, and E61R. In some embodiments, the amino acid-substituted IL-2 cytokine may comprise additional amino acid substitutions. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the Treg comprises a recombinant cytokine receptor comprising an amino acid-substituted IL-2 cytokine that is linked to the IL-2Rβ polypeptide by a polypeptide linker sequence, wherein the mutated IL-2 cytokine comprises the amino acid substitutions R38D, K43E, and E61R. Thus, in some embodiments, the Treg comprises a recombinant cytokine receptor comprising from N to C terminus (I) a mutated IL-2 cytokine comprising the amino acid substitutions R38D, K43E, and E61R (“3×IL-2”), (II) a polypeptide linker, (III) an IL-2 receptor extracellular domain, (IV) a transmembrane domain, and (V) an IL-2 receptor intracellular domain. In some embodiments, the Treg comprises a recombinant cytokine receptor comprising from N to C terminus: (I) an IL-2 cytokine comprising the amino acid sequence of SEQ ID NO:5, (II) a polypeptide linker, (III) an IL-2Rβ extracellular domain, (IV) a transmembrane domain, and (V) an IL-2Rβ intracellular domain. In some embodiments, the Treg comprises a recombinant cytokine receptor comprising from N to C terminus: (I) an IL-2 cytokine comprising the amino acid sequence of SEQ ID NO:5, (II) a polypeptide linker, and (III) an IL-2Rβ polypeptide. In some embodiments, the Treg comprises a recombinant cytokine receptor comprising the amino acid sequence set forth in SEQ ID NO:2. In some embodiments, the Treg comprises a recombinant cytokine receptor comprising an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:2. In some embodiments the Treg comprises a recombinant cytokine receptor comprising a 3×-IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:5. In some embodiments, the IL-2 cytokine is tethered to the IL-2 cytokine extracellular domain by a polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the extracellular domain is the IL-2Rβ extracellular domain. In some embodiments, the extracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain comprises the IL-2Rβ transmembrane domain. In some embodiments, the transmembrane domain comprises the IL-2Rβ transmembrane domain. In some embodiments, the transmembrane domain is the IL-2Rβ extracellular domain. In some embodiments, the transmembrane domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain comprises the IL-2Rβ intracellular domain. In some embodiments, the intracellular domain comprises the IL-2Rβ intracellular domain. In some embodiments, the intracellular domain is the IL-2Rβ intracellular domain. In some embodiments, the intracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, the 3×IL-2 cytokine displays reduced or no binding to IL-2Rα and/or IL-2Rγ, but does not display reduced or eliminated binding to IL-2Rβ. In some embodiments, the Treg comprises a recombinant cytokine receptor comprising from N to C terminus: (I) an IL-2 cytokine comprising the amino acid sequence of SEQ ID NO:5, (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15. In some embodiments, the Treg comprises a recombinant cytokine receptor comprising from N to C terminus: (I) an IL-2 cytokine comprising the amino acid sequence of SEQ ID NO:5, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7.

In some embodiments, there is provided a Treg comprising a recombinant cytokine receptor comprising an IL-2 cytokine comprising one or more of the amino acid substitutions selected from the group consisting of L18R, Q22E, and Q126H. In some embodiments, the one or more substitutions decreases binding or affinity between i) the IL-2Rα cytokine receptor extracellular domain and the IL-2 cytokine and/or ii) the IL-2Rγ cytokine receptor extracellular domain and the IL-2 cytokine. In some embodiments, the one or more substitutions does not or only minimally decreases binding or affinity between the IL-2Rβ extracellular domain and the IL-2 cytokine. In some embodiments, the mutated IL-2 cytokine may comprise additional amino acid substitutions. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the Treg comprises a recombinant cytokine receptor comprising a mutated IL-2 cytokine that is linked to the IL-2Rβ polypeptide by a polypeptide linker sequence, wherein the mutated IL-2 cytokine comprises the amino acid substitutions L18R, Q22E, and Q126H. Thus, in some embodiments, the Treg comprises a recombinant cytokine receptor comprising from N to C terminus: (I) a mutated IL-2 cytokine comprising the amino acid substitutions L18R, Q22E, and Q126H (“REH IL-2”), (II) a polypeptide linker, (III) an IL-2 receptor extracellular domain, (IV) a transmembrane domain, and (V) an IL-2 receptor intracellular domain. In some embodiments, the Treg comprises a recombinant cytokine receptor comprising from N to C terminus: (I) an IL-2 cytokine comprising the amino acid sequence of SEQ ID NO:6, (II) a polypeptide linker, (III) an IL-2Rβ extracellular domain, (IV) a transmembrane domain, and (V) an IL-2Rβ intracellular domain. In some embodiments, the Treg comprises a recombinant cytokine receptor comprising from N to C terminus: (I) an IL-2 cytokine comprising the amino acid sequence of SEQ ID NO:6, (II) a polypeptide linker, and (III) an IL-2Rβ polypeptide. In some embodiments, the Treg comprises a recombinant cytokine receptor comprising the amino acid sequence set forth in SEQ ID NO:3. In some embodiments, the Treg comprises a recombinant cytokine receptor comprising an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:3. In some embodiments, the Treg comprises a recombinant cytokine receptor comprising an REH IL-2 molecule having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:6. In some embodiments, the IL-2 cytokine is tethered to the IL-2 cytokine extracellular domain by a polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the extracellular domain is the IL-2Rβ extracellular domain. In some embodiments, the extracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain comprises the IL-2Rβ transmembrane domain. In some embodiments, the transmembrane domain comprises the IL-2Rβ transmembrane domain. In some embodiments, the transmembrane domain is the IL-2Rβ extracellular domain. In some embodiments, the transmembrane domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain comprises the IL-2Rβ intracellular domain. In some embodiments, the intracellular domain comprises the IL-2Rβ intracellular domain. In some embodiments, the intracellular domain is the IL-2Rβ intracellular domain. In some embodiments, the intracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, the REH IL-2 cytokine displays reduced or no binding to IL-2Rα and/or IL-2Rγ, but does not display reduced or eliminated binding to IL-2Rβ. In some embodiments, the Treg comprises a recombinant cytokine receptor comprising from N to C terminus: (I) an IL-2 cytokine comprising the amino acid sequence of SEQ ID NO:6, (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15. In some embodiments, the Treg comprises a recombinant cytokine receptor comprising from N to C terminus: (I) an IL-2 cytokine comprising the amino acid sequence of SEQ ID NO:6, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7.

In some embodiments, there is provided a Treg comprising a recombinant cytokine receptor comprising an IL-2 cytokine comprising one or more of the amino acid substitutions selected from the group consisting of L18R, Q22E, Q126H, R38D, K43E, and E61R. In some embodiments, the IL-2 cytokine may comprise additional amino acid substitutions. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the Treg comprises a recombinant cytokine receptor comprising a mutated IL-2 cytokine that is linked to the IL-2Rβ polypeptide by a polypeptide linker sequence, wherein the mutated IL-2 cytokine comprises the amino acid substitutions L18R, Q22E, Q126H, R38D, K43E, and E61R. In some embodiments, the one or more substitutions decreases binding or affinity between i) the IL-2Rα cytokine receptor extracellular domain and the IL-2 cytokine and/or ii) the IL-2Rγ cytokine receptor extracellular domain and the IL-2 cytokine. In some embodiments, the one or more substitutions does not or only minimally decreases binding or affinity between the IL-2Rβ extracellular domain and the IL-2 cytokine. Thus, in some embodiments, the Treg comprises a recombinant cytokine receptor comprising from N to C terminus: (I) a mutated IL-2 cytokine comprising the amino acid substitutions L18R, Q22E, Q126H, R38D, K43E, and E61R (“3×REH IL-2”), (II) a polypeptide linker, (III) an IL-2 receptor extracellular domain, (IV) a transmembrane domain, and (V) an IL-2 receptor intracellular domain. In some embodiments, the Treg comprises a recombinant cytokine receptor comprising from N to C terminus: (I) an IL-2 cytokine comprising the amino acid sequence of SEQ ID NO:12, (II) a polypeptide linker, (III) an IL-2Rβ extracellular domain, (IV) a transmembrane domain, and (V) an IL-2Rβ intracellular domain. In some embodiments, there is provided a recombinant cytokine receptor comprising from N to C terminus: (I) an IL-2 cytokine comprising the amino acid sequence of SEQ ID NO:12, (II) a polypeptide linker, and (III) an IL-2Rβ polypeptide. In some embodiments, the Treg comprises a recombinant cytokine receptor comprising the amino acid sequence set forth in SEQ ID NO:11. In some embodiments, the Treg comprises a recombinant cytokine receptor comprising an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:11. In some embodiments, the Treg comprises a recombinant cytokine receptor comprising a 3×REH IL-2 cytokine having an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:12. In some embodiments, the IL-2 cytokine is tethered to the IL-2 cytokine receptor extracellular domain by a polypeptide linker. In some embodiments, the polypeptide linker comprises glycines and serines. In some embodiments, the polypeptide linker comprises an amino acid sequence selected from the group consisting of SEQ ID NOs:9 and 16-21. In some embodiments, the extracellular domain comprises the IL-2Rβ extracellular domain. In some embodiments, the extracellular domain is the IL-2Rβ extracellular domain. In some embodiments, the extracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:13. In some embodiments, the transmembrane domain comprises the IL-2Rβ transmembrane domain. In some embodiments, the transmembrane domain comprises the IL-2Rβ transmembrane domain. In some embodiments, the transmembrane domain is the IL-2Rβ extracellular domain. In some embodiments, the transmembrane domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:14. In some embodiments, the intracellular domain comprises the IL-2Rβ intracellular domain. In some embodiments, the intracellular domain comprises the IL-2Rβ intracellular domain. In some embodiments, the intracellular domain is the IL-2Rβ intracellular domain. In some embodiments, the intracellular domain comprises an amino acid sequence comprising at least about 80%, at least about 85%, at least about 90%, at least about 95%, at least about 96%, at least about 97%, at least about 98%, at least about 99% or about 100% sequence identity with the amino acid sequence set forth in SEQ ID NO:15. In some embodiments, the 3×REH IL-2 cytokine displays reduced or no binding to IL-2Rα and/or IL-2R7, but does not display reduced or eliminated binding to IL-2Rβ. In some embodiments, the Treg comprises a recombinant cytokine receptor comprising from N to C terminus: (I) an IL-2 cytokine comprising the amino acid sequence of SEQ ID NO:12, (II) a polypeptide linker comprising an amino acid sequence selected from the group consisting of SEQ ID NOs:9 and 16-21, (III) an IL-2Rβ extracellular domain comprising the amino acid sequence of SEQ ID NO:13, (IV) an IL-2Rβ transmembrane domain comprising the amino acid sequence of SEQ ID NO:14, and (V) an IL-2Rβ intracellular domain comprising the amino acid sequence of SEQ ID NO:15. In some embodiments, the Treg comprises a recombinant cytokine receptor comprising from N to C terminus: (I) an IL-2 cytokine comprising the amino acid sequence of SEQ ID NO:12, (II) a polypeptide linker comprising an amino acid sequence selected from the group consisting of SEQ ID NOs:9 and 16-21, and (III) an IL-2Rβ polypeptide comprising the amino acid sequence of SEQ ID NO:7.

In some embodiments, the Treg expressing the recombinant cytokine receptor of the disclosure is not naturally occurring (not an nTreg and/or not an iTreg). In some embodiments, the cell expresses one or more markers characteristic of a Treg. Treg markers include high levels of IL-2Rα (IL-2Rα+), low levels of CD127 (CD127lo), or both high IL-2Rβ and low CD127. The levels of IL-2Rα and CD127 are compared, for example to a CD4+ T cell that is not a Treg. In some embodiments, the Treg cell expressing the recombinant cytokine receptor has a high IL-2Rα, high CD4, and low CD127 phenotype.

Also provided herein is a cell that expresses a recombinant cytokine receptor. In some embodiments, the recombinant cytokine receptor comprises an IL-2 receptor beta chain polypeptide, a linker polypeptide, and a human IL-2 cytokine. In some embodiments, the recombinant cytokine receptor comprises a human IL-2 cytokine selected from the group consisting of WT IL-2, 3×IL-2, REH IL-2, and 3×REH IL-2. In some embodiments, the recombinant cytokine receptor does not comprise a T cell receptor activation domain or a T cell costimulatory domain, optionally wherein the T cell receptor activation domain or T cell costimulatory domain is a CD28 signaling domain.

In some embodiments, the cell expresses one or more proteins associated with a Treg phenotype. In some embodiments, the cell expresses FOXP3. FOXP3 plays a crucial role in development and function of Treg cells (Yagi et al., “Crucial role of FOXP3 in the development and function of human IL-2Rα+CD4+ regulatory T cells,” int Immunol. 2004 November; 16(11):1643-56. Epub 2004 Oct. 4; Sadlon et al.) “Unravelling the molecular basis for regulatory T-cell plasticity and loss of function in disease,” Clinical & Translational Immunology, 2018). FOXP3 is initially expressed during the expansion of T cells for a first time but is followed by the loss of FOXP3 expression after polyclonal stimulation. This is in contrast to Tregs, where FOXP3 expression rise and are maintained over time. Expression levels of FOXP3 may be assessed by conventional methods such as Western blotting, flow cytometry or ELISA. Expression levels may also be assessed by analyzing mRNA using techniques such as RT-qPCR. In some embodiments, expression of FOXP3 increases compared to untransduced cells. In some embodiments, expression of FOXP3 increases compared to cells without recombinant cytokine receptors.

In some embodiments, FOXP3 expression increases at least 2-fold, at least 3-fold, at least 4-fold, at least 5-fold compared to cells without recombinant cytokine receptors. In some embodiments, FOXP3 expression increases at least 1.1-fold, at least 1.5-fold, 2-fold, at least 3-fold, at least 4-fold, at least 5-fold compared to cells that have not been transduced with a recombinant cytokine receptor. In some embodiments, the expression of FOXP3 is increased in the Tregs transduced with a recombinant cytokine receptor compared to untransduced T cells cultured without one or more cytokines. In some embodiments, FOXP3 expression is maintained at approximately the same level as the day of highest FOXP3 expression, following transduction with the recombinant cytokine receptor.

In some embodiments, the Treg cell is CD4 positive (CD4+). In some embodiments, the cell is CD4+/IL-2Rα+. In some embodiments, the cell is CD4+/CD127 low. In some embodiments, the cell is CD4+/IL-2Rα+/CD127 low.

Tregs can be characterized by expression of IL-2Rα+. In some embodiments, the cell is IL-2Rα+. In some embodiments, the cell is CD4+/IL-2Rα+. In some embodiments, the cell is IL-2Rα+/CD127 low. In some embodiments, CD4+/IL-2Rα+/CD127 low.

In some embodiments, the cell expresses a low level of CD127 (CD127lo). In some embodiments, the cell is CD4+/CD127 low. In some embodiments, the cell is IL-2Rα+/CD127 low. In some embodiments. CD4+/IL-2Rα+/CD127 low. In some embodiments, cells transduced with the recombinant cytokine receptors provided herein maintain CD127 low status upon transduction.

HELIOS is a transcription factor that is expressed in Tregs. In some embodiments, the Treg cells of the present disclosure expresses HELIOS. In some embodiments, the expression of HELIOS is increased compared to non-Treg T cells. In some embodiments, the expression of HELIOS is detectable compared to non-Treg T cells. Expression levels of HELIOS may be assessed by conventional methods such as Western blotting. flow cytometry or ELISA. Expression levels may also be assessed by analyzing mRNA using techniques such as RT-PCR. In some embodiments, HELIOS expression increases at least 2-fold, at least 3-fold, at least 4-fold, at least 5-fold. at least 6-fold, at least 7-fold, at least 8-fold, at least 9-fold, at least 10-fold, or at least 20-fold compared to non-Treg T cells. In some embodiments, the expression of HELIOS is increased in the Tregs transduced with recombinant cytokine receptor compared to cells cultured without cytokine. In some embodiments, the expression of HELIOS is increased in the Tregs transduced with a recombinant cytokine receptor compared to untransduced. In some embodiments, Tregs transduced with a recombinant cytokine receptor maintain HELIOS expression following transduction.

In some embodiments, the relative amount of Treg cells in a composition comprising a population of Treg cells transduced with the recombinant cytokine receptor increases over time. In some embodiments, the population of Treg cells transduced with the recombinant cytokine receptor grown without IL-2 contains a similar number of viable cells compared to a composition comprising a population of the same Treg cells not transduced with the recombinant cytokine receptor grown with IL-2. In some embodiments, at least 80% of the population of Treg cells transduced with the recombinant cytokine maintain FOXP3 and/or HELIOS expression 14 days after transduction. In some embodiments, at least 80% of the cells in the population of Treg cells transduced with the recombinant cytokine maintain FOXP3 and/or HELIOS expression 23 days after transduction.

In some embodiments, the population of Treg cells transduced with the recombinant cytokine receptor expands at least two-fold more than a population of the same Treg cells not transduced with the recombinant cytokine receptor. In some embodiments, the population of Treg cells transduced with the recombinant cytokine receptor expands at least two-fold, and wherein the population of Treg cells transduced with the recombinant cytokine receptor maintains expression of least one Treg marker selected from the group consisting of CD25+, FOXP3, and HELIOS.

In some embodiments, the relative amount of Treg cells in a composition comprising a population of Treg cells transduced with the recombinant cytokine receptor increases over time when cultured without IL-2.

In some embodiments, the cell has an immunosuppressive phenotype. In one embodiment, the cell generates an immunosuppressive effect in an individual having an immune-related disorder. In some embodiments, the individual is human. In some embodiments, the cell is autologous to the individual.

In some embodiment, the cell suppresses, blocks, or inhibits Graft-vs-host disease (GvHD) in the individual. In one embodiment, the cell suppresses, blocks, or inhibits an immune-related disorder in the individual. In some embodiments, the cell is administered prior to onset of an immune-related disorder. In some embodiments, the individual is human. In some embodiments, the cell is autologous to the individual. In some embodiments, the cell is allogeneic to the individual.

Methods of measuring markers used to characterize Treg cells will be readily apparent to the person of ordinary skill in the art. For example, Tregs or populations of T cells comprising Tregs can be cultured using the methods described herein. Following culturing, Tregs can be collected and stained using antibodies against Treg markers such as FOXP3 (PE), IL-2Rα (APC), and CD127 (BV421) and expression analyzed using fluorescence activated flow cytometry (FACS) or fluorescence microscopy. Gene expression may be measured by methods such as RT-qPCR.

### III. Methods of Preparation

The recombinant cytokine receptors described herein may be prepared and transduced into host cells, e.g., Treg cells. Sequences encoding the recombinant cytokine receptors can be synthesized. After obtaining such sequence, it is cloned into a suitable expression vector, then transduced into a suitable host cell. The transduced host cells are recovered and cultured to obtain viable host cells that stably express the recombinant cytokine receptors of the present invention.

In some embodiments, the present application provides isolated nucleic acids encoding one or more of the polypeptides of any one of the recombinant cytokine receptors. The isolated nucleic acids may be DNA. In some embodiments, the isolated nucleic acid is inserted into a vector, such as an expression vector, a viral vector, or a cloning vector. For expression of the nucleic acids, the vector may be introduced into a host cell to allow expression of the nucleic acids within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art. For example, the promoter sequences may be selected to promote the transcription of the polynucleotide in the vector. Suitable promoter sequences include, without limitation, MND, 17 promoter, T3 promoter, SP6 promoter, beta-actin promoter, EF1a promoter, CMV promoter, and SV40 promoter. Enhancer sequences may be selected to enhance the transcription of the nucleic acids. Selectable markers may be selected to allow selection of the host cells inserted with the vector from those not, for example, a selectable marker may be a transmembrane gene such as EGFR that can be identified by flow cytometry and FACS analysis. In some embodiments, the signal sequence comprises the amino acid sequence of SEQ ID NO:8.

In some embodiments, the nucleic acid (e.g., vector, such as an expression vector, a viral vector, or a cloning vector) expresses an antigen receptor and/or another additional polypeptide. The antigen receptor may be, for example, an antibody, an engineered antibody such as an scFv, a CAR, an engineered TCR, a TCR mimic or a chimeric antibody-T cell receptor, or a chimeric signaling receptor. The antigen receptor may target an antigen of interest (e.g., a tumor antigen or an antigen of a pathogen or a target in an inflammation site). The antigens may include, without limitation, AFP (alpha-fetoprotein), αvβ6 or another integrin, BCMA, B7-H3, B7-H6, CA9 (carbonic anhydrase 9), CCL-1 (C—C motif chemokine ligand 1), CD5, CD19, CD20. CD21, CD22, CD23, CD24, CD30, CD33, CD38, CD40, CD44, CD44v6, CD44v7/8, CD45, CD47, CD56, CD66e, CD70. CD74, CD79a, CD79b, CD98, CD123, CD138, CD171, CD352, CEA (carcinoembryonic antigen), Claudin, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor). FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens.

In some embodiments, the construct comprising the recombinant cytokine receptors described herein comprises a cleavable linker. In some embodiments, the cleavable linker is a 2A polypeptide. In some embodiments, the construct comprising the recombinant cytokine receptor comprises a nucleotide sequence encoding a P2A linker between a nucleotide sequence encoding the recombinant cytokine receptor and a marker (such as EGFR). In some embodiments, 2A-like sequences, or “peptide bond-skipping” 2A sequences, are derived from, for example, many different viruses, including, for example, from Thosea asigna. These sequences are sometimes also known as “peptide skipping sequences.” When this type of sequence is placed within a cistron between two polypeptides that are intended to be separated, the ribosome appears to skip a peptide bond; in the case of the Thosea asigna sequence, the bond between the Gly and Pro amino acids at the carboxy terminal “P-G-P” is omitted. This may leave two to three polypeptides, for example, an inducible chimeric pro-apoptotic polypeptide and a chimeric antigen receptor, or, for example, a marker polypeptide and an inducible chimeric pro-apoptotic polypeptide. When this sequence is used, the polypeptide that is encoded 5′ of the 2A sequence may end up with additional amino acids at the carboxy terminus, including the Gly residue and any upstream residues in the 2A sequence. The peptide that is encoded 3′ of the 2A sequence may end up with additional amino acids at the amino terminus, including the Pro residue and any downstream residues following the 2A sequence. In some embodiments, the cleavable linker is a 2A polypeptide derived from porcine teschovirus-1 (P2A). In some embodiments, the 2A cotranslational sequence is a 2A-like sequence. In some embodiments, the 2A cotranslational sequence is T2A (Thosea asigna virus 2A), F2A (foot and mouth disease virus 2A), P2A (porcine teschovirus-1 2A), BmCPV 2A (cytoplasmic polyhedrosis virus 2A), BmIFV 2A (flacherie virus of B. mori 2A), or E2A (equine rhinitis A virus 2A). In some embodiments, the 2A cotranslational sequence is T2A-GSG, F2A-GSG, P2A-GSG, or E2A-GSG. In some embodiments, the 2A cotranslational sequence is selected from the group consisting of T2A, P2A, and F2A. By “cleavable linker” is meant that the linker is cleaved by any means, including, for example, nonenzymatic means, such as peptide skipping, or enzymatic means (see, e.g., Donnelly, ML (2001), J. Gen. Virol. 82:1013-25, hereby incorporated by reference in its entirety). In a specific embodiment, a P2A comprises (or consists of) a sequence disclosed herein. In a specific embodiment, a P2A comprises (or consists of) a sequence disclosed herein (e.g., a sequence disclosed in the Examples below).

In certain embodiments, a 2A linker includes the amino acid sequence of SEQ ID NO:10 (SGATNFSLLKQAGDVEENPGP). In certain embodiments, the 2A linker further includes a GSG amino acid sequence at the amino terminus of the polypeptide; in other embodiments, the 2A linker includes a GSGPR amino acid sequence (SEQ ID NO: 21) at the amino terminus of the polypeptide. Thus, by a “2A” sequence, the term may refer to a 2A sequence in an example described herein or may also refer to a 2A sequence as listed herein further comprising a GSG or GSGPR sequence (SEQ ID NO: 21) at the amino terminus of the linker.

In some embodiments, the host cell (e.g., Treg cell) contains the vector described above. The vector can be introduced to the cell using any suitable methods known in the art, including, but not limited to, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transduction of cells for expression of a vector of interest are well known in the art. In some embodiments, the host cells comprise a vector comprising the isolated nucleic acid encoding the recombinant cytokine receptor.

In some embodiments, the present application provides methods of expressing any of the recombinant cytokine receptors described herein, comprising culturing the isolated host cell containing the vector and recovering the recombinant cytokine receptor from the cell culture. The isolated host cells are cultured under conditions that allow expression of the isolated nucleic acids inserted in the vectors. Suitable conditions for expression of polynucleotides may include, without limitation, suitable medium, suitable density of host cells in the culture medium. presence of necessary nutrients, presence of supplemental factors, suitable temperatures and humidity, and absence of microorganism contaminants. A person with ordinary skill in the art can select the suitable conditions as appropriate for the purpose of the expression.

### Vectors

In certain aspects. the present disclosure provides nucleic acid molecules that encode any one or more of the recombinant cytokine receptors described herein. Such nucleic acid molecules can be inserted into an appropriate vector (e.g., viral vector or non-viral plasmid vector) for introduction in a host regulatory T cell (Treg) of interest.

As used herein, the term “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications introducing expressible nucleic acid molecules encoding proteins, fusion proteins or enzymes, or other nucleic acid molecule additions, deletions. substitutions, or other functional disruption of a cell's genetic material. Additional modifications include, for example, non-coding regulatory regions in which the modifications alter expression of a gene or operon. In some embodiments, a cell, such as a regulatory T cell (Treg), obtained from a subject may be converted into a non-natural or recombinant regulatory T cell (Treg) (e.g., a non-natural or recombinant regulatory T cell (Treg)) by introducing a nucleic acid that encodes a recombinant cytokine receptor as described herein and whereby the cell expresses a cell surface located recombinant cytokine receptor.

A vector that encodes a core virus is referred to herein as a “viral vector.” There are a large number of available viral vectors suitable for use with the compositions of the instant disclosure, including those identified for human gene therapy applications (see Pfeifer and Verma (2001), Ann. Rev. Genomics Hum. Genet. 2:177, hereby incorporated by reference in its entirety). Suitable viral vectors include vectors based on RNA viruses, such as retrovirus-derived vectors, e.g., Maloney murine leukemia virus (MLV)-derived vectors, and include more complex retrovirus-derived vectors, e.g., lentivirus-derived vectors. HIV-1-derived vectors belong to this category. Other examples include lentivirus vectors derived from HIV-2. FIV, equine infectious anemia virus, SIV, and Maedi-Visna virus (ovine lentivirus). Methods of using retroviral and lentiviral viral vectors and packaging cells for transducing mammalian host regulatory T cells (Treg) with viral particles containing chimeric antigen receptor transgenes are known in the art and have been previously described, for example, in U.S. Pat. No. 8,119,772; Walchli et al. (2011), PLoS One 6:327930; Zhao et al. (2005), J. Immunol. 174:4415; Engels et al. (2003), Hum. Gene Ther. 14:1155; Frecha et al. (2010), Mol. Ther. 18:1748; and Verhoeyen et al. (2009), Methods Mol. Biol. 506:97. Retroviral and lentiviral vector constructs and expression systems are also commercially available.

In some embodiments, a viral vector is used to introduce a non-endogenous nucleic acid sequence encoding a recombinant cytokine receptor. A viral vector may be a retroviral vector or a lentiviral vector. A viral vector may also include nucleic acid sequences encoding a marker for transduction. Transduction markers for viral vectors are known in the art and include selection markers, which may confer drug resistance, or detectable markers, such as fluorescent markers or cell surface proteins that can be detected by methods such as flow cytometry. In particular embodiments, a viral vector further comprises a gene marker for transduction comprising green fluorescent protein, an extracellular domain of human CD2, or a truncated human EGFR (huEGFRt; see Wang et al. (2011), Blood 118:1255). When a viral vector genome comprises a plurality of nucleic acid sequences to be expressed in a host cell (e.g., T cell such as Treg) as separate transcripts, the viral vector may also comprise additional sequences between the two (or more) transcripts allowing bicistronic or multicistronic expression. Examples of such sequences used in viral vectors include internal ribosome entry sites (IRES), furin cleavage sites, viral 2A peptide, or any combination thereof.

Other vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al. (1998), Gene Ther. 5: 1517).

Other vectors recently developed for gene therapy uses can also be used with the compositions and methods of this disclosure. Such vectors include those derived from baculoviruses and α-viruses. (Jolly, DJ (1999), Emerging Viral Vectors, pp 209-40 in Friedmann T. ed. The Development of Human Gene Therapy. New York: Cold Spring Harbor Lab), or plasmid vectors (such as sleeping beauty or other transposon vectors).

In certain embodiments, hematopoietic progenitor cells or embryonic stem cells are modified to comprise a non-endogenous nucleic acid molecule that encodes a recombinant cytokine receptor of this disclosure. Hematopoietic progenitor cells may comprise induced pluripotent stem cells, which may be derived or originate from fetal liver tissue, bone marrow, cord blood, or peripheral blood. The hematopoietic progenitor cells may be from human, mouse, rat, or other mammals.

In certain embodiments, the host cell transfected to express a recombinant cytokine receptor of this disclosure is a functional regulatory T cell (Treg). One or more growth factor cytokines that promote proliferation of regulatory T cells (Treg) expressing a recombinant cytokine receptor of this disclosure may be added to the culture. The cytokines may be human or non-human. Exemplary growth factor cytokines that may be used promote regulatory T cell (Treg) proliferation include IL-2, TGFβ, or similar.

### IV. Methods of Expanding and Culturing Cells

In some embodiments, the cell transfected to express a recombinant cytokine receptor of this disclosure is a eukaryotic cell. In some embodiments, the cell transfected to express a recombinant cytokine receptor of this disclosure is a human cell. In certain embodiments, the cell transfected to express a recombinant cytokine receptor of this disclosure is an immune cell. In certain embodiments, the cell transfected to express a recombinant cytokine receptor of this disclosure is a T cell. In certain embodiments, the cell transfected to express a recombinant cytokine receptor of this disclosure is a functional regulatory T cell (Treg).

In some embodiments the Treg cell is isolated from peripheral blood mononuclear (PBMC) cells. In some embodiments, the Treg cell is isolated from PBMCs using density gradient centrifugation. In some embodiments, the Treg cells are enriched by positive selection. In some embodiments, the Treg cells are enriched by positive selection for IL-2Rα+. In some embodiments, the Treg cells are enriched by positive selection for CD4+IL-2Rα+CD127lo cells. In some embodiments, the enrichment occurs by FACS. In some embodiments, the Tregs are stimulated by anti-CD3 and/or anti-CD28 on day 0 after positive selection. In some embodiments, the Tregs are re-stimulated by anti-CD3 and/or anti-CD28 on day 9 of culture after positive selection.

One or more growth factor cytokines may be added to the culture that promote proliferation of T cells such as Tregs, including Tregs that express recombinant cytokine receptors of this disclosure. The cytokines may be human or non-human cytokines. Exemplary growth factor cytokines that may be used to promote regulatory T cell (Treg) proliferation include IL-4, IL-7, IL-9, IL-21 or the like. In some embodiments, the cytokines are added to the media prior to selection. In some embodiments, the cytokines are added to the media after cell isolation. In some embodiments, cytokines are added to the cell culture approximately every 12 to 60, such as 24 to 48 hours. In some embodiments, the cytokines are added to the media for the duration of the culture.

In some embodiments, cells expressing the recombinant cytokine receptor of this disclosure are cultured for a sufficient time to induce proliferation or differentiation. The cells are maintained in culture generally for about 3 days to about 5 days, about 4 to about 10 days, about 5 to about 20 days, about 10 to about 23 days, about 15 to about 30 days, or about 23 to about 30 days. It will be appreciated that the cells may be maintained for an appropriate amount of time required to achieve a desired result, i.e., a desired cellular composition or level of proliferation. For example, to generate a cellular composition comprising primarily Tregs, cells may be maintained in culture for about 30 days.

In some embodiments, the method further comprises detecting one or more Treg markers provided herein. In some embodiments, the method further comprises detecting IL-2 signaling. In some embodiments, the method further comprises detecting phosphorylated STAT-5.

In some embodiments, the suppressive activity of the transduced Treg is increased upon transduction with a recombinant cytokine receptor provided herein. In some embodiments, the rate of division of a cytotoxic T cell is decreased by the transduced Treg cells provided herein. In some embodiments, the rate of division of a CD4+ T cell is decreased. In some embodiments, the rate of division of a CD8+ T cell is decreased. In some embodiments, the activity of a cytotoxic T cell is decreased.

In some embodiments, provided herein is a method of expanding a transduced Treg cell in the absence of exogenous IL-2 comprising culturing a cell expressing a recombinant cytokine receptor provided herein. In some embodiments, the cultured cell expresses a recombinant cytokine receptor wherein IL-2Rβ is tethered to an IL-2 cytokine. In some embodiments, the cultured cell comprising a recombinant cytokine receptor provided herein receives IL-2 signaling stimulus from the tethered IL-2 cytokine. In some embodiments, the cell is cultured in a culture medium comprising a cytokine other than IL-2. In some embodiments, the cell is cultured in a medium comprising two, three, four, or five or more cytokines.

### V. Pharmaceutical Compositions, Articles of Manufacture, and Kits

Further provided by the present application are pharmaceutical compositions comprising a cell, e.g., a T cell such as a Treg cell, comprising a recombinant cytokine receptor described herein.

The pharmaceutical compositions may be suitable for a variety of modes of administration described herein, including for example systemic or localized administration. In some embodiments, the pharmaceutical composition is formulated for intravenous administration.

The pharmaceutical compositions to be used for in vivo administration are generally formulated as sterile, substantially isotonic, and in full compliance with all Good Manufacturing Practice (GMP) regulations of the U.S. Food and Drug Administration. Sterility is readily accomplished by filtration through sterile filtration membranes. In some embodiments, the composition is free of pathogens. For injection, the pharmaceutical composition can be in the form of liquid solutions, for example in physiologically compatible buffers such as Hank's solution or Ringer's solution.

In some embodiments, the pharmaceutical composition is suitable for administration to a human. In some embodiments, the pharmaceutical composition is suitable for administration to a rodent (e.g., mice, rats) or non-human primates (e.g., Cynomolgus monkey). In some embodiments, the pharmaceutical composition is cryopreserved.

The present application also provides kits comprising compositions (such as pharmaceutical compositions) described herein and may further comprise instruction(s) on methods of using the composition, such as uses described herein. The kits described herein may further include other materials desirable from a commercial and user standpoint, including other buffers, diluents, filters, needles, syringes, and package inserts with instructions for performing any methods described herein.

### VI. Methods of Treatment

In some embodiments, provided herein is a method of treating an immune-related disorder comprising administering a cell (e.g., a Treg cell) comprising a recombinant cytokine receptor provided herein. In some embodiments, the recombinant cytokine receptor comprises an Interleukin-2 Receptor beta (IL-2Rβ or IL-2RB) polypeptide, wherein the N-terminus is tethered to an IL-2 cytokine. In some embodiments, the recombinant cytokine receptor used in the method of treating an immune-related disorder is able to signal in the absence of exogenous IL-2. In some embodiments, the recombinant cytokine receptor used in the method of treating an immune-related disorder comprises an IL-2 cytokine, a polypeptide linker, and an IL-2Rβ polypeptide. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. Thus, in some embodiments, there is provided a method of treating an immune-related disorder comprising administering a cell (e.g., a Treg cell) comprising a recombinant cytokine receptor comprising: (I) an IL-2 cytokine; (II) a polypeptide linker, and (III) an IL-2Rβ polypeptide. In some embodiments, the recombinant cytokine receptor comprises the amino acid sequence selected from the group consisting of SEQ ID NOs:1-3 and 11. In some embodiments, the recombinant cytokine receptor comprises an amino acid sequence comprising at least 80%, at least 85%, at least 90%, at least 95%, at least 96%, at least 97%, at least 98%, at least 99% or 100% sequence identity with the amino acid sequence selected from the group consisting of SEQ ID NOs:1-3 and 11. In some embodiments, the cell is an immune cell. In some embodiments, the cell is a lymphocyte. In some embodiments, the cell is a T cell. In some embodiment, the cell is a Treg.

In some embodiments, the method of treating an immune-related disorder comprises administering a cell (e.g., a Treg cell) comprising a recombinant cytokine receptor comprising IL-2Rβ tethered to a wild-type IL-2 cytokine. In some embodiments, the method comprises transducing the cell (e.g., the Treg cell) with a vector encoding the recombinant cytokine receptor. In some embodiments, the cells are autologous to the individual to be treated. In some embodiments, the wild-type IL-2 cytokine comprises the amino acid sequence set forth in SEQ ID NO:4. In some embodiments, the wild-type IL-2 cytokine comprises an amino acid sequence comprising at least 80%, at least 85%, at least 90%, at least 95%, at least 96%, at least 97%, at least 98%, at least 99% or 100% to SEQ ID NO:4. In some embodiments, the method of treating an immune disorder comprises transducing a cell (e.g., a Treg cell) with a recombinant cytokine receptor comprising in N-terminal to C-terminal direction: (I) a wild-type IL-2 cytokine; (IT) a polypeptide linker; and (III) an IL-2Rβ. In some embodiments, the recombinant cytokine receptor does not comprise a TCR activation domain or a costimulatory domain (e.g., a CD3 or a CD28 activation or costimulatory domain, such as a CD28 signaling domain). In some embodiments, the recombinant cytokine receptor comprises an amino acid sequence comprising at least 80%, at least 85%, at least 90%, at least 95%, at least 96%, at least 97%, at least 98%, at least 99% or 100% sequence identity with the amino acid sequence set forth in SEQ ID NO:1. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the cell is an immune cell. In some embodiments, the cell is a lymphocyte. In some embodiments, the cell is a T cell. In some embodiment, the cell is a Treg.

In some embodiments, the method of treating an immune-related disorder comprises administering a cell (e.g., a Treg cell) comprising a recombinant cytokine receptor comprising an IL-2 cytokine wherein the IL-2 cytokine comprises at one or more amino acid substitution at positions selected from amino acid positions 18, 22, 126, 38, 43, and 61. In some embodiments, the method of an immune-related disorder comprises administering a cell (e.g., a Treg cell) comprising a recombinant cytokine receptor comprising an IL-2 cytokine wherein the IL-2 cytokine comprises one or more amino acid substitutions selected from the group consisting of R38D, K43E, and E61R. In some embodiments, the method of treating an immune-related disorder comprises administering a cell (e.g., a Treg cell) comprising a recombinant cytokine receptor comprising an IL-2 cytokine wherein the IL-2 cytokine comprises amino acid substitutions R38D, K43E, and E61R (“3×IL-2”). In some embodiments, the method of treating an immune-related disorder comprises administering a cell (e.g., a Treg cell) comprising a recombinant cytokine receptor comprising an IL-2 cytokine wherein the IL-2 cytokine comprises amino acid substitutions R38D, K43E, and E61R and one or more additional amino acid substitutions. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the method comprises transducing the cell (e.g., Treg cell) with a vector encoding the recombinant cytokine receptor. In some embodiments, the cells are autologous to the individual to be treated. In some embodiments, the 3×IL-2 cytokine comprises the amino acid sequence set forth in SEQ ID NO:5. In some embodiments, the 3×IL-2 cytokine comprises an amino acid sequence comprising at least 80%, at least 85%, at least 90%, at least 95%, at least 96%, at least 97%, at least 98%, at least 99% or 100% to SEQ ID NO:5. In some embodiments, the method of treating an immune-related disorder comprising administering a cell (e.g., a Treg cell) comprising recombinant cytokine receptor comprises in N-terminal to C-terminal direction: (I) the 3×IL-2 cytokine; (II) the polypeptide linker; and (III) the IL-2Rβ polypeptide. In some embodiments, the recombinant cytokine receptor comprises an amino acid sequence comprising at least 80%, at least 85%, at least 90%, at least 95%, at least 96%, at least 97%, at least 98%, at least 99% or 100% sequence identity with the amino acid sequence set forth in SEQ ID NO:2. In some embodiments, the recombinant cytokine receptor comprises the 3×IL-2 cytokine having an amino acid sequence comprising at least 90%, at least 95%, at least 96%, at least 97%, at least 98%, at least 99% or 100% sequence identity with the amino acid sequence set forth in SEQ ID NO:5. In some embodiments, the cell is an immune cell. In some embodiments, the cell is a lymphocyte. In some embodiments, the cell is a T cell. In some embodiment, the cell is a Treg.

In some embodiments, the method of treating an immune-related disorder comprises administering a cell (e.g., a Treg cell) comprising a recombinant cytokine receptor comprising an IL-2 cytokine wherein the IL-2 cytokine comprises one or more amino acid substitutions selected from the group consisting of L18R, Q22E, and Q126H. In some embodiments, the method of treating an immune-related disorder comprises administering a cell (e.g., a Treg cell) comprising a recombinant cytokine receptor comprising an IL-2 cytokine wherein the IL-2 cytokine comprises amino acid substitutions L18R, Q22E, and Q126H (“REH IL-2”). In some embodiments, the method of treating an immune-related disorder comprises administering a cell (e.g., a Treg cell) comprising a recombinant cytokine receptor comprising an IL-2 cytokine wherein the IL-2 cytokine comprises amino acid substitutions L18R, Q22E, and Q126H and one or more additional amino acid substitutions. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the method comprises transducing the cell (e.g., Treg cell) with a vector encoding the recombinant cytokine receptor. In some embodiments, the cells are autologous from the individual to be treated. In some embodiments, the REH IL-2 cytokine comprises the amino acid sequence set forth in SEQ ID NO:6. In some embodiments, the REH IL-2 cytokine comprises an amino acid sequence comprising at least 80%, at least 85%, at least 90%, at least 95%, at least 96%, at least 97%, at least 98%, at least 99% or 100% to SEQ ID NO:6. In some embodiments, the method of treating an immune-related disorder comprises administering a cell (e.g., a Treg cell) comprising a recombinant cytokine receptor comprising in N-terminal to C-terminal direction: (I) the REH IL-2 cytokine; (II) the polypeptide linker; and (III) the IL-2Rβ polypeptide. In some embodiments, the recombinant cytokine receptor comprises an amino acid sequence comprising at least 80%, at least 85%, at least 90%, at least 95%, at least 96%, at least 97%, at least 98%, at least 99% or 100% sequence identity with the amino acid sequence set forth in SEQ ID NO:3. In some embodiments, the cell is an immune cell. In some embodiments, the cell is a lymphocyte. In some embodiments, the cell is a T cell. In some embodiment, the cell is a Treg.

In some embodiments, the method of treating an immune-related disorder comprises administering a cell (e.g., a Treg cell) comprising a recombinant cytokine receptor comprising an IL-2 cytokine wherein the IL-2 cytokine comprises one or more amino acid substitutions selected from the group consisting of L18R, Q22E, Q126H, R38D, K43E, and E61R. In some embodiments, the method of treating an immune-related disorder comprises administering a cell (e.g., a Treg cell) comprising a recombinant cytokine receptor comprising an IL-2 cytokine wherein the IL-2 cytokine comprises amino acid substitutions L18R, Q22E, Q126H, R38D, K43E, and E61R (“3×REH IL-2”). In some embodiments, the method of treating an immune-related disorder comprises administering a cell (e.g., a Treg cell) comprising a recombinant cytokine receptor comprising an IL-2 cytokine wherein the IL-2 cytokine comprises amino acid substitutions L18R, Q22E, Q126H, R38D, K43E, and E61R and one or more additional amino acid substitutions. In some embodiments, the recombinant cytokine further comprises one or more amino acid substitutions in the IL-2 cytokine and IL-2Rβ, wherein the IL-2 cytokine comprises one or more amino acid substitutions at position(s) 15, 16, 19, 20, 22, 23, and 81, and the IL-2Rβ polypeptide comprises one or both amino acid substitutions at position(s) 133 and/or 134. For example, in some embodiments, the recombinant cytokine further comprises one or more amino acid substitutions in the IL-2 cytokine and IL-2Rβ, wherein the IL-2 cytokine comprises one or more amino acid substitutions selected from the group consisting of E15S, H16Q, L19V, D20L, Q22K, M23Q, M23A, and R81D, and the IL-2Rβ polypeptide comprises one or both amino acid substitutions H133D and/or Y134F. In some embodiments, the method comprises transducing the cell (e.g., Treg cell) with a vector encoding the recombinant cytokine receptor. In some embodiments, the cells are autologous from the individual to be treated. In some embodiments, the 3×REH IL-2 cytokine comprises the amino acid sequence set forth in SEQ ID NO:12. In some embodiments, the 3×REH IL-2 cytokine comprises an amino acid sequence comprising at least 80%, at least 85%, at least 90%, at least 95%, at least 96%, at least 97%, at least 98%, at least 99% or 100% to SEQ ID NO:12. In some embodiments, the method of treating an immune-related disorder comprises administering a cell (e.g., a Treg cell) comprising a recombinant cytokine receptor comprising in N-terminal to C-terminal direction: (I) the 3×REH IL-2 cytokine; (II) the polypeptide linker; and (III) the IL-2Rβ polypeptide. In some embodiments, the recombinant cytokine receptor comprises an amino acid sequence comprising at least 80%, at least 85%, at least 90%, at least 95%, at least 96%, at least 97%, at least 98%, at least 99% or 100% sequence identity with the amino acid sequence set forth in SEQ ID NO:11. In some embodiments, the cell is an immune cell. In some embodiments, the cell is a lymphocyte. In some embodiments, the cell is a T cell. In some embodiment, the cell is a Treg.

In some embodiments, the recombinant cytokine receptor of the present disclosure is expressed in a T cell. In some embodiments, one or more recombinant cytokine receptors are expressed in a T cell. In some embodiments, the recombinant cytokine receptor is co-expressed with a chimeric antigen receptor (CAR). In some embodiments, the T cell is a regulatory T cell (Treg). In some embodiments, Treg is CD4+, IL-2Rα+, CD127lo. In some embodiments, the Treg expresses FOXP3 and/or HELIOS. In some embodiments, the recombinant cytokine receptor is expressed in a Treg cell.

Immune conditions, diseases, disorders and reactions or responses to be treated according to the methods and compositions of the invention means a disease in which the immune system contributes to pathogenesis or can be part of the treatment. These reactions include, but are not limited to, cancers, inflammation, autoimmune conditions, disorders or diseases and persistent and progressive immune reactions to infectious non-self-antigens from bacterial, viral (e.g., HCV), fungal, or parasitic organisms which invade and persist within mammals and humans. Such conditions and disorders include allergies and/or asthma. The allergies and asthma may be due to sensitization with foreign or non-self-antigens as pollen, animal dander and food proteins. The source of the provoking foreign antigen can be plant. fungal, mold. or other environmental contaminants.

Autoimmunity is defined as persistent and progressive immune reactions to noninfectious self-antigens, as distinct from infectious non-self-antigens from bacterial, viral, fungal, or parasitic organisms which invade and persist within mammals and humans. Autoimmune conditions include graft-versus-host disease, autoimmune polyendocrinopathy syndromes, Type I diabetes mellitus (TIDM), autoimmune gastritis, autoimmune uveoretinitis, autoimmune vasculitis, colitis, thyroiditis, Addison's disease, agammaglobulinemia, alopecia areata, amyloidosis, ankylosing spondylitis, anti-GBM/anti-TBM nephritis, antiphospholipid syndrome, autoimmune hepatitis, autoimmune inner ear disease, axonal and neuronal neuropathy, Behqet's disease, bullous pemphigold, Castleman disease, celiac disease, Chagas disease, chronic inflammatory demyelinating polyneuropathy (CIDP), chronic recurrent multifocal osteomyelitis, Churg-Strauss, cicatrical pemphigold/benign mucosal pemphigold, Cogan's syndrome, cold agglutinin disease, congenital heart block. coxsackie myocarditis. CREST syndrome, Crohn's disease, dermatitis herpetiformis, dermatomyositis, Devic's disease, discoid lupus. Dressier's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome. granulomatosis with polyangiitis, Grave's disease, Guillain-Barre syndrome, Hashimoto's thyroiditis, hemolytic anemia, Henoch-Schonnlien purpura, herpes gestationis or pemphigold gestationis, hypogammaglobulinemia, IgA nephropathy, IgG4-related sclerosing disease, inclusion body myositis, interstitial cystitis, juvenile arthritis, juvenile diabetes (Type 1 diabetes), juvenile myositis, Kawasaki disease, Lambert-Eaton syndrome, leukocytoclastic vasculitis, lichen planus, lichen sclerosis, ligneous conjunctivitis, linear IgA disease, lupus, Lyme disease, chronic Meniere's disease, microscopic polyangiitis, mixed connective tissue disease, Mooren's ulcer, Mucha-Habermann disease, multiple sclerosis, myasthenia gravis, myositis, narcolepsy, neuromyelitis opica, neutropenia, ocular cicatrical pemphigold, optic neuritis, palindromic rheumatism, PANDAS (pediatric autoimmune neuropsychiatric disorders associated with Streptococcus), paraneoplastic cerebellar degeneration, paroxysmal nocturnal hemoglobinuria, Parry Romber syndrome, pars planitis (peripheral uveitis), Parsonnage-Turner syndrome, pemphigus, peripheral neuropathy, perivenous encephalomyelitis, pernicious anemia, POEMS syndrome, polyarteritis nodosa, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, Reiter's syndrome, relapsing polychondritis, restless leg syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic opthalmia, Takayasu's arteritis, temporal arteritis/giant cell arteritis, thrombocytopenic purpura, Tolosa-Hunt syndrome, transverse myelitis. ulcerative colitis, undifferentiated connective tissue disease, uveitis, vasculitis, vitiligo, and Wegner's granulomatosis (granulomatosis with polyangiitis). “Autoantigen” or “self-antigen” as used herein refers to an antigen or epitope which is native to the mammal, and which is immunogenic in said mammalian disease. One aspect of the present application provides a method of treating an immune-related disorder.

The cells may be “allogeneic cells” (allogenicity), which are those isolated from one individual (the donor) and infused into another; whereas “autologous cells” (autology) refer to those cells that are isolated and infused back into the same individual. In some embodiments, the cells are autologous to the individual. In some embodiments, the individual is a human. In some embodiments, the cells are T cells. In some embodiments, the cells are Tregs. In some embodiments, the Treg cells are isolated from human peripheral blood mononuclear cells (PBMCs). In some embodiments, the Treg cells are modified ex vivo with a vector encoding one or more recombinant cytokine receptors. In some embodiments, the Treg cells are expanded ex vivo in the absence of exogenous IL-2. In some embodiments, the Treg cells are modified with a nucleic acid or vector coding for the recombinant cytokine receptors. In some embodiments, the Treg cells are modified to proliferate in the absence of exogenous IL-2. In some embodiments, the Treg cells have one or more markers of IL-2 signaling detected. In some embodiments, the detected marker of IL-2 signaling is phosphorylated STAT5. In some embodiments, the Treg is CD4+, IL-2Rα+, CD127lo. In some embodiments, the Treg expresses FOXP3 and/or HELIOS. In some embodiments, the Treg cells are administered to the same individual to treat an immune-related disorder.

In some embodiments, the method of treating an immune-related disorder comprises administering a Treg cell to an individual in need thereof. In some embodiments, the recombinant cytokine receptor is co-expressed with a chimeric antigen receptor (CAR). In some embodiments, the T cell is a regulatory T cell (Treg). In some embodiments, the Treg is CD4+, IL-2Rα+, CD127lo. In some embodiments, the Treg expresses FOXP3 and/or HELIOS.

In some embodiments, the method of treating an immune-related disorder comprises administering Treg cells that are autologous to the individual. In some embodiments, the individual is a human. In some embodiments, the method of treating an immune-related disorder comprises administering Treg cells that are isolated from human peripheral blood mononuclear cells (PBMCs). In some embodiments, the method of treating an immune-related disorder comprises administering Treg cells that are modified ex vivo with a vector encoding one or more recombinant cytokine receptors. In some embodiments, the method of treating an immune-related disorder comprises administering Treg cells that are expanded ex vivo in the absence of exogenous IL-2. In some embodiments, the method of treating an immune-related disorder comprises administering Treg cells that are modified with a nucleic acid or vector coding for the recombinant cytokine receptors. In some embodiments, the method of treating an immune-related disorder comprises administering the Treg cells are modified to proliferate in the absence of exogenous IL-2. In some embodiments, the method of treating an immune-related disorder comprises administering the Treg cells wherein one or more markers of IL-2 signaling are detected. In some embodiments, the method of treating an immune-related disorder comprises administering the Treg cells wherein the detected marker of IL-2 signaling is phosphorylated STAT5. In some embodiments, the method of treating an immune-related disorder comprises administering the Treg cells which are CD4+, IL-2Rα+, CD127lo. In some embodiments, the method of treating an immune-related disorder comprises administering the Treg cells that express FOXP3 and/or HELIOS. In some embodiments, the method of treating an immune-related disorder comprises administering the Treg cells.

In some embodiments, the method of treating an immune-related disorder comprises administering the Treg cells comprising the recombinant cytokine receptors of the present disclosure. In some embodiments, the suppressive activity of the Tregs on CD8+ and/or CD4+ T cells is increased. In some embodiments, the suppressive activity of the Tregs on CD8+ and/or CD4+ T cells is increased when cultured without exogenous IL-2. In some embodiments, the suppressive activity of the Tregs on CD8+ and/or CD4+ T cells is increased compared to unmodified cells cultured without exogenous IL-2. In some embodiments, the suppressive activity of the transduced Tregs on CD8+ and/or CD4+ T cells cultured without exogenous IL-2 is approximately equivalent or about the same as unmodified Treg cells cultured with IL-2. In some embodiments, the suppressive activity of the transduced Tregs decreases the rate of division of CD4+ and/or CD8+ T cells.

In some embodiments, the method of treating an immune-related disorder comprises transducing a population of cells with a recombinant cytokine receptor. In some embodiments, the method of treating an immune-related disorder comprises transducing a population of T cells with a recombinant cytokine receptor. In some embodiments, the method of treating an immune-related disorder comprises transducing a population of Treg cells with a recombinant cytokine receptor. In some embodiments, the viability of the cells is increased. In some embodiments, at least about 60-99% of the cells in the population of Treg cells transduced with the recombinant cytokine remain viable in vitro approximately 7-20 days after transduction. In some embodiments, at least about 60-99% of the cells in the population of Treg cells transduced with the recombinant cytokine remain viable in vitro approximately 7-14 days after transduction. In some embodiments, at least about 60% of the cells in the population of Treg cells transduced with the recombinant cytokine remain viable in vitro approximately 7 days after transduction.

In some embodiments, the method of treating an immune-related disorder comprises transducing a population of cells with a recombinant cytokine receptor. In some embodiments, the method of treating an immune-related disorder comprises transducing a population of T cells with a recombinant cytokine receptor. In some embodiments, the method of treating an immune-related disorder comprises transducing a population of Treg cells with a recombinant cytokine receptor. In some embodiments, the viability of the cells is increased. In some embodiments, at least about 60-99% of the cells in the population of Treg cells transduced with the recombinant cytokine persist in vivo for at least about 3 days or longer. In some embodiments, at least about 60-90% of the cells in the population of Treg cells transduced with the recombinant cytokine persist in vivo for at least about 3 days or longer. In some embodiments, at least about 60% of the cells in the population of Treg cells transduced with the recombinant cytokine persist in vivo for at least about 3 days or longer. In some embodiments, the population of Treg cells transduced with the recombinant cytokine persist in vivo for at least about any of 1, 2, 3, 5, 6, 8, 10, or 11 months, or at least about a year or more.

In some embodiments, the method of treating an immune-related disorder comprises transducing a population of cells with a recombinant cytokine receptor. In some embodiments, the method of treating an immune-related disorder comprises transducing a population of T cells with a recombinant cytokine receptor. In some embodiments, the method of treating an immune-related disorder comprises transducing a population of Treg cells with a recombinant cytokine receptor, which results in a population of cells that contains more viable cells compared to a composition comprising a population of the same Treg cells not transduced with the recombinant cytokine receptor. In some embodiments, at least about 60-99% of the cells in the population of Treg cells transduced with the recombinant cytokine remain viable in vitro approximately 14-23 days after transduction. In some embodiments, at least about 60-90% of the cells in the population of Treg cells transduced with the recombinant cytokine remain viable in vitro approximately 14-23 days after transduction. In some embodiments, at least about 60% of the cells in the population of Treg cells transduced with the recombinant cytokine remain viable in vitro approximately 14 days after transduction.

In some embodiments, the method of treating an immune-related disorder comprises transducing a population of cells with a recombinant cytokine receptor. In some embodiments, the method of treating an immune-related disorder comprises transducing a population of T cells with a recombinant cytokine receptor. In some embodiments, the method of treating an immune-related disorder comprises transducing a population of Treg cells with a recombinant cytokine receptor, which results in a population of cells that contains more viable cells compared to a composition comprising a population of the same Treg cells not transduced with the recombinant cytokine receptor. In some embodiments, at least about 60-99% of the cells in the population of Treg cells transduced with the recombinant cytokine persist in vivo for at least about 3 days or longer. In some embodiments, at least about 60-90% of the cells in the population of Treg cells transduced with the recombinant cytokine persist in vivo for at least about 3 days or longer. In some embodiments, at least about 60% of the cells in the population of Treg cells transduced with the recombinant cytokine persist in vivo for at least about 3 days or longer. In some embodiments, the population of Treg cells transduced with the recombinant cytokine persist in vivo for at least about any of 1, 2, 3, 5, 6, 8, 10, or 11 months, or at least a year or more.

In some embodiments, the method of treating an immune-related disorder comprises transducing a population of cells with a recombinant cytokine receptor. In some embodiments, the method of treating an immune-related disorder comprises transducing a population of T cells with a recombinant cytokine receptor. In some embodiments, the method of treating an immune-related disorder comprises transducing a population of Treg cells with a recombinant cytokine receptor, which results in a population of cells that contains more viable cells compared to a composition comprising a population of the same Treg cells not transduced with the recombinant cytokine receptor. In some embodiments, at least about 60-99% of the cells in the population of Treg cells transduced with the recombinant cytokine persist in vivo for at least about 14 days or longer. In some embodiments, at least about 60-90% of the cells in the population of Treg cells transduced with the recombinant cytokine persist in vivo for at least about 14 days or longer. In some embodiments, at least about 60% of the cells in the population of Treg cells transduced with the recombinant cytokine persist in vivo for at least about 14 days or longer. In some embodiments, the population of Treg cells transduced with the recombinant cytokine persist in vivo for at least about any of 1, 2, 3, 5, 6, 8, 10, or 11 months, or at least a year or more.

In some embodiments, the method of treating an immune-related disorder comprises transducing a population of cells with a recombinant cytokine receptor. In some embodiments, the method of treating an immune-related disorder comprises transducing a population of T cells with a recombinant cytokine receptor. In some embodiments, the method of treating an immune-related disorder comprises transducing a population of Treg cells with a recombinant cytokine receptor. which results in a population of cells that contains more viable cells compared to a composition comprising a population of the same Treg cells not transduced with the recombinant cytokine receptor. In some embodiments, at least about 60-99% of the cells in the population of Treg cells transduced with the recombinant cytokine persist in vivo for at least about 23 days or longer. In some embodiments, at least about 60-90% of the cells in the population of Treg cells transduced with the recombinant cytokine persist in vivo for at least about 23 days or longer. In some embodiments, at least about 60% of the cells in the population of Treg cells transduced with the recombinant cytokine persist in vivo for at least about 23 days or longer. In some embodiments, the population of Treg cells transduced with the recombinant cytokine persist in vivo for at least about any of 1, 2, 3, 5, 6, 8, 10, or 11 months, or at least a year or more.

In some embodiments, the method of treating an immune-related disorder comprises transducing a population of Treg cells transduced with the recombinant cytokine receptor, wherein the population of transduced Treg cells expands at least two-fold more than a population of the same Treg cells not transduced with the recombinant cytokine receptor. In some embodiments, the method of treating an immune-related disorder comprises a composition wherein the population of Treg cells transduced with the recombinant cytokine receptor expands at least two-fold, and wherein the population of Treg cells transduced with the recombinant cytokine receptor maintains expression of at least one Treg marker selected from the group consisting of CD4+, IL-2Rα+, and CD127lo. In some embodiments, the Treg marker may also consist of FOXP3 and/or HELIOS.

In some embodiments, the recombinant cytokine receptors of the present disclosure may also be used in combination with a chimeric antigen receptor (CAR). In some embodiments, the modified Treg comprises a recombinant cytokine receptor and a chimeric antigen.

### VII. Method of Expanding a Transduced Treg Cell

In some embodiments, the method of expanding a transduced T cell comprising a recombinant cytokine receptor does not activate IL-2 signaling on a T cell that does not comprise the recombinant cytokine receptor. In some embodiments, the method of expanding a transduced T cell comprising the recombinant cytokine receptor does not activate IL-2 signaling on Treg cells that do not comprise the recombinant cytokine receptor.

In some embodiments the method of expanding a transduced T cell comprising the recombinant cytokine receptor does not activate signaling of another, different IL-2 receptor. In some embodiments, the method of expanding a transduced T cell comprising the recombinant cytokine receptor does not cause the transduced Treg cell to secrete one or more cytokines more than a Treg cell cultured with exogenous IL-2 cytokine. In some embodiments, the method of expanding a transduced T cell comprising the recombinant cytokine receptor does not cause the transduced Treg cell to secrete one or more cytokines at a level higher than the level of cytokine secretion by a Treg cell cultured with exogenous IL-2 cytokine.

In some embodiments, the method of expanding a transduced T cell comprising the recombinant cytokine receptor allows the transduced Treg cell to proliferate without exogenous IL-2. In some embodiments, the method of expanding a transduced T cell comprises the method wherein the relative amount of Treg cells in a composition comprising a population of Treg cells transduced with the recombinant cytokine receptor increases over time. In some embodiments, the method of expanding a transduced T cell comprises the method wherein the population of Treg cells transduced with the recombinant cytokine receptor grown without IL-2 contains a similar number of viable cells compared to a composition comprising a population of the same Treg cells not transduced with the recombinant cytokine receptor grown with IL-2. In some embodiments, the method of expanding a transduced T cell comprises the method wherein at least about 80% of the population of Treg cells transduced with the recombinant cytokine maintain FOXP3 and/or HELIOS expression about 14 days after transduction. In some embodiments, the method of expanding a transduced T cell comprises the method wherein at least about 80% of the cells in the population of Treg cells transduced with the recombinant cytokine maintain FOXP3 and/or HELIOS expression about 23 days after transduction.

In some embodiments, the method of expanding a transduced T cell comprises the method wherein the population of Treg cells transduced with the recombinant cytokine receptor expands at least two-fold more than a population of the same Treg cells not transduced with the recombinant cytokine receptor. In some embodiments, the method of expanding a transduced T cell comprises the method wherein the population of Treg cells transduced with the recombinant cytokine receptor expands at least two-fold, and wherein the population of Treg cells transduced with the recombinant cytokine receptor maintains expression of least one Treg marker selected from the group consisting of CD25+, FOXP3, and HELIOS.

In some embodiments, the method of expanding a transduced T cell comprises the method wherein the relative amount of Treg cells in a composition comprising a population of Treg cells transduced with the recombinant cytokine receptor increases over time when cultured without IL-2.

### VIII. Definitions

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps. reagents, or conditions are specifically embraced by the present disclosure and are disclosed herein just as if each and every combination was individually and explicitly disclosed.

As used herein “IL-2 cytokine” includes both “wild-type” IL-2 (SEQ ID NO:4) as well as IL-2 mutants comprising one or more amino acid substitutions (e.g., amino acid substitutions L18R, Q22E, and Q126H; and/or R38D, K43E, and E61R; SEQ ID NOs: 5, 6, and 12).

IL-2RA is used interchangeably herein with IL-2Rα.

IL-2RB is used interchangeably herein with IL-2Rβ.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X”.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

As used herein, the terms “including,” “containing,” and “comprising” are used in their open, non-limiting sense.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, California. The ALIGN-2 program should be compiled for use on a UNIX operating system, preferably digital UNIX V4.0D. All sequence comparison parameters are set by the ALIGN-2 program and do not vary.

An “individual” or “subject” is a mammal. Mammals include, but are not limited to, domesticated animals (e.g., cows, sheep, cats, dogs, and horses), primates (e.g., humans and non-human primates such as rhesus and cynomolgus monkeys), rabbits, and rodents (e.g., mice and rats). In some embodiments, the individual or subject is a human.

Treatment” or “therapy” of a subject refers to any type of intervention or process performed on, or the administration of an active agent to, the subject with the objective of curing, reversing, alleviating, ameliorating, inhibiting, slowing down, or preventing the onset, progression, development, severity, or recurrence of a symptom, complication, condition, or biochemical indicia associated with a disease.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The phrase “pharmaceutically acceptable” indicates that the substance or composition must be compatible chemically and/or toxicologically, with the other ingredients comprising a formulation, and/or the mammal being treated therewith.

As described herein, any concentration range, percentage range, ratio range, or integer range is to be understood to include the value of any integer within the recited range and, when appropriate, fractions thereof (such as one tenth and one hundredth of an integer), unless otherwise indicated. Description of endpoints includes ranges between all endpoints disclosed. For example, description of 1, 2, or 3 includes the ranges 1-2, 2-3 and 1-3.

### EMBODIMENTS

1. A recombinant cytokine receptor comprising:
an IL-2 cytokine;
an IL-2 receptor extracellular domain;
a transmembrane domain; and
an IL-2 receptor intracellular domain;
wherein the IL-2 receptor extracellular domain is tethered to the IL-2 cytokine thereof by a polypeptide linker.

2. The recombinant cytokine receptor of embodiment 1, wherein the IL-2 receptor extracellular domain comprises the extracellular domain of IL-2Rβ.

3. The recombinant cytokine receptor of embodiments 1-2, wherein the IL-2 cytokine comprises at least one amino acid substitution that reduces affinity for IL-2Rα and/or IL-2Rγ by at least about two-fold.

4. The recombinant cytokine receptor of any one of embodiments 1-3, wherein the recombinant cytokine receptor forms a protein complex with IL-2Rγ.

5. The recombinant cytokine receptor of any one of embodiments 1-4, wherein the recombinant cytokine receptor engages in IL-2 signaling in the absence of exogenous IL-2.

6. The recombinant cytokine receptor of any one of embodiments 1-5, wherein the IL-2 cytokine comprises one or more amino acid substitutions selected from the amino acid positions 18, 22, 126, 38, 43, 61, 15, 16, 19, 20, 22, 23, and 81.

7. The recombinant cytokine receptor of any one of embodiments 1-6. wherein the IL-2 cytokine comprises one or more amino acid substitutions selected from the group consisting of L18R, Q22E, Q126H, R38D, K43E, E61R, E15S, H16Q, L19V, D20L, Q22K, M23Q, M23A, and R81D.

8. The recombinant cytokine receptor of any one of embodiments 1-7, wherein the IL-2 cytokine comprises the amino acid substitutions L18R, Q22E, and Q126H; and/or R38D, K43E, and E61R; and/or E15S, H16Q, L19V, D20L, M23Q, and R81D; and/or E15S. H16Q, L19V, D20L, Q22K, and M23A.

9. The recombinant cytokine receptor of any one of embodiments 1-8, wherein the IL-2 cytokine comprises an amino acid sequence comprising at least 80%, at least 85%, at least 90%, at least 95%, at least 96%, at least 97%, at least 98%, at least 99% or 100% sequence identity with the amino acid sequence selected from the group consisting of SEQ ID NOs:4-6 and 12.

10. The recombinant cytokine receptor of any one of embodiments 1-9, comprising:
a. a WT IL-2 cytokine, a polypeptide linker, an IL-2Rβ extracellular domain, an IL-2Rβ transmembrane domain, and an IL-2Rβ intracellular domain;
b. a 3×IL-2 cytokine, a polypeptide linker, an IL-2Rβ extracellular domain, an IL-2Rβ transmembrane domain, and an IL-2Rβ intracellular domain;
c. an REH IL-2 cytokine, a polypeptide linker, an IL-2Rβ extracellular domain, an IL-2Rβ transmembrane domain, and an IL-2Rβ intracellular domain; or
d. a 3×REH IL-2 cytokine, a polypeptide linker, an IL-2Rβ extracellular domain, an IL-2Rβ transmembrane domain, and an IL-2Rβ intracellular domain;
e. an IL-2 cytokine comprising substitutions at positions E15S, H16Q, L19V, D20L, M23Q, and R81D, a polypeptide linker, an IL-2Rβ extracellular domain comprising substitutions at positions H133D and Y134F, an IL-2Rβ transmembrane domain, and an IL-2Rβ intracellular domain; or
f. an IL-2 cytokine comprising substitutions at positions E15S, H16Q, L19V, D20L, Q22K, and M23A, a polypeptide linker, an IL-2Rβ extracellular domain comprising substitutions at positions H133D and Y134F, an IL-2Rβ transmembrane domain, and an IL-2Rβ intracellular domain.

11. The recombinant cytokine receptor of any one of embodiments 1-10, comprising:
a. an IL-2 cytokine comprising the amino acid sequence of SEQ ID NO:4, a polypeptide linker, an IL-2Rβ extracellular domain, an IL-2Rβ transmembrane domain, and an IL-2Rβ intracellular domain;
b. an IL-2 cytokine comprising the amino acid sequence of SEQ ID NO:5, a polypeptide linker, an IL-2Rβ extracellular domain, an IL-2Rβ transmembrane domain, and an IL-2Rβ intracellular domain;
c. an IL-2 cytokine comprising the amino acid sequence of SEQ ID NO:6, a polypeptide linker, an IL-2Rβ extracellular domain, an IL-2Rβ transmembrane domain, and an IL-2Rβ intracellular domain; or
d. an IL-2 cytokine comprising the amino acid sequence of SEQ ID NO:12, a polypeptide linker, an IL-2Rβ extracellular domain, an IL-2Rβ transmembrane domain, and an IL-2Rβ intracellular domain.

12. The recombinant cytokine receptor of any one of embodiments 1-11, wherein the IL-2 receptor beta extracellular domain comprises at least 80%, at least 85%, at least 90%, at least 95%, at least 96%, at least 97%, at least 98%, at least 99% or 100% sequence identity with the amino acid sequence set forth in SEQ ID NO:13.

13. The recombinant cytokine receptor of any one of embodiments 1-12, wherein the IL-2 receptor beta transmembrane domain comprises at least 80%, at least 85%, at least 90%, at least 95%, at least 96%, at least 97%, at least 98%, at least 99% or 100% sequence identity with the amino acid sequence set forth in SEQ ID NO:14.

14. The recombinant cytokine receptor of any one of embodiments 1-13, wherein the IL-2 receptor beta intracellular domain comprises at least 80%, at least 85%, at least 90%, at least 95%, at least 96%, at least 97%, at least 98%, at least 99% or 100% sequence identity with the amino acid sequence set forth in SEQ ID NO:15.

15. The recombinant cytokine receptor of any one of embodiments 1-14, wherein the polypeptide linker comprises glycines and serines.

16. The recombinant cytokine receptor of any one of embodiments 1-14, wherein the polypeptide linker comprises an amino acid sequence selected from the group consisting of SEQ ID NOs:9 and 16-21.

17. The recombinant cytokine receptor of any one of embodiments 1-16, comprising:
a. an IL-2 cytokine comprising the amino acid sequence of SEQ ID NO:4, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15;
b. an IL-2 cytokine comprising the amino acid sequence of SEQ ID NO:5, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15;
c. an IL-2 cytokine comprising the amino acid sequence of SEQ ID NO:6, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15; or
d. an IL-2 cytokine comprising the amino acid sequence of SEQ ID NO:12, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15.

18. The recombinant cytokine receptor of any of one embodiments 1-17, wherein the recombinant cytokine receptor comprises an amino acid sequence comprising at least 80%, at least 85%, at least 90%, at least 95%, at least 96%, at least 97%, at least 98%, at least 99% or 100% sequence identity with the amino acid sequence selected from the group consisting of SEQ ID NOs:1-3 and 11.

19. The recombinant cytokine receptor of any one of embodiments 1-18, consisting of:
an IL-2 cytokine;
an IL-2 receptor beta extracellular domain;
a transmembrane domain; and
an IL-2 receptor beta intracellular domain;
   wherein the IL-2 receptor beta extracellular domain is tethered to the IL-2 cytokine thereof by a polypeptide linker.

20. A nucleic acid encoding the recombinant cytokine receptor of any one of embodiments 1-19.

21. A vector comprising the nucleic acid of embodiment 20.

22. The vector of embodiment 21, wherein the vector is a lentiviral vector.

23. The vector of embodiment 21 or embodiment 22, further comprising a marker gene.

24. The vector of embodiment 23, wherein the marker gene is a transmembrane protein.

25. The vector of embodiment 24, wherein the transmembrane protein is EGFR.

26. A T cell comprising the recombinant cytokine receptor of any one of embodiments 1-19, the nucleic acid of embodiment 20, or the vector of any one of embodiments 21-25.

27. The T cell of embodiment 26, wherein the T cell is a regulatory T cell (Treg), wherein the Treg is CD25+ and expresses FOXP3 and/or HELIOS.

28. The T cell of embodiment 26 or embodiment 27. further comprising a chimeric antigen receptor (CAR).

29. A composition comprising the nucleic acid of embodiment 20, the vector of any one of embodiments 21-25, or the T cell of any one of embodiments 26-28.

30. A method of treating an immune related disorder comprising administering the T cell of any one of embodiments 26-28 or the composition of embodiment 23, to an individual in need thereof.

31. The method of embodiment 30, wherein the cells are autologous to the individual.

32. The method of embodiment 30 or embodiment 31, wherein the individual is human.

33. A method of expanding a transduced Treg cell in the absence of exogenous IL-2 comprising introducing the nucleic acid of embodiment 20, or the vector of any one of embodiments 21-25 into the Treg cell and culturing the cell.

34. The method of claim 33, wherein the recombinant cytokine receptor does not activate IL-2 signaling on a cell that does not comprise the recombinant cytokine receptor.

35. The method of embodiment 33 or embodiment 34, wherein the recombinant cytokine receptor does not activate IL-2 signaling on Tregs that do not comprise the recombinant cytokine receptor.

36. The method of any one of embodiments 33-35, where the recombinant cytokine receptor does not activate signaling of an IL-2 receptor comprising a different amino acid sequence.

37. The method of any one of embodiments 33-36, where the recombinant cytokine receptor does not cause the transduced Treg cell to secrete one or more cytokines more than a Treg cell cultured with exogenous IL-2 cytokine.

38. The method of any one of embodiments 33-37, further comprising detecting at least one Treg marker selected from the group consisting of CD4+, CD25+, and CD127lo.

39. The method of any one of embodiments 33-38, further comprising detecting FOXP3 and/or HELIOS.

40. The method of any one of embodiments 35-39, further comprising detecting methylation at Treg-specific differentially regulated genes.

41. The method of embodiment 40, wherein the Treg-specific differentially regulated gene is FOXP3.

42. The method of any one of embodiments 33-41, wherein the Treg cell is able to proliferate in the absence of IL-2.

43. The method of any one of embodiments 33-42, wherein one or more markers of IL-2 signaling is detected.

44. The method of embodiment 43, wherein the one or more markers of IL-2 signaling comprises phosphorylated STAT5.

45. The method of any one of embodiments 33-44, wherein suppressive activity of the Treg on CD8+ and/or CD4+ T cells is increased.

46. The method of embodiment 45, wherein suppressive activity comprises a decreased CD4+ and/or CD8+ T cell rate of division.

47. The method of any one of embodiments 33-46, wherein the relative amount of Treg cells in a composition comprising a population of Treg cells transduced with the recombinant cytokine receptor increases over time.

48. The method of any one of embodiments 33-47, wherein a composition comprising a population of Treg cells transduced with the recombinant cytokine receptor grown without IL-2 contains a similar number of viable cells compared to a composition comprising a population of the same Treg cells not transduced with the recombinant cytokine receptor grown with IL-2.

49. The method of embodiment 47 or embodiment 48, wherein at least 80% of the cells in the population of Treg cells transduced with the recombinant cytokine maintain FOXP3 and/or HELIOS expression 14 days after transduction.

50. The method of embodiment 47 or embodiment 48, wherein at least 80% of the cells in the population of Treg cells transduced with the recombinant cytokine maintain FOXP3 and/or HELIOS expression 23 days after transduction.

51. The method of any one of embodiments 33-50, further comprising culturing the Treg cell in a composition comprising a cytokine other than IL-2.

52. The method of any one of embodiments 33-51, wherein the population of Treg cells transduced with the recombinant cytokine receptor expands at least two-fold more than a population of the same Treg cells not transduced with the recombinant cytokine receptor.

53. The method of any one of embodiments 33-52, wherein the population of Treg cells transduced with the recombinant cytokine receptor expands at least two-fold, and wherein the population of Treg cells transduced with the recombinant cytokine receptor maintains expression of least one Treg marker selected from the group consisting of CD25+, FOXP3, and HELIOS.

54. The method of any one of embodiments 33-44, 51, and 53, wherein the method generates Treg cells comprising a recombinant cytokine receptor, wherein:
i. the in vitro and/or in vivo suppressive activity of the Treg on CD8+ and/or CD4+ T cells is increased compared to a control Treg that is not transduced with the recombinant cytokine receptor,
ii. the rate of division of CD4+ and/or CD8+ T cells is decreased when cultured in the presence of the Treg compared to the rate of division of CD4+ and/or CD8+ T cells when cultured without the Treg;
iii. the relative amount of Treg cells in a composition comprising a population of Treg cells transduced with a recombinant cytokine receptor increases over time;
iv. a composition comprising a population of Treg cells transduced with the recombinant cytokine receptor grown without IL-2 contains a similar number of viable cells compared to a composition comprising a population of the same Treg cells not transduced with the recombinant cytokine receptor grown with IL-2;
v. at least 80% of the cells in the population of Treg cells transduced with the recombinant cytokine receptor maintain FOXP3 and/or HELIOS expression about 14 days after transduction;
vi. at least 80% of the cells in the population of Treg cells transduced with the recombinant cytokine receptor maintain FOXP3 and/or HELIOS expression about 23 days after transduction;
vii. the population of Treg cells transduced with the recombinant cytokine receptor expands at least two-fold more than a population of the same Treg cells not transduced with the recombinant cytokine receptor,
viii. the IL-10 cytokine levels produced by the Treg in vitro and/or in vivo are equivalent or increased compared to a control Treg that is not transduced with the recombinant cytokine receptor,
ix. the IFN-γ cytokine levels produced by the Treg in vitro and/or in vivo are equivalent or increased compared to a control Treg that is not transduced with the recombinant cytokine receptor,
x. the Gr-B cytokine levels produced by the Treg in vitro and/or in vivo are equivalent or increased compared to a control Treg that is not transduced with the recombinant cytokine receptor;
xi. the Treg functionally re-activates to higher levels than control Tregs that are not transduced with the recombinant cytokine receptor in vitro and/or in vivo; and/or
xii. the level of Treg proliferation increases from re-stimulation in vitro and/or in vivo one or more times.

55. The T cell of any one of embodiments 20-22 for use in treating an immune related disorder in an individual.

56. The T cell for use of embodiment 48, wherein the T cell is a Treg cell.

57. The T cell for use of embodiment 48 or embodiment 49, wherein the T cell is autologous to the individual.

58. The T cell for use of any one of embodiments 48-50, wherein the individual is human.

59. The T cell for use of any of embodiments 48-51, wherein the T cell prevents, ameliorates, or cures an immune-related disorder.

60. The use of the T cell of any one of embodiments 20-22 in the manufacture of a medicament for treating an immune-related disorder in an individual.

61. The use of embodiment 53, wherein the T cell is a Treg cell.

62. The use of embodiment 53 or embodiment 54, wherein the T cell is autologous to the individual.

63. The use of any one of embodiments 53-55. wherein the individual is human.

64. The use of any of embodiments 53-56, wherein the medicament prevents, ameliorates, or cures an immune-related disorder.

65. The T cell for use of any one of embodiments 55-59, wherein the individual expresses IL-2.

66. The use of any one of embodiments 60-64, wherein the individual expresses IL-2.

67. The method of any one of embodiments 30-32, wherein the method further prevents, ameliorates, or cures an immune-related disorder.

68. The method of any one of embodiments 30-32 and 67, wherein the individual expresses IL-2.

69. A regulatory T cell (Treg) comprising a recombinant cytokine receptor, wherein the recombinant cytokine receptor comprises:
an IL-2 cytokine;
an IL-2 receptor beta extracellular domain;
a transmembrane domain; and
an IL-2 receptor beta intracellular domain;
   wherein the IL-2 receptor beta extracellular domain is tethered to the IL-2 cytokine by a polypeptide linker.

70. The Treg of embodiment 69, wherein the recombinant cytokine receptor forms a protein complex with IL-2Rγ.

71. The Treg of embodiment 69 or embodiment 70, wherein the recombinant cytokine receptor engages in IL-2 signaling in the absence of exogenous IL-2.

72. The Treg of any one of embodiments 69-71, wherein the IL-2 cytokine comprises at least one amino acid substitution that reduces affinity for IL-2Rα and/or IL-2Rγ by at least about two-fold.

73. The Treg of any one of embodiments 69-72, wherein the IL-2 cytokine comprises one or more amino acid substitutions selected from the amino acid positions 18, 22, 126, 38, 43, 61, 15, 16, 19, 20, 22, 23, and 81.

74. The Treg of any one of embodiments 69-73, wherein the IL-2 cytokine comprises one or more amino acid substitutions selected from the group consisting of L18R, Q22E, Q126H, R38D, K43E, E61R, E15S, H16Q, L19V, D20L, Q22K, M23Q, M23A, and R81D.

75. The Treg of any one of embodiments 69-74, wherein the IL-2 cytokine comprises the amino acid substitutions L18R, Q22E, and Q126H; and/or R38D, K43E, and E61R; and/or E15S, H16Q, L19V, D20L, M23Q, and R81D; and/or E15S, H16Q, L19V, D20L, Q22K, and M23A.

76. The Treg of any one of embodiments 69-75, comprising:
a. a WT IL-2 cytokine, a polypeptide linker, an IL-2Rβ extracellular domain, an IL-2Rβ transmembrane domain, and an IL-2Rβ intracellular domain;
b. a 3×IL-2 cytokine, a polypeptide linker, an IL-2Rβ extracellular domain, an IL-2Rβ transmembrane domain, and an IL-2Rβ intracellular domain;
c. an REH IL-2 cytokine, a polypeptide linker, an IL-2Rβ extracellular domain, an IL-2Rβ transmembrane domain, and an IL-2Rβ intracellular domain;
d. a 3×REH IL-2 cytokine, a polypeptide linker, an IL-2Rβ extracellular domain, an IL-2Rβ transmembrane domain, and an IL-2Rβ intracellular domain;
e. an IL-2 cytokine comprising substitutions at positions E15S, H16Q, L19V, D20L, M23Q, and R81D, a polypeptide linker, an IL-2Rβ extracellular domain comprising substitutions at positions H133D and Y134F, an IL-2Rβ transmembrane domain, and an IL-2Rβ intracellular domain; or
f. an IL-2 cytokine comprising substitutions at positions E15S, H16Q, L19V, D20L, Q22K, and M23A, a polypeptide linker, an IL-2Rβ extracellular domain comprising substitutions at positions H133D and Y134F, an IL-2Rβ transmembrane domain, and an IL-2Rβ intracellular domain.

77. The Treg of any one of embodiments 69-76, wherein the polypeptide linker comprises glycines and serines.

78. The Treg of any one of embodiments 69-77, comprising:
a. an IL-2 cytokine comprising the amino acid sequence of SEQ ID NO:4, a polypeptide linker, an IL-2Rβ extracellular domain, an IL-2Rβ transmembrane domain, and an IL-2Rβ intracellular domain;
b. an IL-2 cytokine comprising the amino acid sequence of SEQ ID NO:5, a polypeptide linker, an IL-2Rβ extracellular domain, an IL-2Rβ transmembrane domain, and an IL-2Rβ intracellular domain;
c. an IL-2 cytokine comprising the amino acid sequence of SEQ ID NO:6, a polypeptide linker, an IL-2Rβ extracellular domain, an IL-2Rβ transmembrane domain, and an IL-2Rβ intracellular domain; or
d. an IL-2 cytokine comprising the amino acid sequence of SEQ ID NO:12, a polypeptide linker, an IL-2Rβ extracellular domain, an IL-2Rβ transmembrane domain, and an IL-2Rβ intracellular domain.

79. The Treg of any one of embodiments 69-78, wherein the recombinant cytokine receptor comprises:
a. an IL-2 cytokine comprising an amino acid sequence comprising at least 80%, at least 85%, at least 90%, at least 95%, at least 96%, at least 97%, at least 98%, at least 99% or 100% sequence identity with the amino acid sequence selected from the group consisting of SEQ ID NOs:4-6 and 12;
b. a polypeptide linker comprising an amino acid sequence selected from the group consisting of SEQ ID NOs:9 and 16-21;
c. an IL-2 receptor beta extracellular domain comprising at least 80%, at least 85%, at least 90%, at least 95%, at least 96%, at least 97%, at least 98%, at least 99% or 100% sequence identity with the amino acid sequence set forth in SEQ TD NO:13;
d. an IL-2 receptor beta transmembrane domain comprising at least 80%, at least 85%, at least 90%, at least 95%, at least 96%, at least 97%, at least 98%, at least 99% or 100% sequence identity with the amino acid sequence set forth in SEQ ID NO:14; and/or
e. an IL-2 receptor beta intracellular domain comprises at least 80%, at least 85%, at least 90%, at least 95%, at least 96%, at least 97%, at least 98%, at least 99% or 100% sequence identity with the amino acid sequence set forth in SEQ ID NO:15.

80. The Treg of any one of embodiments 69-79, wherein the IL-2 cytokine comprises an amino acid sequence comprising at least 80%, at least 85%, at least 90%, at least 95%, at least 96%, at least 97%, at least 98%, at least 99% or 100% sequence identity with the amino acid sequence set forth in SEQ ID NO:4.

81. The Treg of any one of embodiments 69-80, wherein the recombinant cytokine receptor does not comprise a T cell receptor activation domain or a T cell costimulatory domain, optionally wherein the T cell receptor activation domain or T cell costimulatory domain is a CD28 signaling domain.

82. The Treg of any one of embodiments 69-81, comprising:
a. an IL-2 cytokine comprising the amino acid sequence of SEQ ID NO:4, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15;
b. an IL-2 cytokine comprising the amino acid sequence of SEQ ID NO:5, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO: 14, and an IL-2Rβ intracellular domain comprising the amino acid sequence of SEQ ID NO:15;
c. an IL-2 cytokine comprising the amino acid sequence of SEQ ID NO:6, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15; or
d. an IL-2 cytokine comprising the amino acid sequence of SEQ ID NO:12, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15.

83. The Treg of any one of embodiments 69-82, wherein the recombinant cytokine receptor comprises an amino acid sequence comprising at least 80%, at least 85%, at least 90%, at least 95%, at least 96%, at least 97%, at least 98%, at least 99% or 100% sequence identity with the amino acid sequence selected from the group consisting of SEQ ID NOs:1-3 and 11.

84. The Treg of any one of embodiments 69-83, comprising a recombinant cytokine receptor consisting of:
an IL-2 cytokine;
an IL-2 receptor beta extracellular domain;
a transmembrane domain; and
an IL-2 receptor beta intracellular domain;
   wherein the IL-2 receptor beta extracellular domain is tethered to the IL-2 cytokine thereof by a polypeptide linker.

85. The Treg of any one of embodiments 69-84, wherein the Treg is CD25+ and expresses FOXP3 and/or HELIOS.

86. The Treg of any one of embodiments 69-85, further comprising a chimeric antigen receptor (CAR).

87. The Treg of any one of embodiments 69-86 for use in treating an immune related disorder in an individual.

88. The Treg for use of embodiment 87, wherein the Treg is autologous to the individual.

89. The Treg for use of embodiments 87 or embodiment 88, wherein the individual is human.

90. The Treg for use of any of embodiments 87-89, wherein the T cell prevents, ameliorates, or cures an immune-related disorder.

91. The method of any one of embodiments 87-90, wherein the individual expresses IL-2.

92. The recombinant cytokine receptor of any one of embodiments 1-18, wherein the IL-2 cytokine comprises an amino acid sequence comprising at least 80%, at least 85%, at least 90%, at least 95%, at least 96%, at least 97%, at least 98%, at least 99% or 100% sequence identity with the amino acid sequence set forth in SEQ ID NO:4.

93. The recombinant cytokine receptor of any one of embodiments 1-18 and 92, wherein the recombinant cytokine receptor does not comprise a T cell receptor activation domain or a T cell costimulatory domain, optionally wherein the T cell receptor activation domain or T cell costimulatory domain is a CD28 signaling domain.

### EXAMPLES

### Example 1: The CD-122 Domain of the IL-2 Receptor is Tethered to IL-2 and IL-2 Mutants to Form a Recombinant Cytokine Receptor

Treg cells are a unique lineage within CD4+ and CD8+ T cells and thus express a unique gene profile and protein markers. Traditionally, Tregs have been identified from broader T cell populations by IL-2Rα, FOXP3, and HELIOS. Using FACS analysis, tethered IL-2 mutants/IL-2Rβ recombinant cytokine receptors displayed increased Treg-specific lineage markers in the absence of IL-2 cytokine. These data indicate that the expression of the recombinant cytokine receptors supports the Treg phenotype in environments that are limited or lacking in IL-2.

Human regulatory T cells were engineered to express chimeric IL-2 recombinant cytokine receptors that are tethered to interleukin 2 (IL-2) and IL-2 mutants (3×IL-2, REH IL-2, 3×REH IL-2). REH IL-2 mutant is an IL-2 cytokine with the amino acid substitutions L18R, Q22E, and Q126H. 3×IL-2 mutant is an IL-2 cytokine with the amino acid substitutions R38D, K43E, and E61R. 3×REH IL-2 is an IL-2 cytokine with the amino acid substitutions L18R, Q22E, Q127H, R38D, K43E, and E61R.

Lentiviral construct production: Recombinant cytokine receptor constructs were packaged as lentiviral particles for transduction into cells of interest. HEK 293 FT suspension cells (Invitrogen) were seeded at 4.7×10⁶/mL and transfected with transfer and packaging plasmids (Aldevron pALD-VSV-G, pALD-GagPol, pALD-Rev) using LV_MAX transfection reagent as per manufacturer's protocol (Gibco). Viral supernatant was collected and filtered through a 0.45 μm polyvinylidene difluoride filter to remove cell debris. The viral supernatant was centrifuged at 10,000×g overnight to concentrate viral vectors. After 12-16 h, supernatant was removed, and the viral vector pellet was resuspended in Opti-MEM media. Resuspended viral vector was aliquoted and stored at −80° C.

Functional titer assay: Functional titers were assessed to identify transduction conditions for experiments. SupT1 cells (ATCC) were cultured in RPMI medium supplemented with 10% fetal bovine serum. On the day of transduction, 50 μL of SupT1 cells at 4×10⁵ cells/mL were added to each well of a 96-well plate. Lentivirus stock was thawed at room temperature and diluted 1:20 in culture medium. Subsequently, a three-fold serial dilution was performed from 60 to 393,660-fold in culture medium. 50 μL of diluted lentivirus were added to a 96-well plate containing 50 μL of cells. On day 2 of transduction, 100 μL of culture media was added. On day 3, transduced cells were harvested and stained with appropriate antibody to detect surface markers. Labeled cells were analyzed by flow cytometry. Untransduced SupT1 cells were used as negative control to set a gate for flow cytometry analysis. Viral dilution that produced between 5-20% maker positive cells were used for the calculation of functional titer, based on the following formula:

$$Titer ( TU / mL ) = \frac{{[ Cell \# ]}_{transduced} \times {[ ( \% ) cells ]}_{transduced} \times [ dilution factor ]}{{[ ( mL ) volume ]}_{transduced}} .$$

Treg isolation and culturing: Control Tregs (i.e., Tregs that were transduced with the MND-expressing empty vector that lacked the recombinant cytokine receptor construct) were cultured with exogenous IL-2. Tregs transfected with IL-2 tethered constructs were cultured without exogenous IL-2.

FACS analysis: Treg cells were cultured and fixed, then washed with 1×RoboSep Buffer and permeabilized. Tregs were subsequently washed twice and stained in 1×RoboSep Buffer for the detection of various markers. Stained cells underwent FACS analysis.

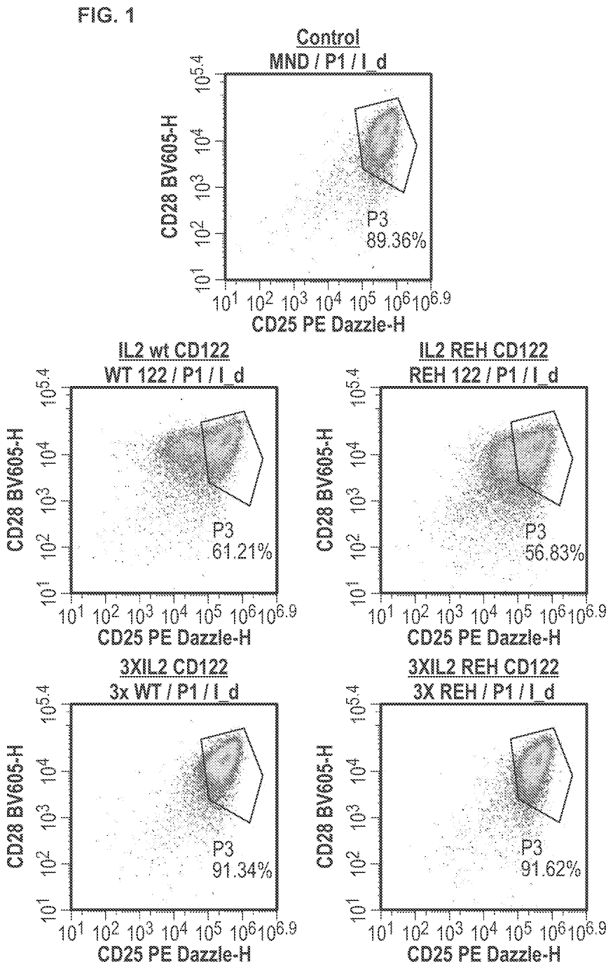

Results: Treg cells transfected with either WT IL-2-IL-2Rβ or IL-2 REH-IL-2Rβ resulted in approximately 60% IL-2Rα+ cells (FIG. 1). Tregs transfected with IL-2 3× and IL-2 3×-REH constructs result in approximately 90% IL-2Rα+ cells (FIG. 1). These levels of IL-2Rα+ cells were comparable to the positive control. 3×IL-2/IL-2Rβ and 3×REH IL-2/IL-2Rβ increased IL-2Rα expression in Tregs whereas WT IL-2/IL-2Rβ and the REH IL-2/IL-2Rβ did not.

### Example 2: IL-2 Mutants Tethered to the IL-2Rβ Domain of the IL-2 Receptor Promote Treg Growth and Identity

Example 2 shows that cells express the 3×IL-2/IL-2Rβ and the 3×REH IL-2/IL-2Rβ recombinant cytokine receptor constructs stimulate expression of FOXP3 and HELIOS when the Treg cells are cultured without exogenous IL-2.

Staining protocol for FACS: Cells were cultured and analyzed as described in Example 1.

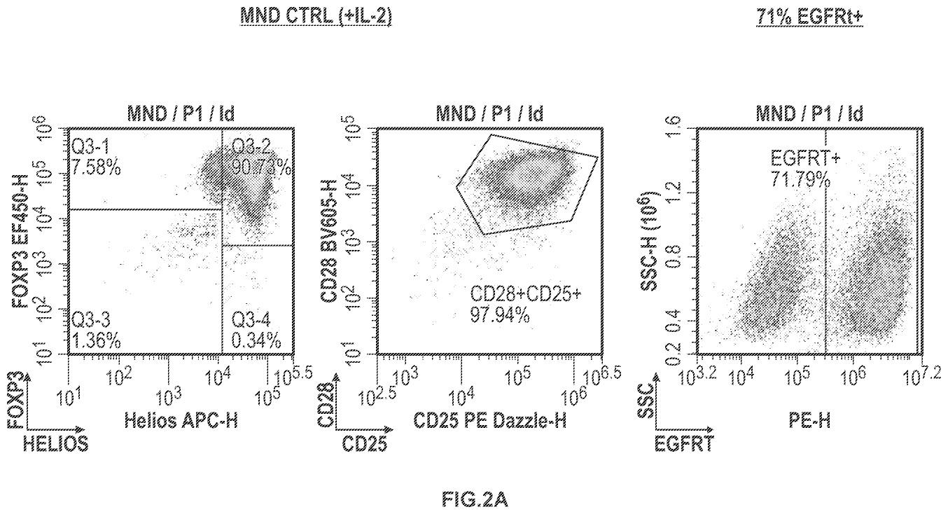

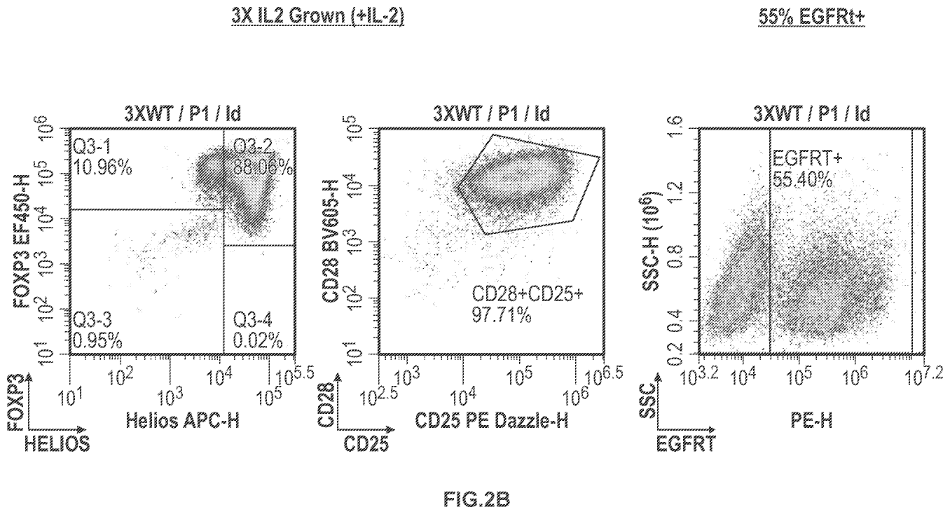

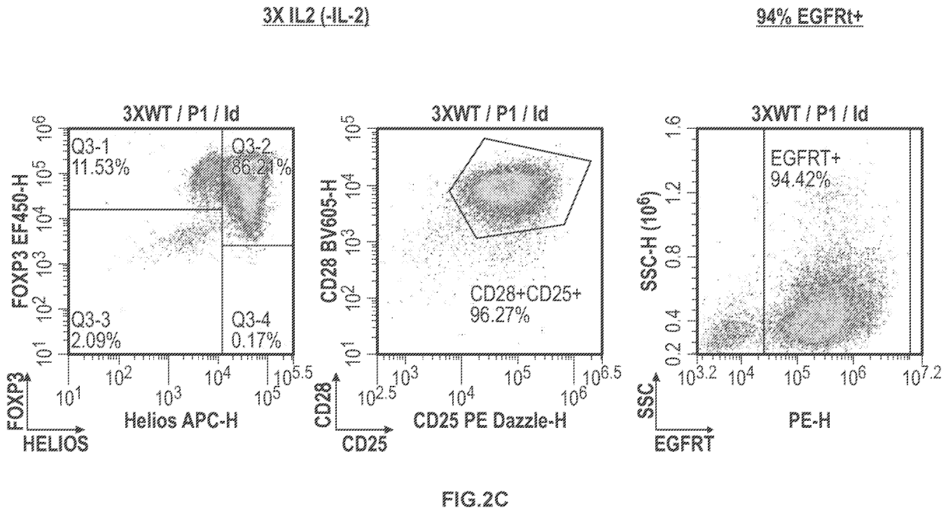

Results: FIGS. 2A-2C depicts the expression of Treg lineage-specific markers by representative FACS plots from each experimental and control condition over time. FIG. 2A shows the expected baseline expression levels of FOXP3, HELIOS and IL-2Rα in IL-2-treated control Tregs at Day 14. FIG. 2B shows the percent population of cells positive for FOXP3, HELIOS, and IL-2Rα for the 3×IL-2/IL-2Rβ recombinant cytokine receptor-expressing Treg cells cultured in the presence of exogenous IL-2 cytokine at Day 14. FIG. 2C shows the percent population of cells positive for FOXP3, HELIOS, and IL-2Rα for the recombinant 3×IL-2/IL-2Rβ receptor-expressing Treg cells cultured in the absence of cytokine. Tregs expressing tethered recombinant cytokine receptors that were cultured with or without exogenous IL-2 displayed similar levels of FOXP3, IL-2Rα and HELIOS as the baseline established by control Tregs cultured with IL-2 at Day 14. Importantly, the Tregs cultured without exogenous IL-2 were approximately ˜94% positive for the transgene marker that was a proxy for tethered cytokine expression.

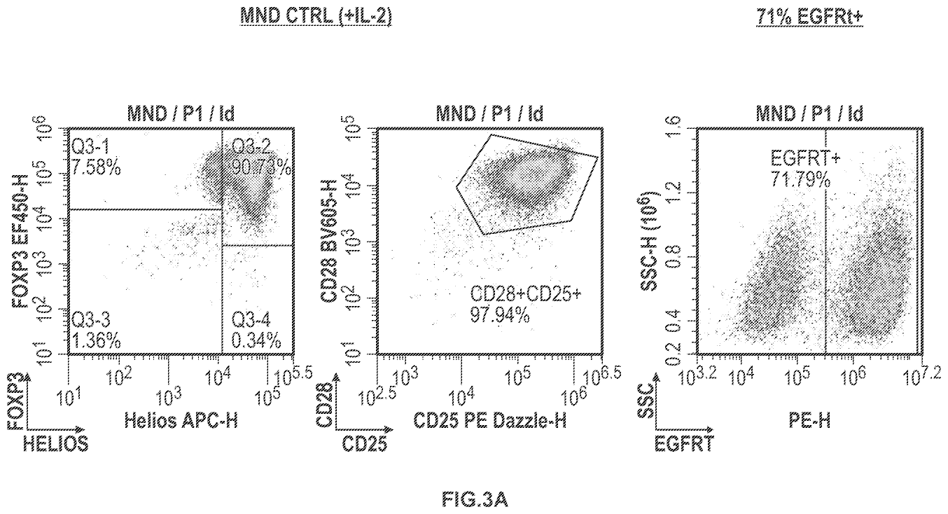

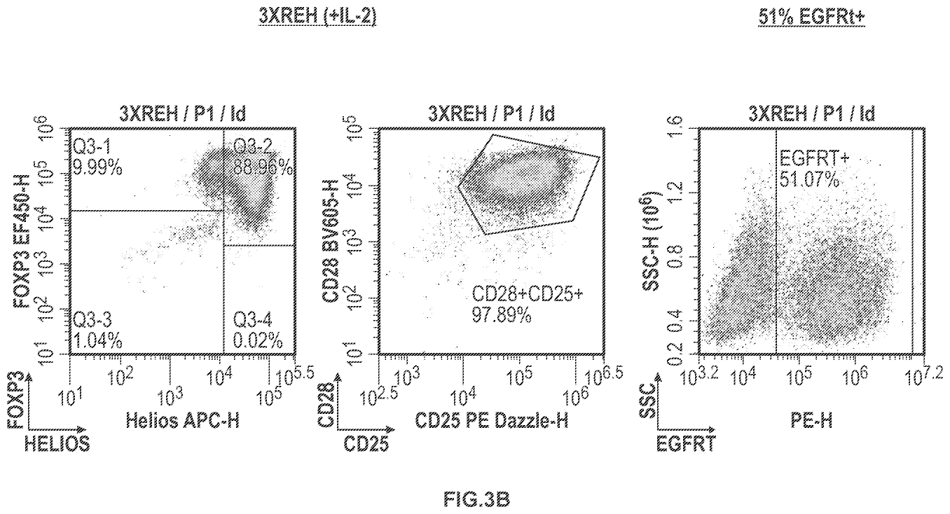

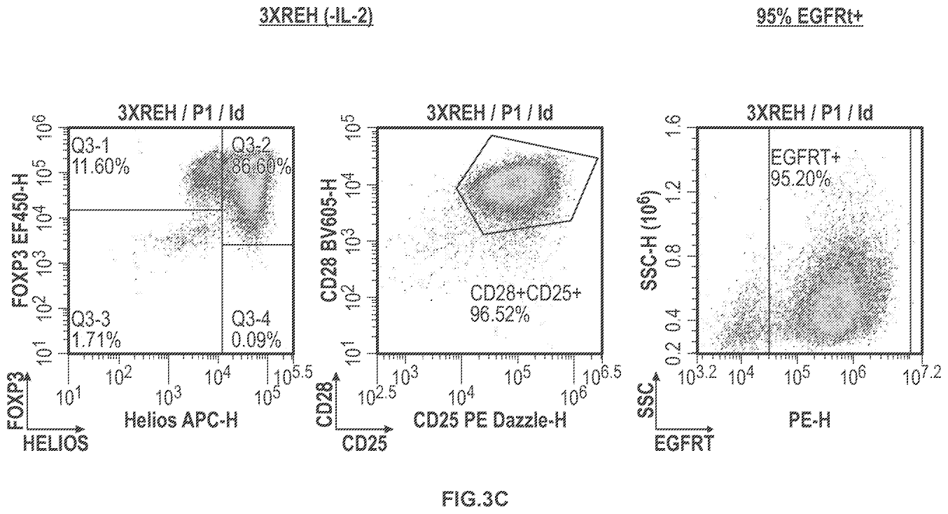

FIGS. 3A-3C depict the expression of Treg lineage-specific markers by representative FACS plots from each experimental and control condition over time. FIG. 3A shows the baseline expression levels of FOXP3, HELIOS and IL-2Rα in IL-2-treated control Tregs at Day 14. FIG. 3B shows the percent population of cells positive for FOXP3, HELIOS, and IL-2Rα for 3×REH IL-2/IL-2Rβ recombinant cytokine receptor-expressing Treg cells cultured in the presence of exogenous IL-2 cytokine at Day 14. FIG. 3C shows the percent of cells positive for FOXP3, HELIOS, and IL-2Rα for the recombinant 3×REH IL-2/IL-2Rβ receptor-expressing Treg cells cultured in the absence of cytokine at Day 14. Tregs expressing tethered recombinant cytokine receptors that were cultured with or without exogenous IL-2 displayed similar levels of FOXP3, IL-2Rα and HELIOS as the control Tregs cultured with IL-2 at Day 14. Importantly, the Tregs cultured without exogenous IL-2 were approximately ˜94% positive for the transgene marker that was a proxy for tethered cytokine expression.

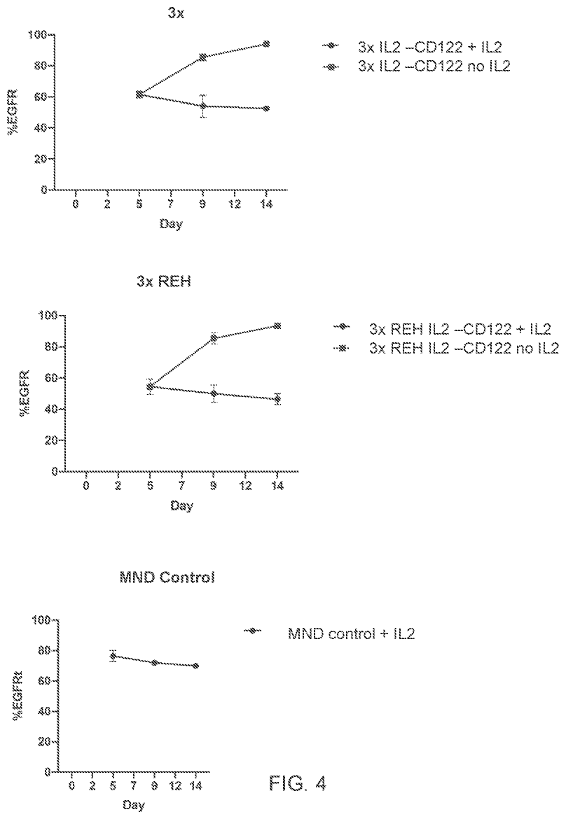

FIG. 4 shows the percent of cells positive for EGFRt days post transduction when grown with or without exogenous IL-2. Average EGFRt percent was calculated from 5 donors. Percent was determined by FACS staining for EGFRt. EGFR+ is a marker for a cell transduced with the construct. Cells expressing the recombinant cytokine receptors cultured in the absence of IL-2 showed similar persistence to control cells that do not express a recombinant cytokine receptor and were cultured in the presence of IL-2 (compare top two panels with bottom panel of FIG. 4).

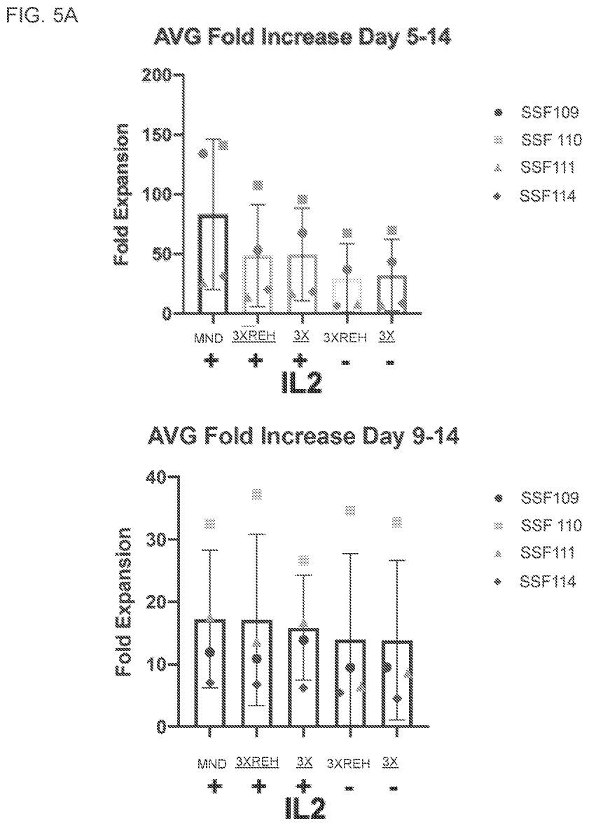

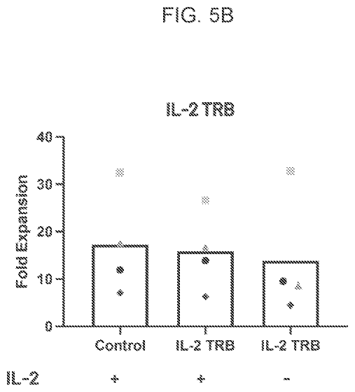

Fold expansion of Tregs expressing the recombinant cytokine receptor grown without cytokine was lower compared to the transduced Tregs grown with IL-2 when measured over the period of 5-14 days. but the fold expansion was similar over the period of 9-14 days (FIG. 5A). FIG. 5B depicts the fold expansion for control and recombinant 3×REH IL-2/IL-2Rβ receptor-expressing Treg cells cultured in the presence or absence of IL-2. Tregs expressing recombinant 3×REH IL-2/IL-2Rβ receptors displayed higher FOXP3 MFI in the absence of IL-2 than, and on par in the presence of IL-2 with, the baseline established by control Tregs cultured with IL-2. These data show that expressing the recombinant cytokine receptor was important for supporting Treg specific cell expansion in the absence of exogenous IL-2, and that the degree of expansion was comparable to Tregs cultured with exogenous IL-2 cytokine.

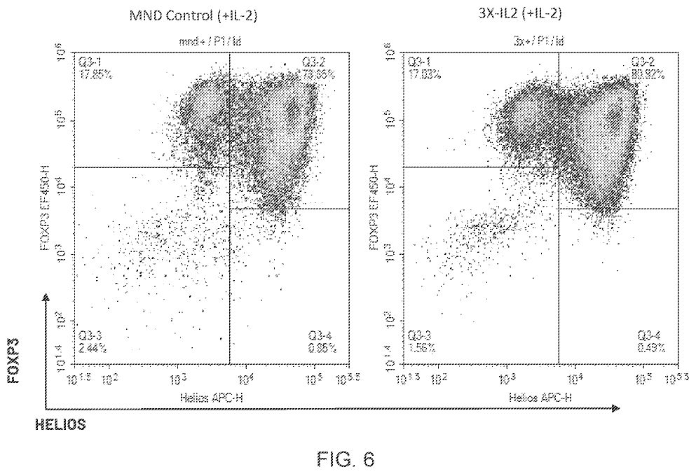

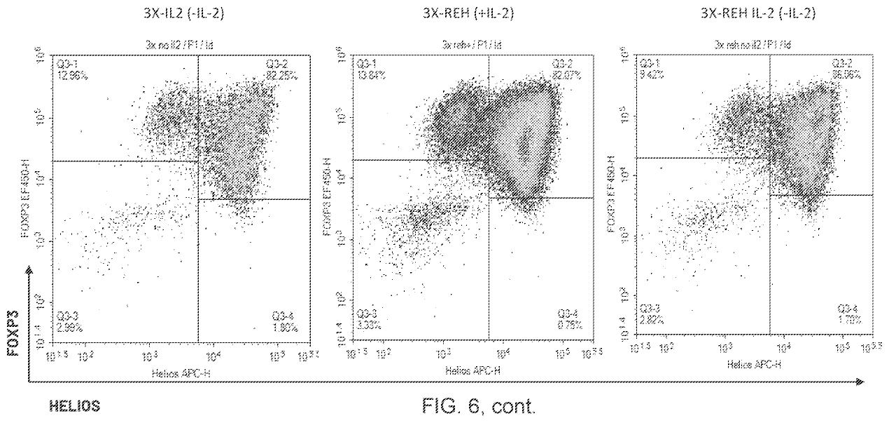

FIG. 6 depicts the expression of Treg lineage-specific markers by representative FACS plots from each experimental and control condition over time. Tregs transduced with empty vector but grown with exogenous IL-2 showed the expected levels of FOXP3 and HELOS for comparison against the Treg profile of 3×REH IL-2/IL-2Rβ recombinant cytokine receptor-expressing Treg grown with and without exogenous IL-2 cytokines at Day 23. Tregs expressing tethered recombinant cytokine receptors that were cultured with or without exogenous IL-2 maintained similar levels of FOXP3, IL-2Rα and HELIOS as the baseline established by control Tregs cultured with IL-2 at Day 23.

### Example 3: Regulatory T Cells Expressing Tethered Recombinant Cytokine Receptors Suppress Effector T Cells

Treg cells limit and suppress effector CD4+ and CD8+ T cell activity to prevent indiscriminate destruction of healthy tissues. Tregs expressing IL-2/IL-2Rβ tethered recombinant cytokine receptors displayed equivalent suppressive behavior towards effector CD4+ and CD8+ T cells without cytokine stimulus as the control Tregs display when cultured with IL-2. The recombinant cytokine receptors are thus able to support and promote Treg suppressive functions in exogenous IL-2 depleted environments par with biologically relevant control Treg suppression levels in exogenous IL-2 rich environments.

Treg suppression assay: Treg cells expressing control vectors or tethered recombinant cytokine receptors were prepared using the same method as described in Example 1 above for 14 days from fresh human leukopaks in a 96-well U-bottom plate. Tregs expressing tethered recombinant IL-2/IL-2Rβ receptors were cultured with or without cytokine, whereas control Tregs expressing an empty vector control were cultured with IL-2. PBMCs were used as Treg responder cells in co-culture with Tregs. PBMCs were isolated as described in Example 1 and then labelled with Cell Trace Violet (CTV) as a cell proliferation dye. PBMCs (5×10⁴/well) were co-cultured with the following Treg effector-to-PBMC target ratios: 1:1, 1:2, 1:4, 1:8, 1:16, and 1:32. The co-culture was activated for 72 hrs using soluble anti-CD3 and anti-CD28 activation heads. The suppressive function of Treg cells was determined by measuring undivided CD4+ and CD8+ T cells after 72 hours.

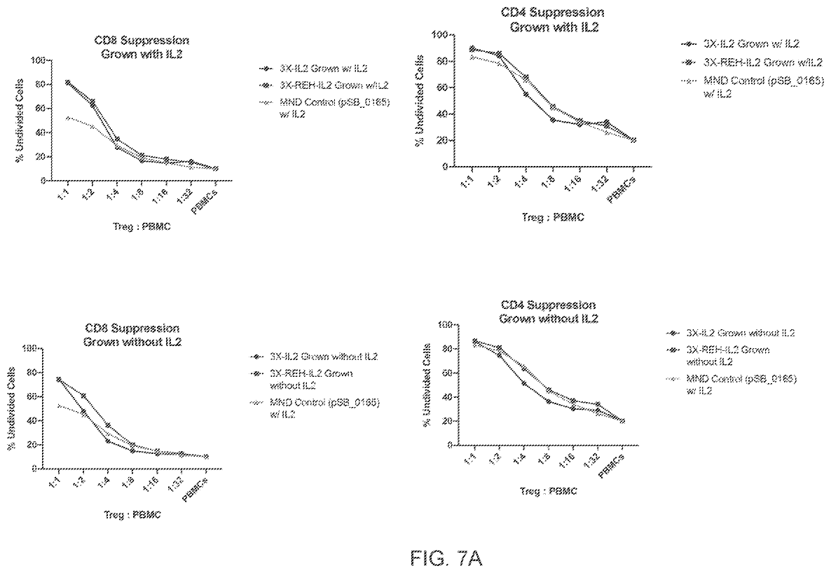

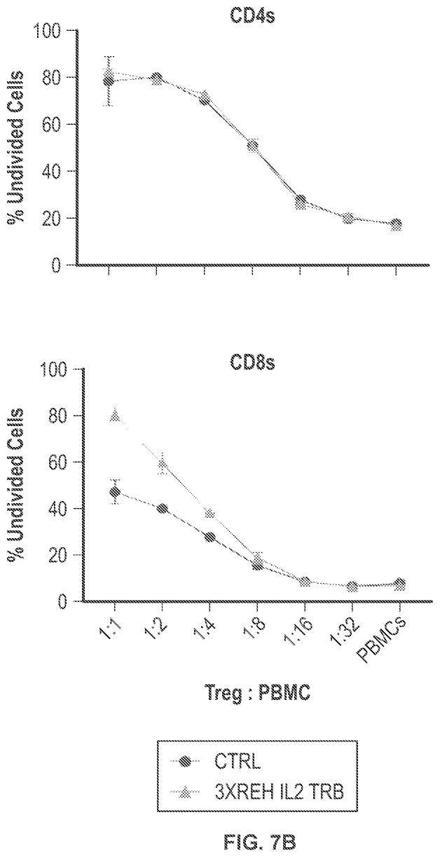

Results: Tregs expressing tethered recombinant cytokine receptor cultured without cytokine were able to suppress CD4+ and CD8+ cell division in a dose dependent manner (FIG. 7A). Transduced Tregs expressing recombinant cytokine receptor grown with or without exogenous IL-2 cytokine suppressed CD4+ and CD8+ T cell division in a ratio-dependent response, such that a higher number of CD4+ or CD8+ T cells to each Treg cell diminished the Treg suppressive impact and resulted in a lower percentage of undivided CD4+ or CD8+ T cells (FIG. 7A). In all charts, the Tregs transduced with empty vector control were grown with exogenous IL-2 and were a positive control. Tregs expressing tethered 3× or 3×REH IL-2/IL-2Rβ recombinant cytokine receptors demonstrated constitutive suppressive action indicative of productive Treg function.

FIG. 7B depicts the suppressive activity of Tregs expressing 3×REH IL-2/IL-2Rβ recombinant cytokine receptors on CD4+ T cells and CD8+ T cells. Tregs expressing 3×REH IL-2/IL-2Rβ recombinant cytokine receptors cultured without cytokine behaved in the same capacity as control Tregs cultured with IL-2: both Treg groups suppressed CD4+ and CD8+ T cell division in a ratio-dependent response. At a one-to-one ratio of Tregs expressing 3×REH IL-2/IL-2Rβ recombinant cytokine receptors and CD4+ or CD8+ T cells, only about 20% of the CD4+ T cells and CD8+ T cells were able to divide.

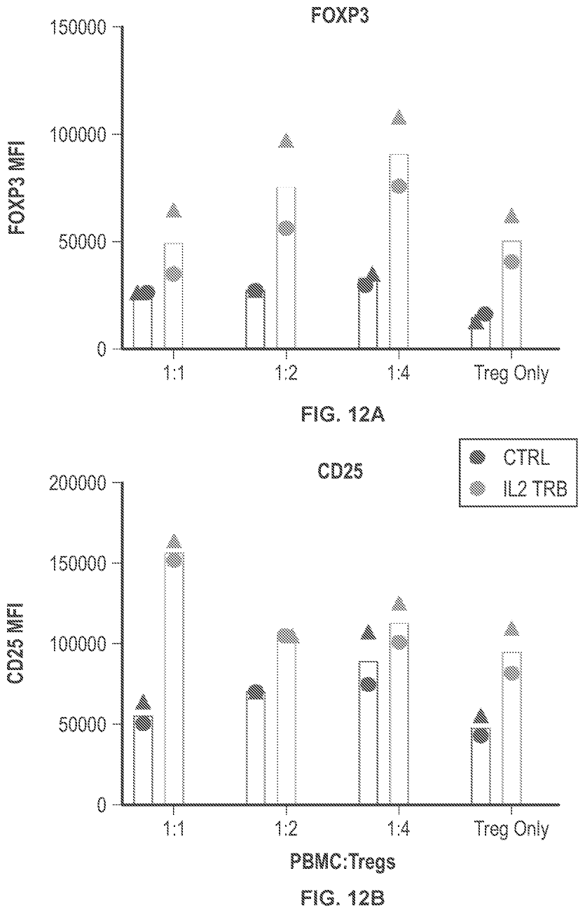

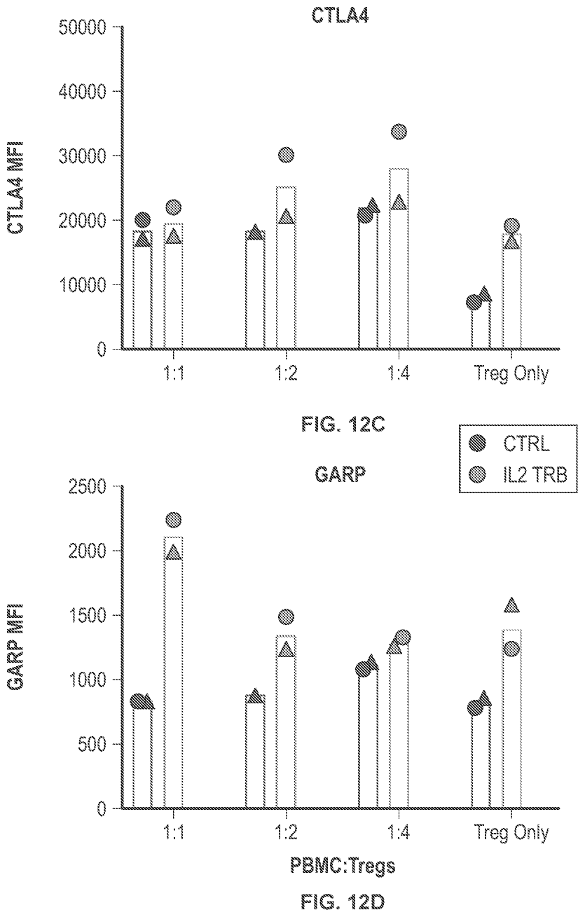

Furthermore, the MFI of FOXP3, CD25, CTLA4, and GARP were analyzed as described above. Tregs expressing 3×REH IL-2/IL-2Rβ recombinant cytokine receptors were cultured alone or co-cultured with PBMCs at a 1:1, 1:2, and 1:4 ratio of PBMCs to Tregs. Cells were then harvested, stained for, and analyzed by flow cytometry, as described above, and the MFI quantified on Treg cells across all four groups. Treg cell levels of FOXP3 (FIG. 12A), CD25 (FIG. 12B), CTLA4 (FIG. 12C), and GARP (FIG. 12D) on co-cultured Tregs expressing 3×REH IL-2/IL-2Rβ recombinant cytokine receptors and PBMCs demonstrated significantly higher levels of each Treg marker (i.e., FOXP3 and CD25) and Treg activation marker (i.e., CTLA4 and GARP) compared to control Tregs at each tested target-to-effector ratio. These results suggest an increase in suppressive function of the Tregs expressing 3×REH IL-2/IL-2Rβ recombinant cytokine receptors over control Tregs.

### Example 4: Tethered Recombinant Cytokine Receptors Induce Constitutive IL-2 Signaling in Regulatory T Cells Across an IL-2 Dose Curve

Tregs expressing 3× and 3×REH IL-2/IL-2Rβ tethered recombinant cytokine receptors exhibited constitutive IL-2 signaling without culturing with exogenous IL-2.

Methods: Treg cells expressing control vectors or tethered recombinant IL-2/IL-2Rβ receptors were prepared using the same method as Example 1 above. Tregs were cultured as described in Example 1. At day 14, Treg cells were pulsed with the following exogenous IL-2 doses: 0.156 U, 2.5 U, or 10 U, then collected and processed for FACS analysis of pSTAT5 as described in Example 2. Representative histograms were overlaid for comparison

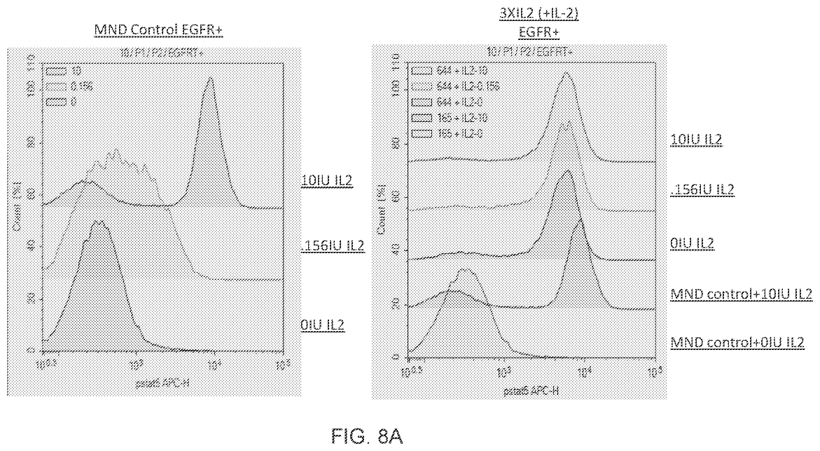

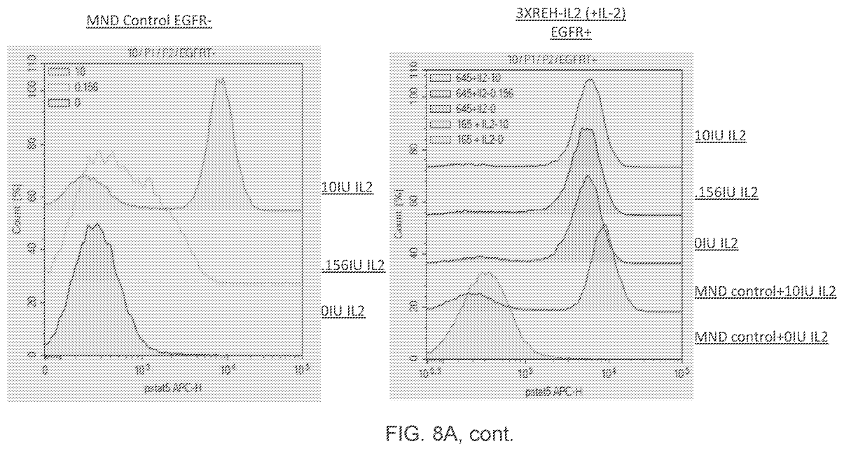

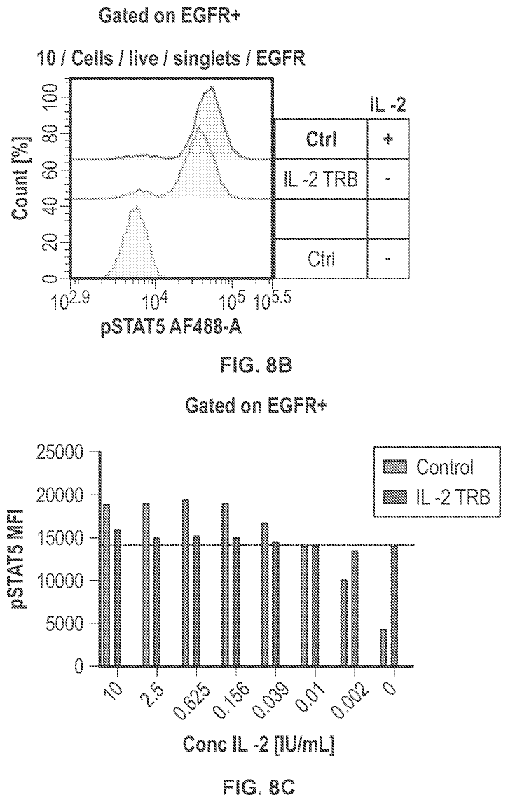

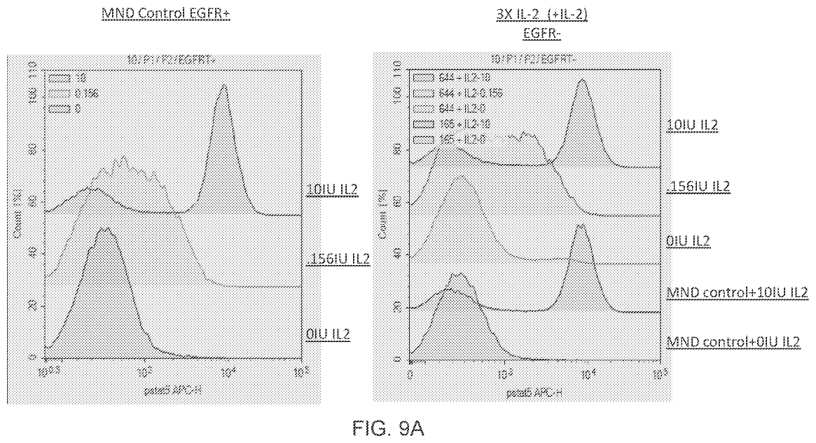

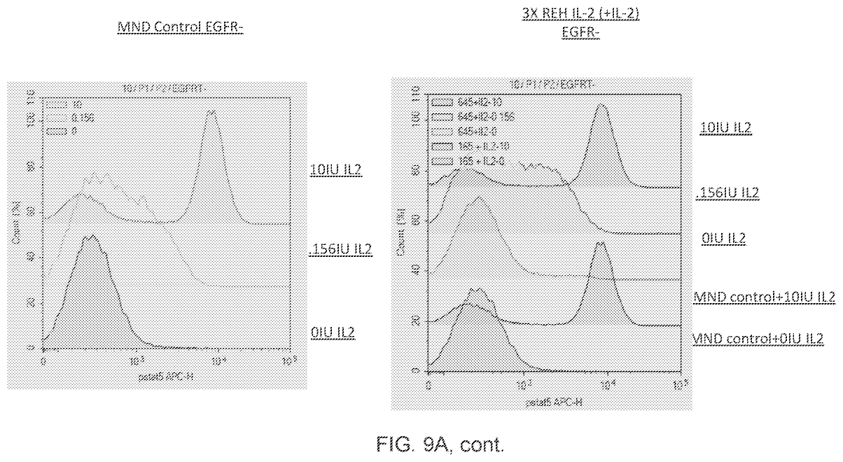

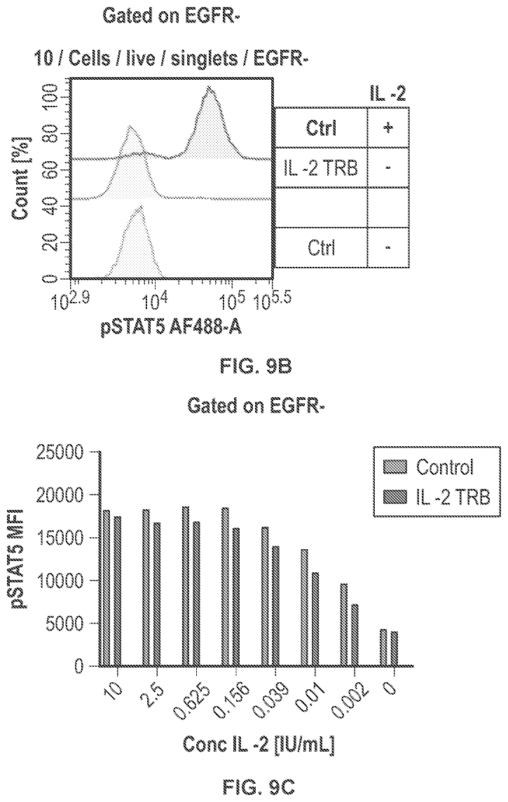

FIGS. 8A-8C depict pSTAT5 signal intensity upon exogenous IL-2 treatment in transduced Tregs expressing empty vector or a mutant IL-2/IL-2Rβ fusion cultured with or without IL-2 treatment. EGFR− cells serve as a negative control to show the degree of pSTAT5 signaling in response to exogenous IL-2 cytokine stimulation. FIGS. 9A-9C depict pSTAT5 signal intensity upon exogenous IL-2 treatment in EGFRt negative (untransduced) Tregs expressing empty vector or a mutant IL-2 cytokine-IL-2Rβ fusion cultured with IL-2 treatment. EGFR− cells served as a negative control to show the degree of pSTAT signaling in response to exogenous IL-2 cytokine stimulation. Across all treatment conditions, pSTAT5 signal intensity in Tregs expressing tethered recombinant IL-2/IL-2Rβ receptors remained constant and high, whereas pSTAT5 signal intensity in control Tregs remained low in the absence of IL-2 and increased upon IL-2 stimulation in a dose-dependent manner. These results demonstrated that the recombinant cytokine receptors induced constitutive IL-2 signaling in Tregs expressing the constructs.

### Example 5: Methylation Analysis

Methods: Tregs were cultured as described in Example 1 and harvested at day 14.

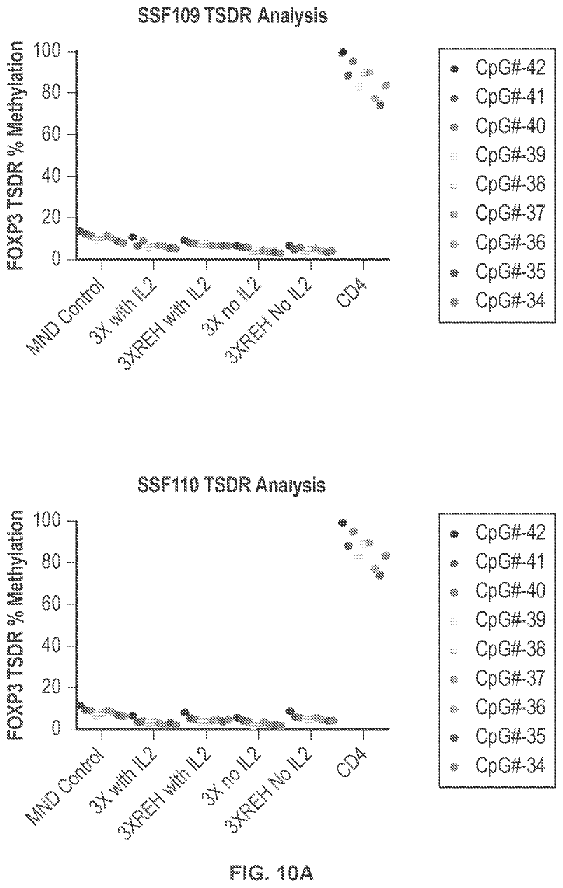

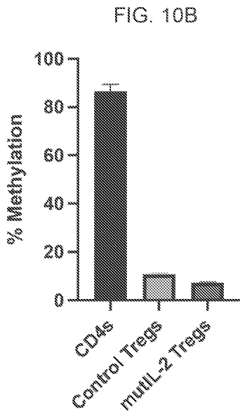

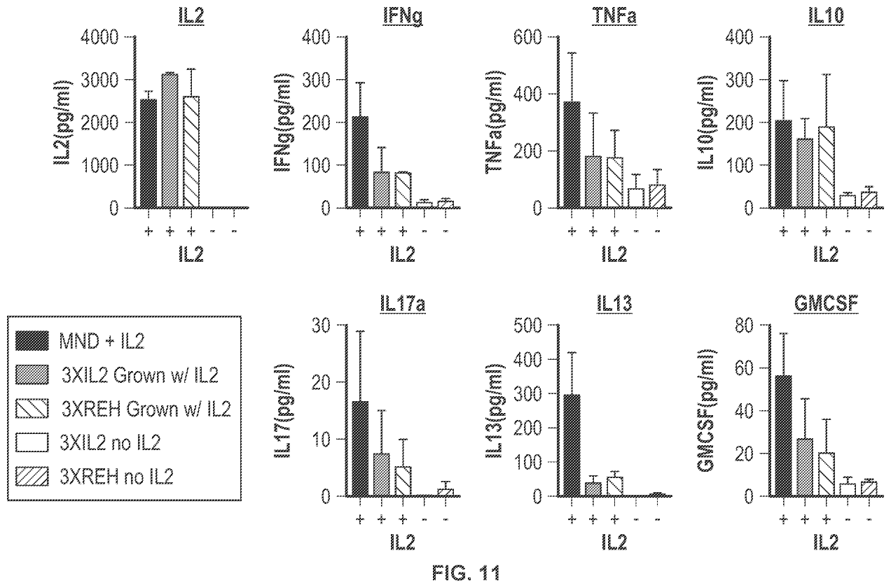

Treg cells expressing empty vector grown with exogenous IL-2 cytokine as a positive control, or Tregs expressing recombinant cytokine receptor grown without exogenous IL-2 cytokine were subjected to methylation analysis at the FOXP3 locus. DNA methylation is a hallmark of gene silencing. In a subset of genes known as Treg specific demethylated region (TSDR), these loci are hypomethylated (e.g., little to no methylation) in Tregs. Therefore, the degree of DNA methylation of CpG sites in TSDR genes is an indicator of gene expression and Treg identity. CpG islands were found to be lowly methylated in transduced Treg cells cultured with exogenous IL-2 cytokine (FIG. 10A). Similarly, the levels of methylation found at FOXP3 CpG islands in Tregs transduced with recombinant cytokine receptor—but not cultured with IL-2—were similarly low, suggesting that the level of FOXP3 expression in transduced samples was equivalent (FIGS. 10A and 10B). As a negative control, CD4+ cells were highly methylated at all FOXP3 CpG sites analyzed (FIGS. 10A and 10B). The data indicate that cells transduced with the IL-2/IL-2 recombinant cytokine receptor have a strong Treg phenotype in the presence or absence of exogenous IL-2 cytokine.

### Example 6: Tregs Expressing Recombinant Cytokine Receptors do not Release Higher Levels of Cytokine

The example below demonstrates that Tregs expressing the recombinant cytokine receptor do not increase their cytokine production when unstimulated.

Methods: Tregs were cultured as described in Example 1 and enriched for EGFRt expression on day 14. Cells were washed, debeaded and rested from days 14-16. IL-2 was added to culture for cells grown in the presence of 300 U/ml IL-2. Cells were re-stimulated on day 16 with heads, and supernatant was collected on Day 19 and cytokine production was quantified by ELISA.

Results: Various cytokines were assayed from the collected cell culture media. For all cytokines analyzed, Tregs expressing the recombinant cytokine receptor did not increase their cytokine production (FIG. 11). Importantly, no IL-2 cleavage was present, which indicated that the lower levels of cytokine release were a property of the Tregs expressing functional 3× and 3×REH IL-2/IL-2Rβ recombinant cytokine receptor. The data suggested that Treg identity could be maintained using the 3× or 3×REH IL-2/IL-2Rβ recombinant cytokine receptor without inducing over-production of additional cytokines without antigenic stimulus.

### Example 7: Regulatory T Cells Expressing Mutein IL-2 Tethered Recombinant Cytokine Receptors Display Trea Profile after Multiple Rounds of Antigen Stimulation

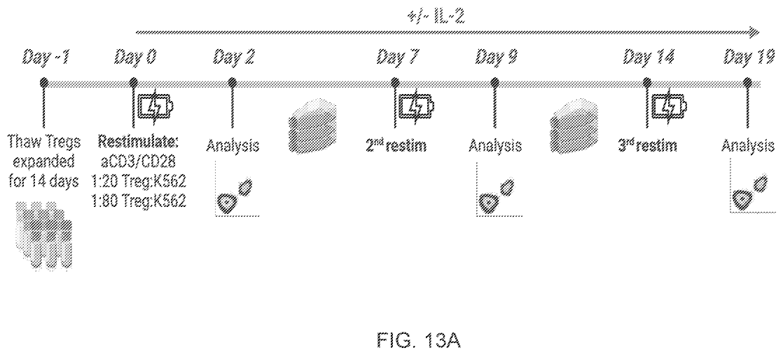

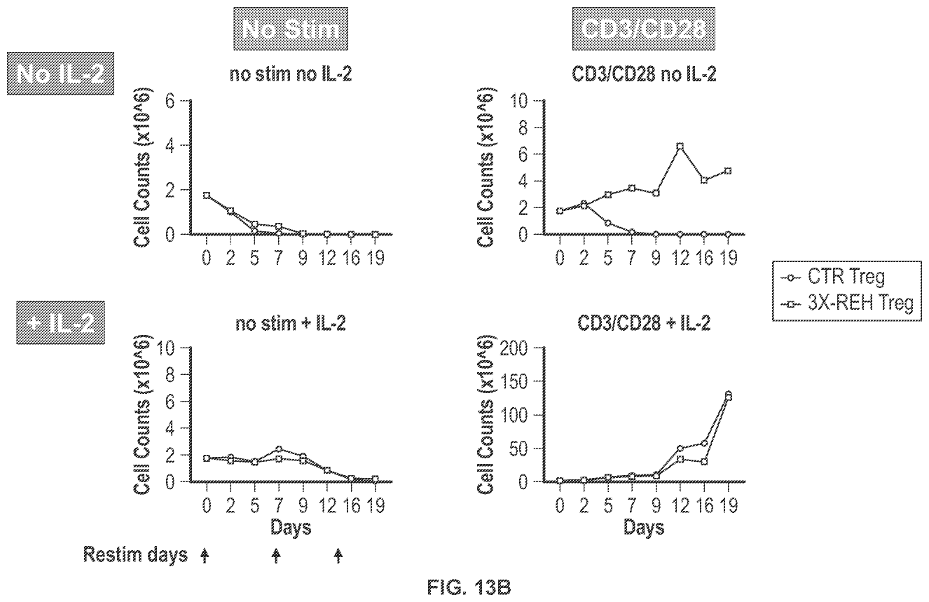

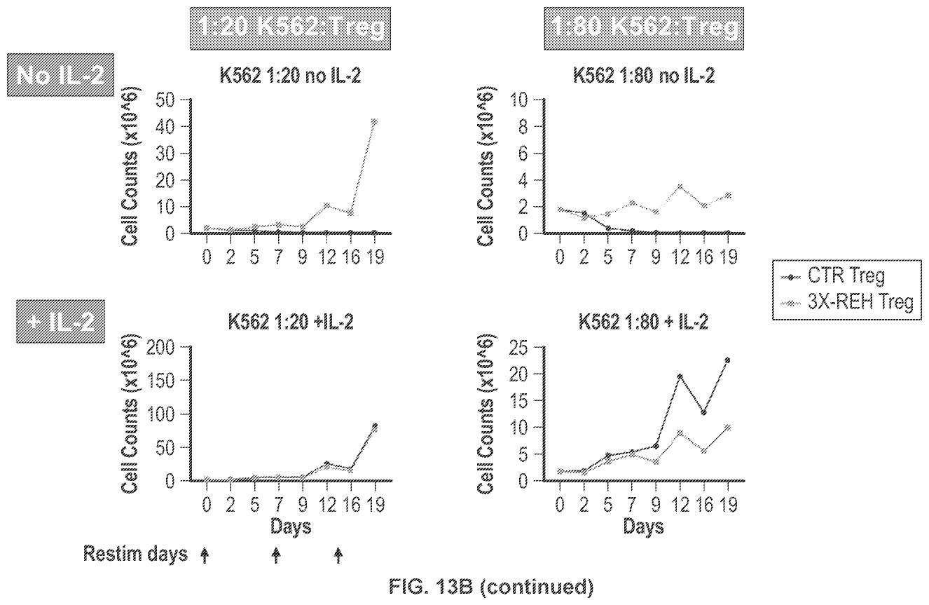

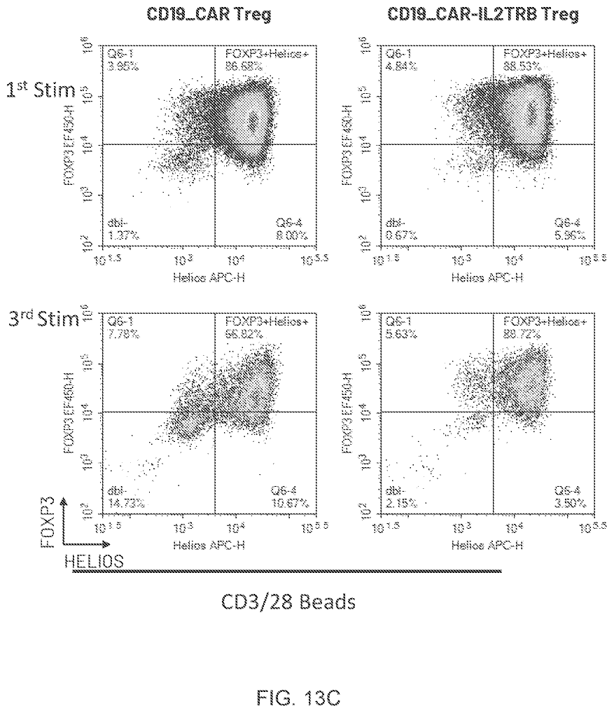

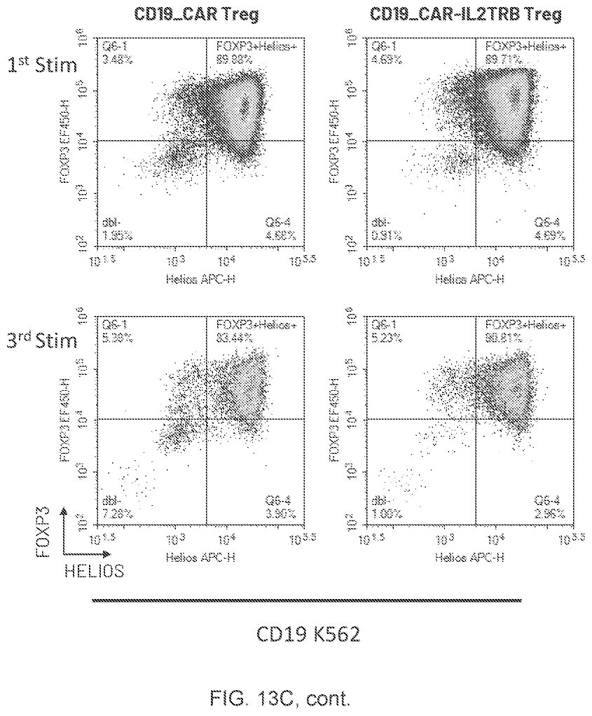

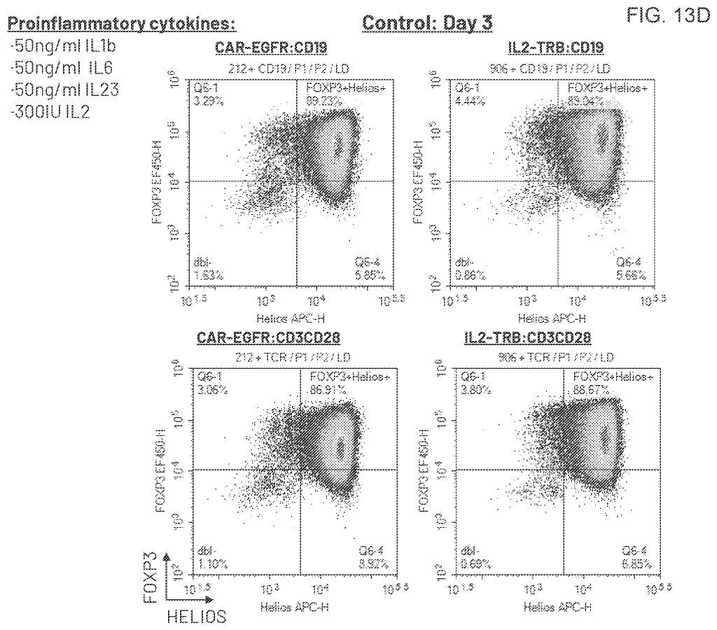

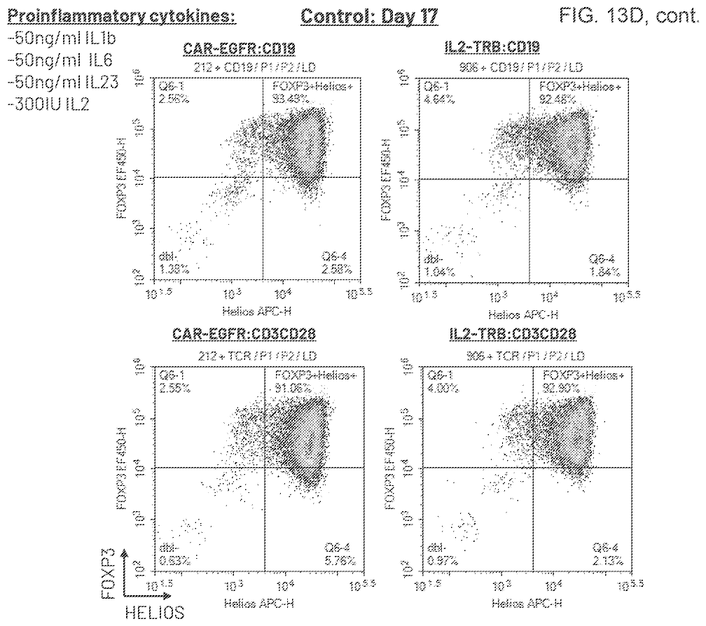

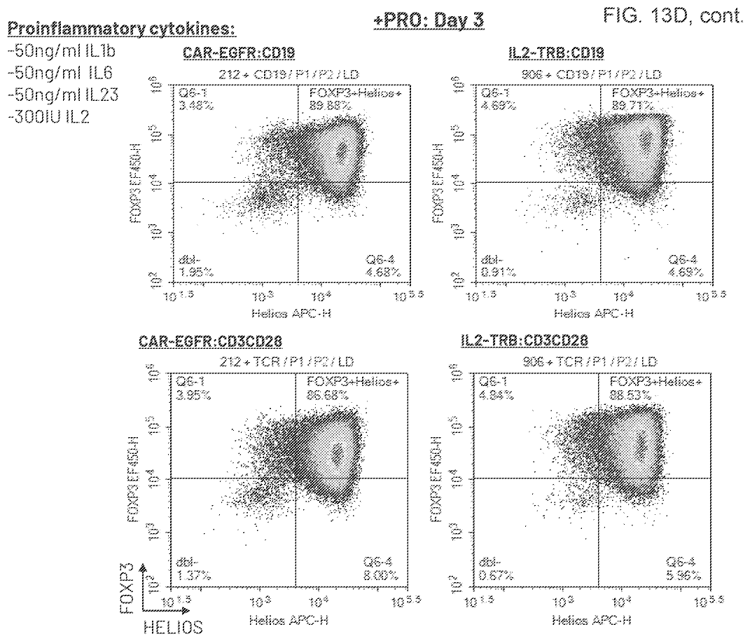

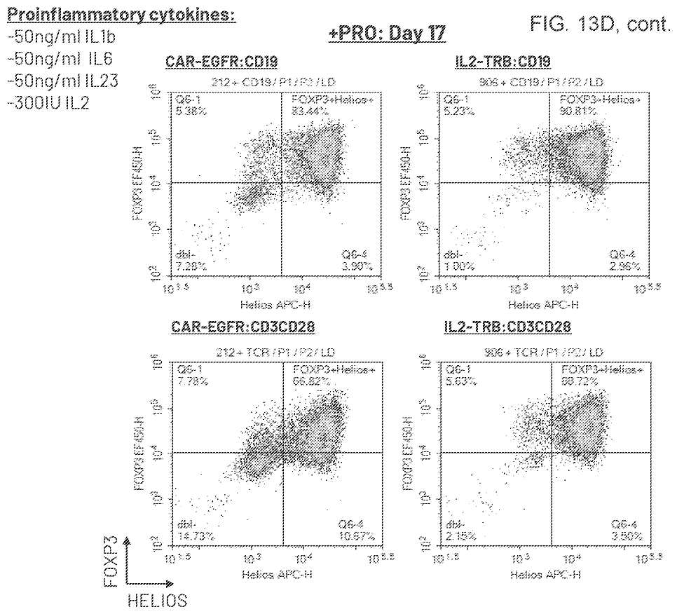

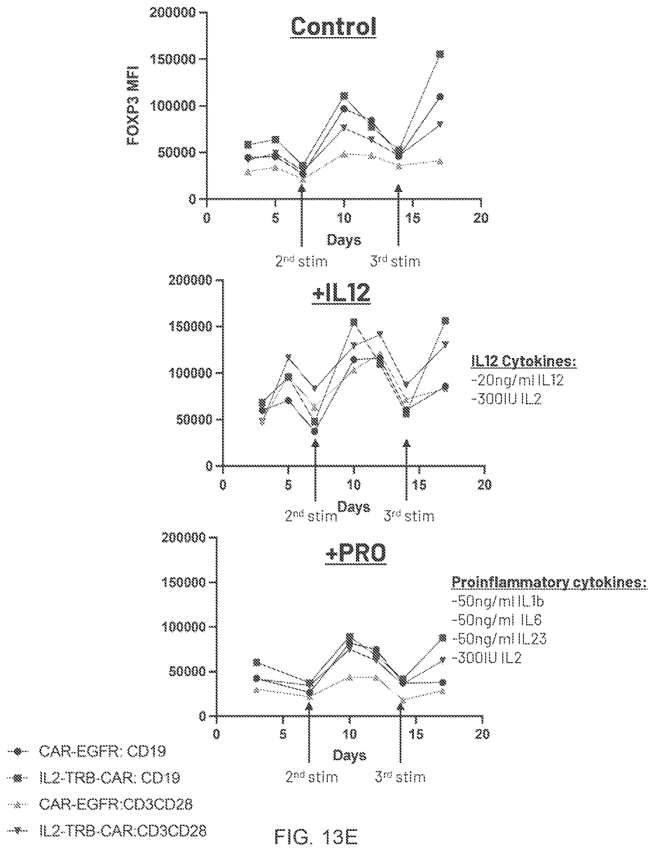

Tregs were cultured in vitro alone, with anti-CD3/anti-CD28 antibody, or co-cultured with CD19-presenting K562 at 1:20 or 1:80 Treg:K562 ratios as shown in FIG. 13A. In brief, Treg cells were thawed and allowed to recover for one day, then activated by anti-CD3/anti-CD28 antibody stimulation or by co-culture with K562 cells that express the CD19 antigen on Day 0. On Day 2, some cells were harvested and analyzed by flow cytometry for Treg phenotype and cytokine production. The remaining cells continued to grow and were re-stimulated on Day 7. Then on Day 9, some cells were harvested and analyzed by flow cytometry for Treg phenotype and cytokine production. The remaining cells continued to grow in vitro, and on Day 14, the Tregs were re-stimulated one file time and the cells harvested for flow cytometric analyses on Day 19.

As shown in FIG. 13B, Tregs expressing 3×REH IL-2/IL-2Rβ recombinant cytokine receptors proliferated after each round of cell stimulation, including both anti-CD3/anti-CD28 antibody stimulation and antigen-specific TCR activation, in both the absence and presence of IL-2. However, control Tregs only proliferated upon combined stimulation conditions and IL-2 treatment. FIG. 13C shows representative FACS plots after first versus third rounds of stimulation by anti-CD3/anti-CD28 antibodies or CD19-antigen-specific activation comparing the FOXP3+/HELIOS+ phenotype in control versus 3×REH IL-2/IL-2Rβ recombinant cytokine receptor-expressing Tregs.

FIG. 13D shows the impact of a pro-inflammatory cytokine milieu on the FOXP3+/HELIOS+ phenotype of control Tregs and Tregs expressing 3×REH IL-2/IL-2Rβ recombinant cytokine receptors. Cells grown in vitro under control conditions and under pro-inflammatory conditions were analyzed at day 3 and day 17 of culture by flow cytometry. Cells that were grown in the pro-inflammatory condition were treated with 50 ng/mL IL-1β, 50 ng/mL IL-6, 50 ng/mL IL-23, and 300 IU IL-2. Cells were further activated by polyclonal anti-CD3/anti-CD28 antibody stimulation or by CD19-antigen stimulation. FIG. 13E shows the FOXP3 mean fluorescence intensity (MFI) overtime for control, IL-12 conditions, and pro-inflammatory conditions. IL-12 conditions involved cell incubation with 20 ng/mL IL-12 and 300 IU IL-2. These results indicate that the Tregs expressing 3×REH IL-2/IL-2Rβ recombinant cytokine receptors show an increase in Treg stability, in particular in a pro-inflammatory environment, and upon CD19-antigen stimulation.

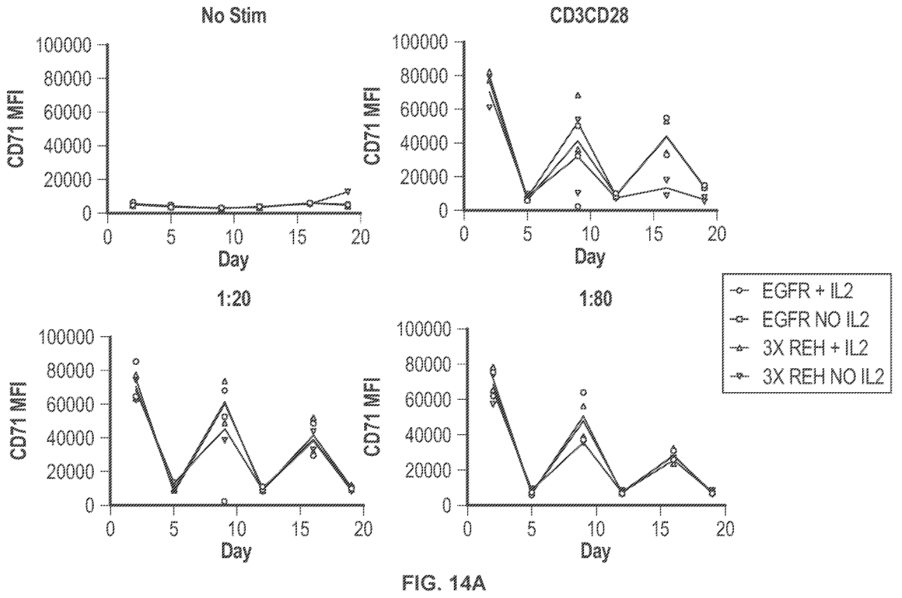

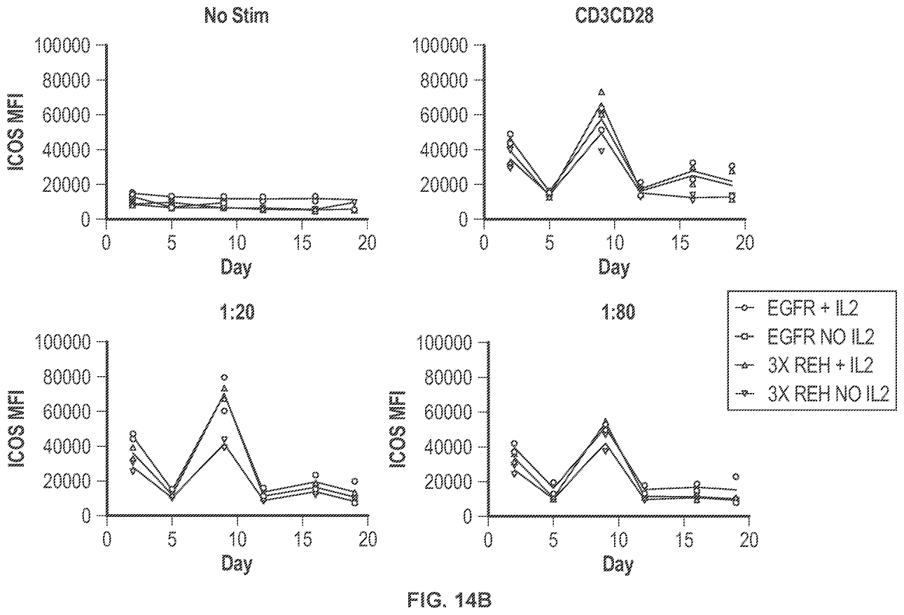

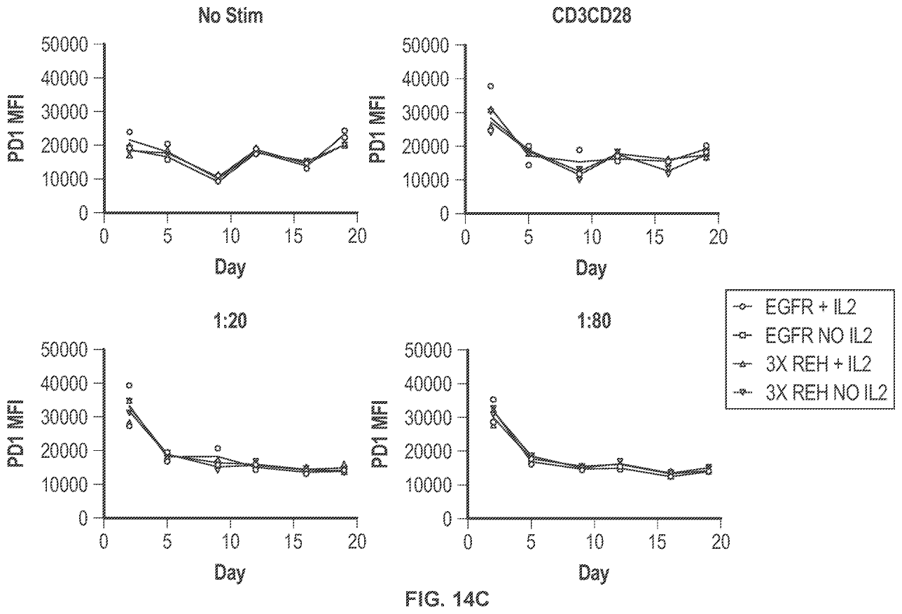

Markers of Tregs, CD71 (FIG. 14A) and ICOS (FIG. 14B), peaked after each round of stimulation but were not present in unstimulated Treg cells. PD-1 levels (FIG. 14C) generally remained low and did not peak or otherwise respond to Treg stimulation. Treg expression of each marker was similar across all conditions, such that there were no observable differences between control Tregs and Tregs expressing 3×REH IL-2/IL-2Rβ recombinant cytokine receptors.

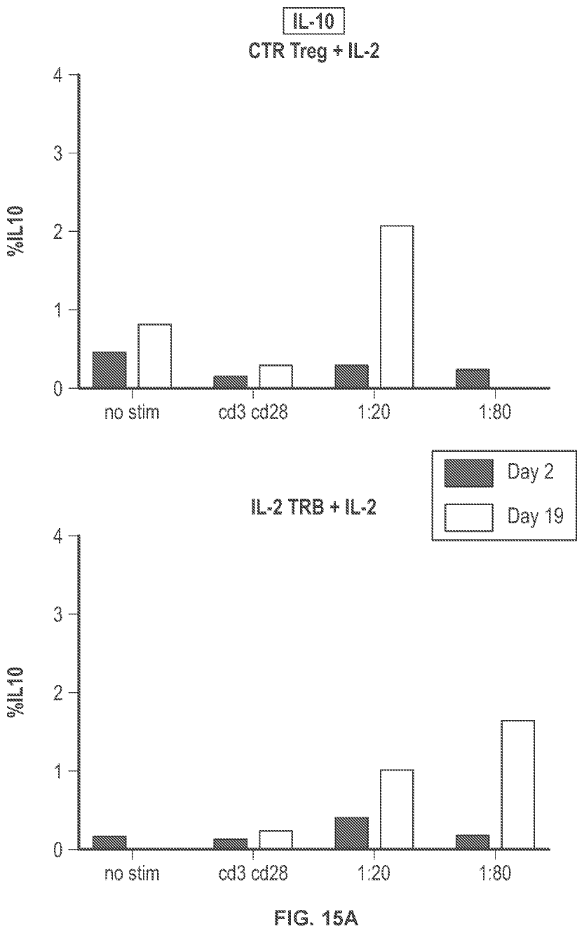

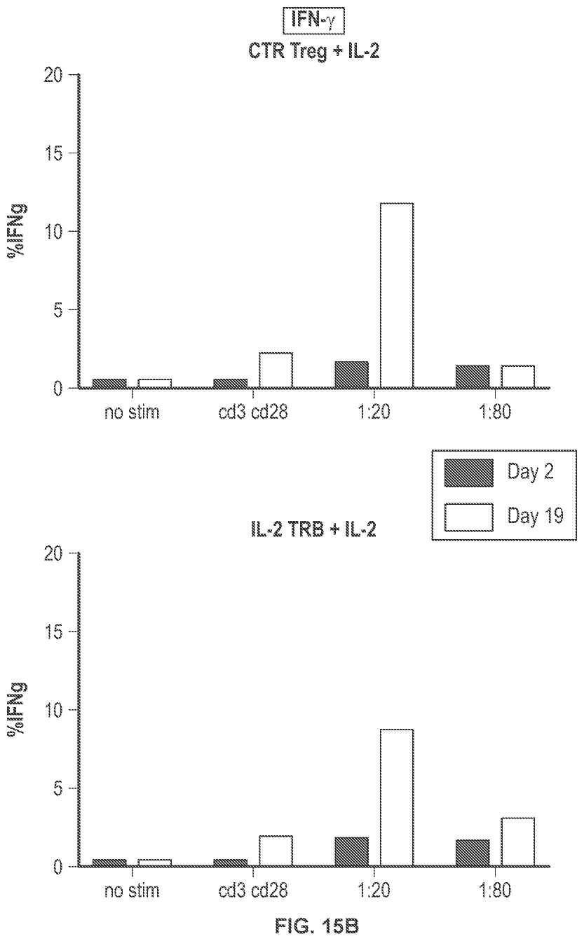

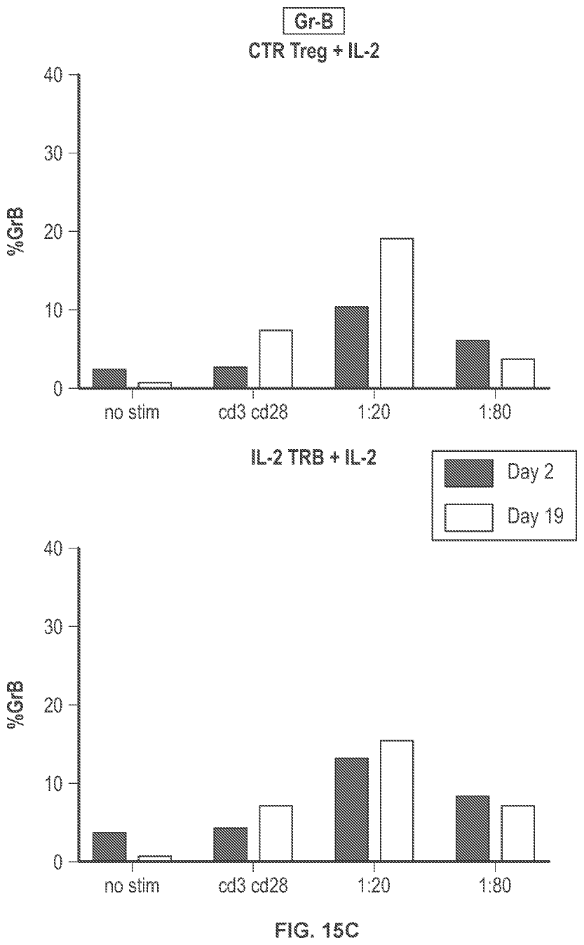

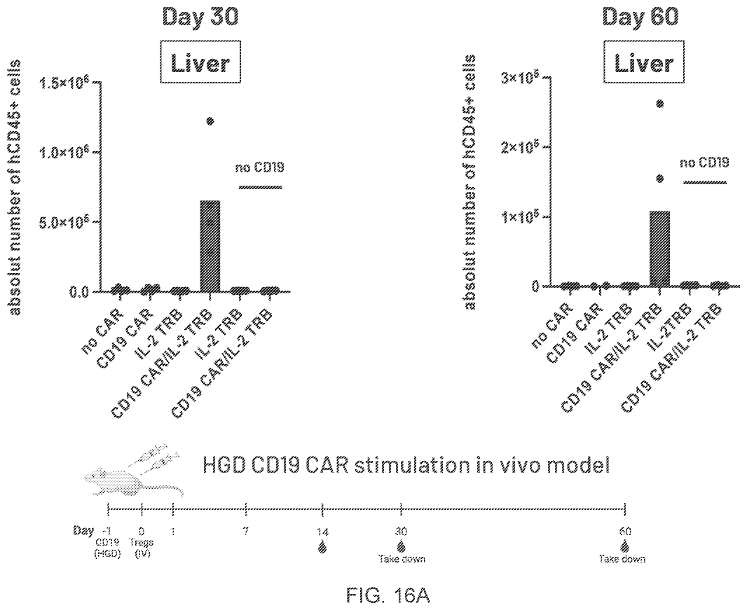

Furthermore, as shown in FIGS. 15A-15C, the production levels of IL-10, IFN-γ, and Granzyme B (GrB), respectively, were elevated in Tregs expressing 3×REH IL-2/IL-2Rβ recombinant cytokine receptors compared to control Treg cells by Day 19 in co-culture with K562-CD19 cells. While control Tregs show a peak in cytokine production after the second round of stimulation, Tregs expressing 3×REH IL-2/IL-2Rβ recombinant cytokine receptors continue to produce cytokines after subsequent rounds of stimulation. These results demonstrate that Tregs expressing 3×REH IL-2/IL-2Rβ recombinant cytokine receptors are able to functionally re-activate to higher levels than control Tregs and therefore can act to suppress effector T cells upon multiple rounds of restimulation.

### Example 8: Engraftment and Expansion of Regulatory T Cells Expressing Mutein IL-2 Tethered Recombinant Cytokine Receptors In Vivo

Immunocompromised mice were injected with CD19 (HOD) or vehicle one day before they were injected with control Tregs or with Tregs expressing 3×REH IL-2/IL-2Rβ recombinant cytokine receptors. The Tregs given to half of the mice given control Tregs or Tregs expressing 3×REH IL-2/IL-2Rβ recombinant cytokine receptors also expressed a CD19-CAR to test antigen-specific Treg activation in vivo. On Day 14, blood was collected, and on Day 30 or on Day 60, mice were euthanized, and bone marrow, lung, spleen, and liver were harvested to assess for the absolute number of human CD45+ immune cells.

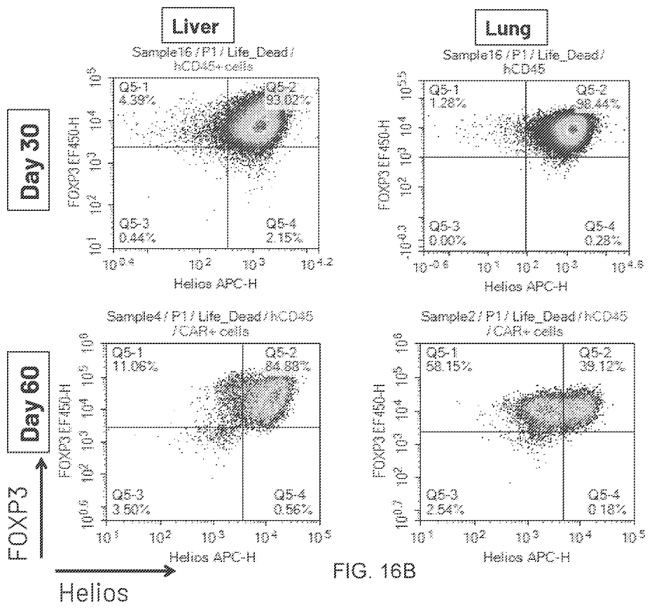

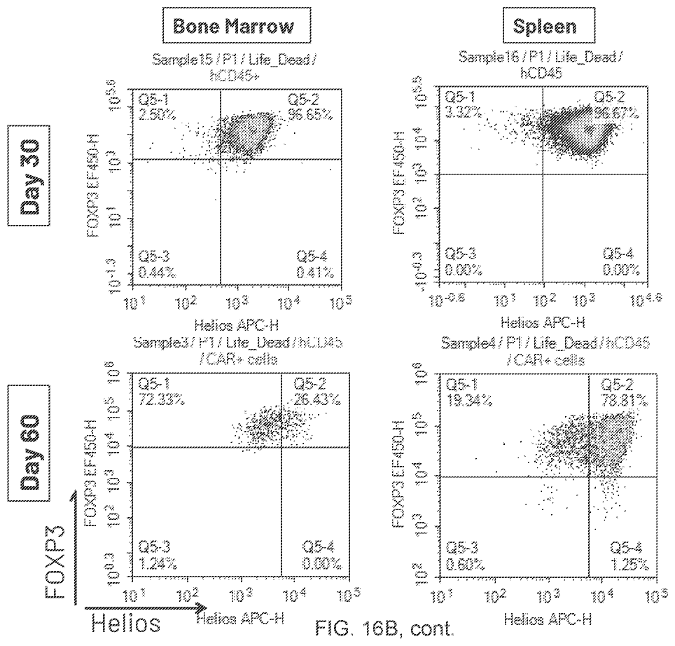

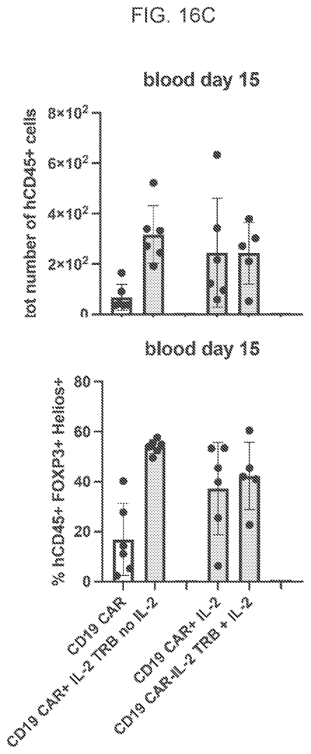

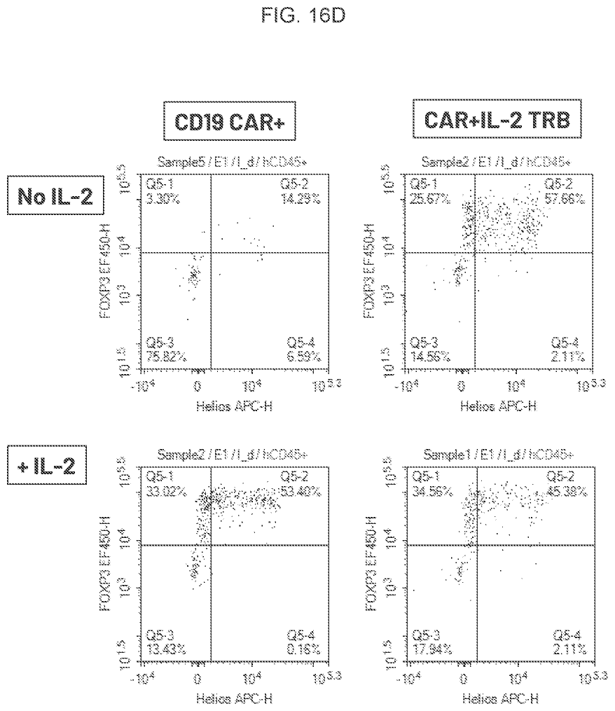

As shown in FIG. 16A, mice injected with CD19-CAR Tregs expressing 3×REH IL-2/IL-2Rβ recombinant cytokine receptors showed significant human immune cell infiltration and expansion compared to mice injected with control Tregs or Tregs expressing 3×REH IL-2/IL-2Rβ recombinant cytokine receptors that lacked the CD19-CAR or were injected in mice not administered CD19. Only Treg cells expressing recombinant cytokine receptors that were CAR-activated showed prolonged Treg proliferation. These results thereby confirm the in vitro data and demonstrate that Treg engraftment and expansion in the absence of exogenous IL-2 in an in vivo murine model requires antigen stimulation to support Treg persistence over time.

As shown in FIG. 16B, across all four tissues, tissues from the mice injected with Tregs expressing 3×REH IL-2/IL-2Rβ recombinant cytokine receptors showed consistent high levels of FOXP3 and HELIOS, thereby confirming the in vitro data and demonstrating Treg engraftment and expansion in the absence of exogenous IL-2 in an in vivo murine model supports long-term Treg persistence and phenotypic profile.

Furthermore, results from FIG. 16C (graphical representation) and FIG. 16D (FACS plots) assessing the total number of human immune cells and percent that were FOXP3+/HELIOS+ Tregs circulating in the blood of the immunocompromised mice at day 15 after intravenous injection, showed that there was a two-fold increase in the total number and percent of Tregs that expressed the 3×REH IL-2/IL-2Rβ recombinant cytokine receptors both with and without IL-2 treatment compared to control Tregs without IL-2 treatment.

### Example 9: Functional Activity of Regulatory T Cells Expressing Mutein IL-2 Tethered Recombinant Cytokine Receptors In Vivo

After confirming the in vivo capacity of the Tregs expressing a recombinant cytokine receptor to engraft, survive, and proliferate, including across various tissues that contain a robust immunological presence, the suppressive activity of the Tregs was analyzed.

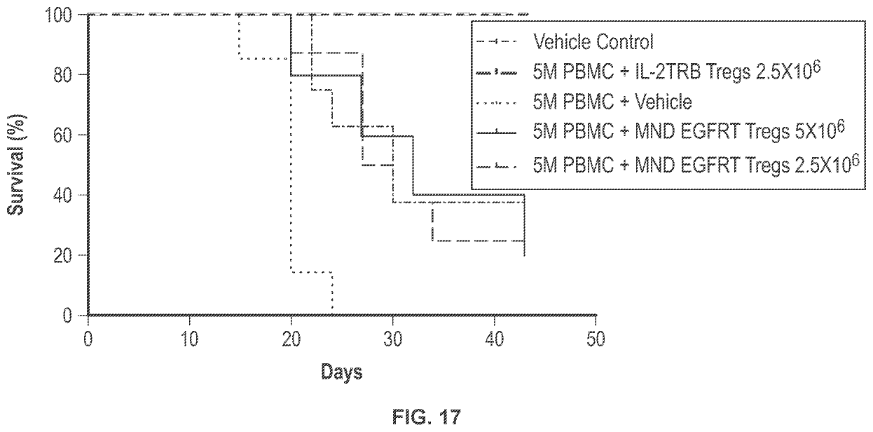

Treg suppressive activity was then tested to compare the ability of control Tregs and Tregs expressing 3×REH IL-2/IL-2Rβ recombinant cytokine receptors to suppress conventional T cells in an immunocompromised mouse model, i.e., to prevent the development of graft versus host disease (GVHD) within the mice. An in vivo model of Treg suppressive activity tested the ability of Tregs expressing 3×REH IL-2/IL-2Rβ recombinant cytokine receptors to suppress conventional T cells in an immunocompromised mouse model such as to prevent the development of graft versus host disease (GVHD) within the mice. In short, NSG mice were intravenously injected with vehicle, 5×10⁶ PBMC and vehicle, 5×10⁶ PBMC and 2.5×10⁶ control Tregs, 5×10⁶ PBMC and 5×10⁶ control Tregs, or 5×10⁶ PBMC and 2.5×10⁶ Tregs expressing 3×REH IL-2/IL-2Rβ recombinant cytokine receptors and then observed over time for survival post-injection. FIG. 17 provides a survival curve of the mice in each group. Mice that were given 5×10⁶ PBMC and 2.5×10⁶ Tregs expressing 3×REH IL-2/IL-2Rβ recombinant cytokine receptors showed a similar survival curve to mice that were given vehicle alone without any PBMCs, showing the functional efficacy of Tregs expressing 3×REH IL-2/IL-2Rβ recombinant cytokine receptors to prevent completely GVHD in vivo.

### Example 10: Alternative IL-2 Independent Strategies

A number of alternative strategies for achieving IL-2 independent Tregs were tested. As further explained below, none of these alternatives produced suitable IL-2 independent Treg cells.

Constitutively active STAT5 Treg model: The activation of STAT5 via its phosphorylation is downstream of IL-2 receptor binding its ligand. As a result, JAK1 and/or JAK3 bind to and phosphorylate STAT5, thereby activating STAT5 for nuclear translocation and binding to its target DNA sequences for gene regulation. In order to test for the efficacy of targeting STAT5 activation status to promote Treg proliferation and downstream function (e.g., effector T cell suppression), a FoxP3^{Cre-ERT2} mouse was crossed to the Rosa^{Stat5bCA} mouse in order to generate the Rosa^{Stat5bCA}FoxP3^{Cre-ERT2} mouse with inducible STAT5 constitutive activity (CA-STAT5 or CA-STAT5b). These mice were then administered a myelin basic protein peptide (MBP) fragment that induces an autoimmune response directed to the myelin sheath surrounding motor neurons as a model of multiple sclerosis (known as experimental autoimmune encephalomyelitis, or EAE). Mice were scored on the standard EAE scale of 1-5 (see Table 2 below) after immunization, up until Day 21.

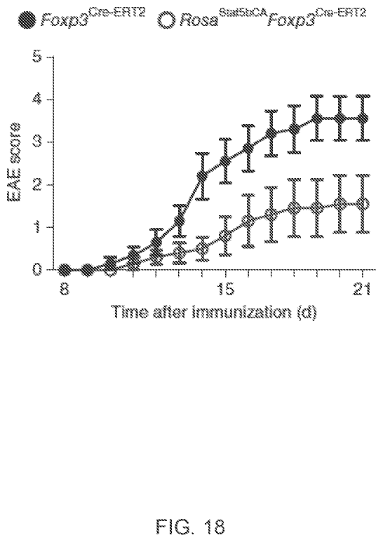

Mice with constitutively active STAT5 showed significant reduction in EAE score, indicating that STAT5 activity was protective against a neurological autoimmune disorder, possibly via its role in Treg activation, as shown in FIG. 18.

TABLE 2
EAE scores.

| Clinical Score | Symptoms |
|---|---|
| 0 | No clinical signs; normal activity |
| 1 | Limp tail or hind limb weakness, but not both |
| 2 | Limp tail and hind limb weakness |
| 3 | Partial hind limb paralysis |
| 4 | Complete hind limb paralysis |
| 5 | Moribund state; death by EAE |

CA-STAT5 Tregs were then harvested from the mice and compared to wildtype (WT) Tregs that were treated or untreated with IL-2 in vitro. Cells were then counted to assess proliferation/expansion, assessed by flow cytometric methods described in the Examples above to analyze the Treg profile, co-cultured with CD4 or CD8 T cells to analyze suppressive activity, or injected into a mouse model of graft versus host disease (GVHD) to test the impact of the CA-STAT5 Tregs on effector T cells in vivo.

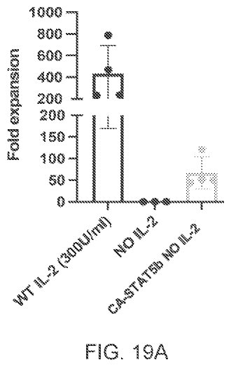

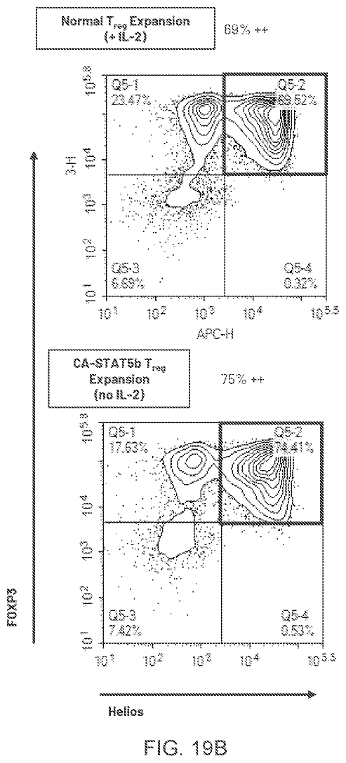

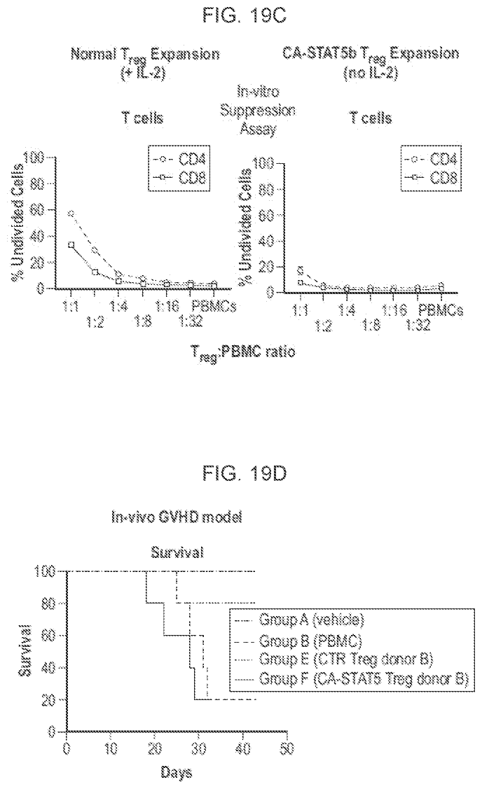

FIG. 19A depicts the expansion of CA-STAT5 Tregs in vitro, albeit at a much-reduced level compared to WT Tregs treated with IL-2. FIG. 19B demonstrates that CA-STAT5 Tregs display a normal Treg phenotype using FOXP3 and HELIOS as Treg markers, both with and without IL-2 treatment. However, FIG. 19C shows a decreased level of CD4 or CD8 T cell suppression in co-culture compared to control Treg cells. This reduced suppressive activity was confirmed in vivo in FIG. 19D, where the mice receiving an injection of CA-STAT5 Tregs showed a lower percent of overall survival compared to control Tregs by 30-40 days.

Furthermore, RNA sequencing was performed on RNA samples that were isolated from wildtype Tregs and CA-STAT5 Tregs to compare gene signatures. Standard bioinformatic analysis was performed on the RNA sequencing results in order to identify any statistically significant differentially expressed genes (DEGs) between the two conditions.

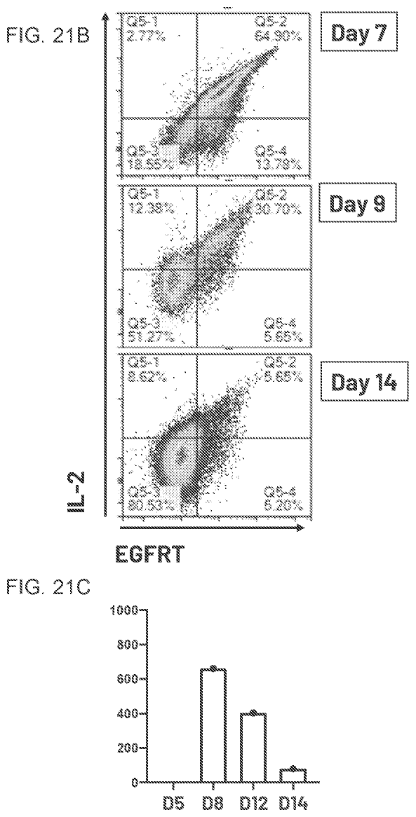

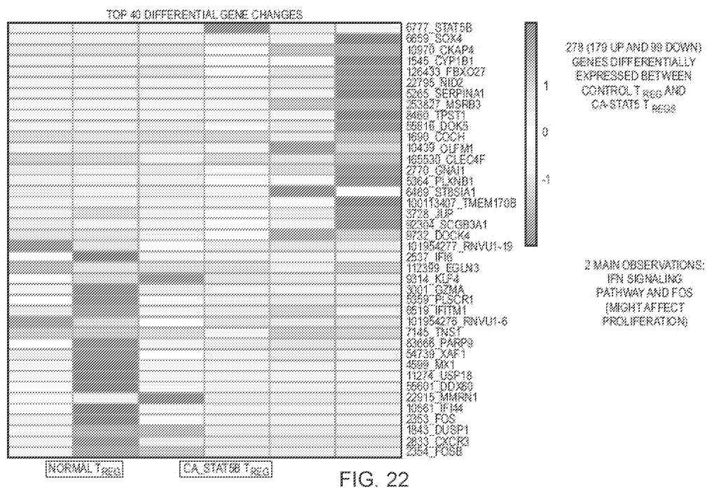

278 DEGs were identified in CA-STAT5 Tregs compared to wildtype Tregs, including 99 downregulated and 179 upregulated DEGS in CA-STAT5 Tregs compared to wildtype Tregs. FIG. 22 shows a heatmap of the top 40 DEGs analyzed by RNA sequencing analysis. Of these DEGS, the main pathways identified from these analyses include interferon pathway family members and FOS pathway family members.

The results from this model demonstrates that the CA-STAT5 cells expanded in the absence of IL-2, albeit not to the same level as wild-type cells treated with IL-2, and the CA-STAT5 cells exhibited a lower level of CD4 and CD8 T cell suppression compared to control Tregs. Accordingly, this model demonstrates that the results presented in FIGS. 1A-17 and Examples 1-9 above are unique and surprising.

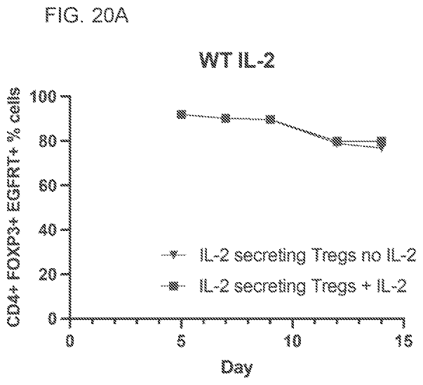

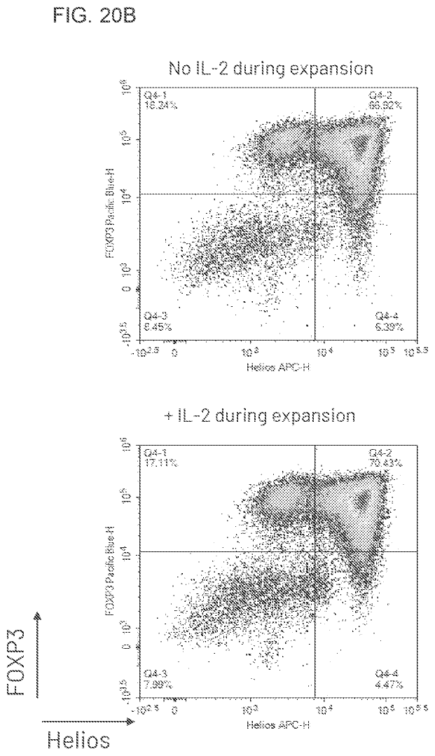

IL-2 secreting Treg model: Tregs were generated that produced and secreted IL-2 to provide IL-2 in an autocrine loop with the goal of self-supporting Treg proliferation. However, when tested in vitro. IL-2 secreting Tregs showed limited expansion (Data not shown). FIG. 20A shows that the IL-2 secreting Tregs display the same percentage of IL-2 secreting Tregs over time when cultured with versus without exogenous IL-2, including the same 10% decrease in Tregs over time. FIG. 20B demonstrates that IL-2 secreting Tregs display a normal Treg phenotype using FOXP3 and HELIOS as Treg markers, both with and without IL-2 treatment. However, in a co-culture experiment with PBMCs, the IL-2 secreting Tregs failed to suppress effector T cells, similarly to the CA-STAT5 Tregs (Data not shown). In line with the CA-STAT5 Treg model, this model also demonstrates that the results presented in FIGS. 1A-17 and Examples 1-9 above are unique and surprising.

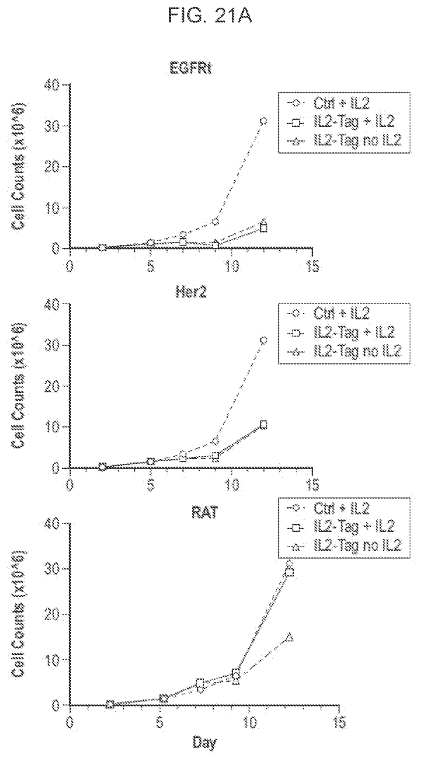

IL-2/Protein-tag tethered Treg model: Tregs were generated where IL-2 was tethered to a protein tag, such as EGFRt, Her2, or IL5Rα tags. This construction method allowed IL-2 to be anchored to the cell membrane to recruit the IL-2 receptor complex and activate the receptor pathway, including STAT5 activation. These Tregs were then tested in vitro for proliferation via cell count (FIG. 21A), Treg persistence over time (FIG. 21B), and IL-2 production (FIG. 21C). As is shown in FIG. 21A, IL-2 tethered Tregs, particularly Tregs where IL-2 was tethered to the EGFRt or Her2 tags, displayed limited expansion over time in the absence of IL-2. Furthermore, these IL-2 tethered Tregs showed a lack of persistence compared to wildtype control Tregs over time, as shown by FIG. 21B. Upon assessing the level of soluble IL-2 in the Treg media over time, it was found that the protein tag (e.g., the Her2 tag tethered to IL-2 as shown in FIG. 21C) released its IL-2 as soluble IL-2 into the media between Days 8-12, thereby reducing the bioavailability of IL-2 directly at the Treg membrane to activate the Tregs long term. As with the CA-STAT5 and IL-2 secreting Treg models described above, this model also demonstrates that the results presented in FIGS. 1A-17 and Examples 1-9 above are unique and surprising.

### Example 11: Gene Expression Analysis

RNA sequencing was performed on RNA samples that were isolated from wildtype Tregs and Tregs expressing 3×/REH IL-2/IL-2Rβ recombinant cytokine receptors, both of which were cultured with IL-2 by standard methods in the field. Standard bioinformatic analysis was performed on the RNA sequencing results in order to identify any statistically significant differentially expressed genes (DEGs) between the two conditions.

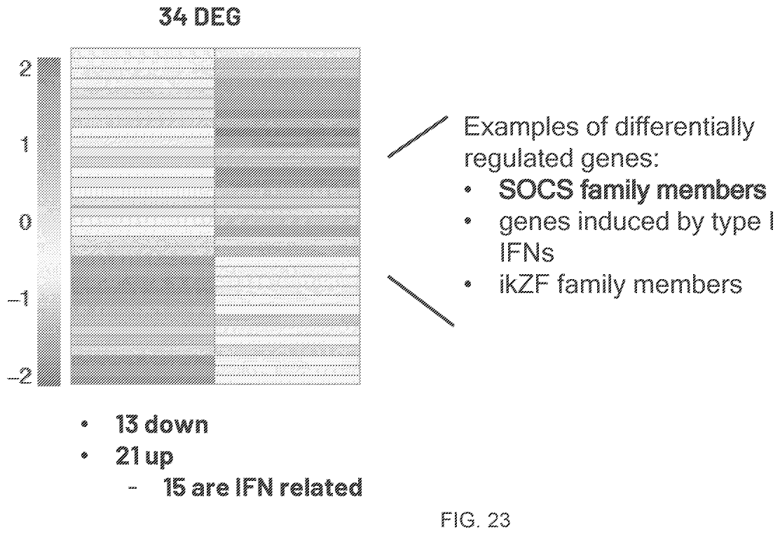

FIG. 23 shows a heatmap of the RNA sequencing analysis, wherein 34 DEGs were identified. Of these DEGS, 13 were downregulated and 21 were upregulated in the Tregs expressing 3×/REH IL-2/IL-2Rβ recombinant cytokine receptors compared to wildtype Tregs, including 15 genes identified belonging to or otherwise related to the interferon pathway. Additionally, DEGs include SOCS family members and ikZF family members.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

SEQ ID NO: 1 (IL-2 tethered IL-2Rβ, human)
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCQSIISTLTEQKLISEEDLEMETSQFPGEEKPQASPEGRPESETSCAVNGTSQFTCFY
NSRANISCVWSQDGALQDTSCQVHAWPDRRRWNQTCELLPVSQASWACNLILGAPDS
QKLTTVDIVTLRVLCREGVRWRVMAIQDFKPFENLRLMAPISLQVVHVETHRCNISWEI
SQASHYFERHLEFEARTLSPGHTWEEAPLLTLKQKQEWICLETLTPDTQYEFQVRVKPL
QGEFTTWSPWSQPLAFRTKPAALGKDTIPWLGHLLVGLSGAFGFIILVYLLINCRNTGP
WLKKVLKCNTPDPSKFFSQLSSEHGGDVQKWLSSPFPSSSFSPGGLAPEISPLEVLERDK
VTQLLLQQDKVPEPASLSSNHSLTSCFTNQGYFFFHLPDALEIEACQVYFTYDPYSEEDP
DEGVAGAPTGSSPQPLQPLSGEDDAYCTFPSRDDLLLFSPSLLGGPSPPSTAPGGSGAGE
ERMPPSLQERVPRDWDPQPLGPPTPGVPDLVDFQPPPELVLREAGEEVPDAGPREGVSF
PWSRPPGQGEFRALNARLPLNTDAYLSLQELQGQDPTHLV
SEQ ID NO: 2 (3x IL-2 tethered IL-2Rβ, human)
APTSSSTKKTQLQLEHLLLDLQMILNGINNYKNPKLTDMLTFEFYMPKKATELKHLQCL
ERELKPLEEVLNLAQSKNFHLRPRDLISNINVIVLELKGSETTFMCEYADETATIVEFLNR
WITFCQSIISTLTEQKLISEEDLEMETSQFPGEEKPQASPEGRPESETSCAVNGTSQFTCFY
NSRANISCVWSQDGALQDTSCQVHAWPDRRRWNQTCELLPVSQASWACNLILGAPDS
QKLTTVDIVTLRVLCREGVRWRVMAIQDFKPFENLRLMAPISLQVVHVETHRCNISWEI
SQASHYFERHLEFEARTLSPGHTWEEAPLLTLKQKQEWICLETLTPDTQYEFQVRVKPL
QGEFTTWSPWSQPLAFRTKPAALGKDTIPWLGHLLVGLSGAFGFIILVYLLINCRNTGP
WLKKVLKCNTPDPSKFFSQLSSEHGGDVQKWLSSPFPSSSFSPGGLAPEISPLEVLERDK
VTQLLLQQDKVPEPASLSSNHSLTSCFTNQGYFFFHLPDALEIEACQVYFTYDPYSEEDP
DEGVAGAPTGSSPQPLQPLSGEDDAYCTFPSRDDLLLFSPSLLGGPSPPSTAPGGSGAGE
ERMPPSLQERVPRDWDPQPLGPPTPGVPDLVDFQPPPELVLREAGEEVPDAGPREGVSF
PWSRPPGQGEFRALNARLPLNTDAYLSLQELQGQDPTHLV
SEQ ID NO: 3 (REH IL-2 tethered IL-2Rβ, human)
APTSSSTKKTQLQLEHLRLDLEMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCHSIISTLTEQKLISEEDLEMETSQFPGEEKPQASPEGRPESETSCAVNGTSQFTCFY
NSRANISCVWSQDGALQDTSCQVHAWPDRRRWNQTCELLPVSQASWACNLILGAPDS
QKLTTVDIVTLRVLCREGVRWRVMAIQDFKPFENLRLMAPISLQVVHVETHRCNISWEI
SQASHYFERHLEFEARTLSPGHTWEEAPLLTLKQKQEWICLETLTPDTQYEFQVRVKPL
QGEFTTWSPWSQPLAFRTKPAALGKDTIPWLGHLLVGLSGAFGFIILVYLLINCRNTGP
WLKKVLKCNTPDPSKFFSQLSSEHGGDVQKWLSSPFPSSSFSPGGLAPEISPLEVLERDK
VTQLLLQQDKVPEPASLSSNHSLTSCFTNQGYFFFHLPDALEIEACQVYFTYDPYSEEDP
DEGVAGAPTGSSPQPLQPLSGEDDAYCTFPSRDDLLLESPSLLGGPSPPSTAPGGSGAGE
ERMPPSLQERVPRDWDPQPLGPPTPGVPDLVDFQPPPELVLREAGEEVPDAGPREGVSF
PWSRPPGQGEFRALNARLPLNTDAYLSLQELQGQDPTHLV
SEQ ID NO: 4 (IL-2 cytokine, human)
APTSSSTKKTQLQLEHLLLDLQMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCQSIISTLT
SEQ ID NO: 5 (3x mutated IL-2 cytokine, human)
APTSSSTKKTQLQLEHLLLDLQMILNGINNYKNPKLTDMLTFEFYMPKKATELKHLQCL
ERELKPLEEVLNLAQSKNFHLRPRDLISNINVIVLELKGSETTFMCEYADETATIVEFLNR
WITFCQSIISTLT
SEQ ID NO: 6 (REH mutated IL-2 cytokine, human)
APTSSSTKKTQLQLEHLRLDLEMILNGINNYKNPKLTRMLTFKFYMPKKATELKHLQCL
EEELKPLEEVLNLAQSKNFHLRPRDLISNINVIVLELKGSETTFMCEYADETATIVEFLNR
WITFCHSIISTLT
SEQ ID NO: 7 (IL-2Rβ, human)
AVNGTSQFTCFYNSRANISCVWSQDGALQDTSCQVHAWPDRRRWNQTCELLPVSQAS
WACNLILGAPDSQKLTTVDIVTLRVLCREGVRWRVMAIQDFKPFENLRLMAPISLQVV
HVETHRCNISWEISQASHYFERHLEFEARTLSPGHTWEEAPLLTLKQKQEWICLETLTPD
TQYEFQVRVKPLQGEFTTWSPWSQPLAFRTKPAALGKDTIPWLGHLLVGLSGAFGFIIL
VYLLINCRNTGPWLKKVLKCNTPDPSKFFSQLSSEHGGDVQKWLSSPFPSSSFSPGGLAP
EISPLEVLERDKVTQLLLQQDKVPEPASLSSNHSLTSCFTNQGYFFFHLPDALEIEACQVY
FTYDPYSEEDPDEGVAGAPTGSSPQPLQPLSGEDDAYCTFPSRDDLLLFSPSLLGGPSPPS
TAPGGSGAGEERMPPSLQERVPRDWDPQPLGPPTPGVPDLVDFQPPPELVLREAGEEVP
DAGPREGVSFPWSRPPGQGEFRALNARLPLNTDAYLSLQELQGQDPTHLV
SEQ ID NO: 8 (Leader/signal sequence, human)
MLLLVTSLLLCELPHPAFLLIP
SEQ ID NO: 9 (Linker, artificial)
EQKLISEEDLEMETSQFPGEEKPQASPEGRPESETSC
SEQ ID NO: 10 (P2A, artificial)
SGATNFSLLKQAGDVEENPGP
SEQ ID NO: 11 (3x REH IL-2 tethered IL-2Rβ, human)
APTSSSTKKTQLQLEHLRLDLEMILNGINNYKNPKLTDMLTFEFYMPKKATELKHLQCL
ERELKPLEEVLNLAQSKNFHLRPRDLISRINVIVLELKGSETTFMCEYADETATIVEFLNR
WITFCHSIISTLTEQKLISEEDLEMETSQFPGEEKPQASPEGRPESETSCAVNGTSQFTCFY
NSRANISCVWSQDGALQDTSCQVHAWPDRRRWNQTCELLPVSQASWACNLILGAPDS
QKLTTVDIVTLRVLCREGVRWRVMAIQDFKPFENLRLMAPISLQVVHVETHRCNISWEI
SQASHYFERHLEFEARTLSPGHTWEEAPLLTLKQKQEWICLETLTPDTQYEFQVRVKPL
QGEFTTWSPWSQPLAFRTKPAALGKDTIPWLGHLLVGLSGAFGFIILVYLLINCRNTGP
WLKKVLKCNTPDPSKFFSQLSSEHGGDVQKWLSSPFPSSSFSPGGLAPEISPLEVLERDK
VTQLLLQQDKVPEPASLSSNHSLTSCFTNQGYFFFHLPDALEIEACQVYFTYDPYSEEDP
DEGVAGAPTGSSPQPLQPLSGEDDAYCTFPSRDDLLLFSPSLLGGPSPPSTAPGGSGAGE
ERMPPSLQERVPRDWDPQPLGPPTPGVPDLVDFQPPPELVLREAGEEVPDAGPREGVSF
PWSRPPGQGEFRALNARLPLNTDAYLSLQELQGQDPTHLV
SEQ ID NO: 12 (3x REH mutated IL-2 cytokine, human)
APTSSSTKKTQLQLEHLRLDLEMILNGINNYKNPKLTDMLTFEFYMPKKATELKHLQCL
ERELKPLEEVLNLAQSKNFHLRPRDLISRINVIVLELKGSETTFMCEYADETATIVEFLNR
WITFCHSIISTLT
SEQ ID NO: 13 (Extracellular domain of IL-2Rβ, human)
AVNGTSQFTCFYNSRANISCVWSQDGALQDTSCQVHAWPDRRRWNQTCELLPVSQAS
WACNLILGAPDSQKLTTVDIVTLRVLCREGVRWRVMAIQDFKPFENLRLMAPISLQVV
HVETHRCNISWEISQASHYFERHLEFEARTLSPGHTWEEAPLLTLKQKQEWICLETLTPD
TQYEFQVRVKPLQGEFTTWSPWSQPLAFRTKPAALGKDT
SEQ ID NO: 14 (Transmembrane domain of IL-2Rβ, human)
IPWLGHLLVGLSGAFGFIILVYLLI
SEQ ID NO: 15 (Intracellular domain of IL-2Rβ, human)
NCRNTGPWLKKVLKCNTPDPSKFFSQLSSEHGGDVQKWLSSPFPSSSFSPGGLAPEISPL
EVLERDKVTQLLLQQDKVPEPASLSSNHSLTSCFTNQGYFFFHLPDALEIEACQVYFTYD
PYSEEDPDEGVAGAPTGSSPQPLQPLSGEDDAYCTFPSRDDLLLFSPSLLGGPSPPSTAPG
GSGAGEERMPPSLQERVPRDWDPQPLGPPTPGVPDLVDFQPPPELVLREAGEEVPDAGP
REGVSFPWSRPPGQGEFRALNARLPLNTDAYLSLQELQGQDPTHLV
SEQ ID NO: 16 (Linker, artificial)
GGGGS
SEQ ID NO: 17 (Linker, artificial)
GGGGGGGGSGGGGS
SEQ ID NO: 18 (Linker, artificial)
SGGGSGGGGSGGGGSGGGGSGGGSLQ
SEQ ID NO: 19 (Linker, artificial)
GGGGSGGGGS
SEQ ID NO: 20 (Linker, artificial)
(GGGGS)ₙ
SEQ ID NO: 21 (Linker, artificial)
GSGPR

1. A regulatory T cell (Treg) comprising a recombinant cytokine receptor, wherein the recombinant cytokine receptor comprises:
an IL-2 cytokine;
an IL-2 receptor beta extracellular domain;
a transmembrane domain; and
an IL-2 receptor beta intracellular domain;
wherein the IL-2 receptor beta extracellular domain is tethered to the IL-2 cytokine by a polypeptide linker.

2. The Treg of claim 1, wherein the recombinant cytokine receptor forms a protein complex with IL-2Rγ.

3. The Treg of claim 1, wherein the recombinant cytokine receptor engages in IL-2 signaling in the absence of exogenous IL-2.

4. The Treg of claim 1, wherein the IL-2 cytokine comprises at least one amino acid substitution that reduces affinity for IL-2Rα and/or IL-2Rγ by at least about two-fold.

5. The Treg of claim 3, wherein the IL-2 cytokine comprises one or more amino acid substitutions selected from the amino acid positions 18, 22, 126, 38, 43, 61, 15, 16, 19, 20, 22, 23, and 81.

6. The Treg of claim 5, wherein the IL-2 cytokine comprises one or more amino acid substitutions selected from the group consisting of L18R, Q22E, Q126H, R38D, K43E, E61R, E15S, H16Q, L19V, D20L, Q22K, M23Q, M23A, and R81D.

7. The Treg of claim 6, wherein the IL-2 cytokine comprises the amino acid substitutions L18R, Q22E, and Q126H; and/or R38D, K43E, and E61R; and/or E15S, H16Q, L19V, D20L, M23Q, and R81D; and/or E15S, H16Q, L19V, D20L, Q22K, and M23A.

8. The Treg of claim 1, wherein the recombinant cytokine receptor comprises:
a. a WT IL-2 cytokine, a polypeptide linker, an IL-2Rβ extracellular domain, an IL-2Rβ transmembrane domain, and an IL-2Rβ intracellular domain;
b. a 3×IL-2 cytokine, a polypeptide linker, an IL-2Rβ extracellular domain, an IL-2Rβ transmembrane domain, and an IL-2Rβ intracellular domain;
c. an REH IL-2 cytokine, a polypeptide linker, an IL-2Rβ extracellular domain, an IL-2Rβ transmembrane domain, and an IL-2Rβ intracellular domain;
d. a 3×REH IL-2 cytokine, a polypeptide linker, an IL-2Rβ extracellular domain, an IL-2Rβ transmembrane domain, and an IL-2Rβ intracellular domain;
e. an IL-2 cytokine comprising substitutions at positions E15S, H16Q, L19V, D20L, M23Q, and R81D, a polypeptide linker, an IL-2Rβ extracellular domain comprising substitutions at positions H133D and Y134F, an IL-2Rβ transmembrane domain, and an IL-2Rβ intracellular domain; or
f. an IL-2 cytokine comprising substitutions at positions E15S, H16Q, L19V, D20L, Q22K, and M23A, a polypeptide linker, an IL-2Rβ extracellular domain comprising substitutions at positions H133D and Y134F, an IL-2Rβ transmembrane domain, and an IL-2Rβ intracellular domain.

9. The Treg of claim 1, wherein the polypeptide linker comprises glycines and serines.

10. The Treg of claim 8, wherein the recombinant cytokine receptor comprises:
a. an IL-2 cytokine comprising the amino acid sequence of SEQ ID NO:4, a polypeptide linker, an IL-2Rβ extracellular domain, an IL-2Rβ transmembrane domain, and an IL-2Rβ intracellular domain;
b. an IL-2 cytokine comprising the amino acid sequence of SEQ ID NO:5, a polypeptide linker, an IL-2Rβ extracellular domain, an IL-2Rβ transmembrane domain, and an IL-2Rβ intracellular domain;
c. an IL-2 cytokine comprising the amino acid sequence of SEQ ID NO:6, a polypeptide linker, an IL-2Rβ extracellular domain, an IL-2Rβ transmembrane domain, and an IL-2Rβ intracellular domain; or
d. an IL-2 cytokine comprising the amino acid sequence of SEQ ID NO:12, a polypeptide linker, an IL-2Rβ extracellular domain, an IL-2Rβ transmembrane domain, and an IL-2Rβ intracellular domain.

11. The Treg of claim 1, wherein the recombinant cytokine receptor comprises:
a. an IL-2 cytokine comprising an amino acid sequence comprising at least 80%, at least 85%, at least 90%, at least 95%, at least 96%, at least 97%, at least 98%, at least 99% or 100% sequence identity with the amino acid sequence selected from the group consisting of SEQ ID NOs:4-6 and 12;
b. a polypeptide linker comprising an amino acid sequence selected from the group consisting of SEQ ID NOs:9 and 16-21;
c. an IL-2 receptor beta extracellular domain comprising at least 80%, at least 85%, at least 90%, at least 95%, at least 96%, at least 97%, at least 98%, at least 99% or 100% sequence identity with the amino acid sequence set forth in SEQ ID NO:13;
d. an IL-2 receptor beta transmembrane domain comprising at least 80%, at least 85%, at least 90%, at least 95%, at least 96%, at least 97%, at least 98%, at least 99% or 100% sequence identity with the amino acid sequence set forth in SEQ ID NO:14; and/or
e. an IL-2 receptor beta intracellular domain comprises at least 80%, at least 85%, at least 90%, at least 95%, at least 96%, at least 97%, at least 98%, at least 99% or 100% sequence identity with the amino acid sequence set forth in SEQ ID NO:15.

12. The Treg of claim 11, wherein the IL-2 cytokine comprises an amino acid sequence comprising at least 80%, at least 85%, at least 90%, at least 95%, at least 96%, at least 97%, at least 98%, at least 99% or 100% sequence identity with the amino acid sequence set forth in SEQ ID NO:4.

13. The Treg of claim 12, wherein the recombinant cytokine receptor does not comprise a T cell receptor activation domain or a T cell costimulatory domain, optionally wherein the T cell receptor activation domain or T cell costimulatory domain is a CD28 signaling domain.

14. The Treg of claim 1, comprising:
a. an IL-2 cytokine comprising the amino acid sequence of SEQ ID NO:4, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15;
b. an IL-2 cytokine comprising the amino acid sequence of SEQ ID NO:5, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15;
c. an IL-2 cytokine comprising the amino acid sequence of SEQ ID NO:6, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15; or
d. an IL-2 cytokine comprising the amino acid sequence of SEQ ID NO:12, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15.

15. The Treg of claim 14, wherein the recombinant cytokine receptor comprises an amino acid sequence comprising at least 80%, at least 85%, at least 90%, at least 95%, at least 96%, at least 97%, at least 98%, at least 99% or 100% sequence identity with the amino acid sequence selected from the group consisting of SEQ ID NOs:1-3 and 11.

16. The Treg of claim 1, comprising a recombinant cytokine receptor consisting of:
an IL-2 cytokine;
an IL-2 receptor beta extracellular domain;
a transmembrane domain; and
an IL-2 receptor beta intracellular domain;
wherein the IL-2 receptor beta extracellular domain is tethered to the IL-2 cytokine thereof by a polypeptide linker.

17. The Treg of claim 1, wherein the Treg is CD25+ and expresses FOXP3 and/or HELIOS.

18. The Treg of claim 17, further comprising a chimeric antigen receptor (CAR).

19. A method of treating an immune-related disorder in an individual comprising administering the Treg of claim 1 to the individual.

20. The method of claim 19, wherein the cells are autologous to the individual.

21. The method of claim 19, wherein the individual is human.

22. The method of claim 19, wherein the Treg prevents, ameliorates, or cures an immune-related disorder.

23. A recombinant cytokine receptor comprising:
an IL-2 cytokine;
an IL-2 receptor beta extracellular domain;
a transmembrane domain; and
an IL-2 receptor beta intracellular domain,
wherein the IL-2 receptor beta extracellular domain is tethered to the IL-2 cytokine thereof by a polypeptide linker.

24. The recombinant cytokine receptor of claim 23, wherein the recombinant cytokine receptor forms a protein complex with IL-2Rγ.

25. The recombinant cytokine receptor of claim 23, wherein the recombinant cytokine receptor engages in IL-2 signaling in the absence of exogenous IL-2.

26. The recombinant cytokine receptor of claim 23, wherein the IL-2 cytokine comprises at least one amino acid substitution that reduces affinity for IL-2Rα and/or IL-2Rγ by at least about two-fold.

27. The recombinant cytokine receptor of claim 26, wherein the IL-2 cytokine comprises one or more amino acid substitutions selected from the amino acid positions 18, 22, 126, 38, 43, 61, 15, 16, 19, 20, 22, 23, and 81.

28. The recombinant cytokine receptor of claim 27, wherein the IL-2 cytokine comprises one or more amino acid substitutions selected from the group consisting of L18R, Q22E, Q126H, R38D, K43E, E61R, E15S, H16Q, L19V, D20L, Q22K, M23Q, M23A, and R81D.

29. The recombinant cytokine receptor of claim 28, wherein the IL-2 cytokine comprises the amino acid substitutions L18R, Q22E, and Q126H; and/or R38D, K43E, and E61R; and/or E15S, H16Q, L19V, D20L, M23Q, and R81D; and/or E15S, H16Q, L19V, D20L, Q22K, and M23A.

30. The recombinant cytokine receptor of claim 23, comprising:
a. a WT IL-2 cytokine, a polypeptide linker, an IL-2Rβ extracellular domain, an IL-2Rβ transmembrane domain, and an IL-2Rβ intracellular domain;
b. a 3×IL-2 cytokine, a polypeptide linker, an IL-2Rβ extracellular domain, an IL-2Rβ transmembrane domain, and an IL-2Rβ intracellular domain;
c. an REH IL-2 cytokine, a polypeptide linker, an IL-2Rβ extracellular domain, an IL-2Rβ transmembrane domain, and an IL-2Rβ intracellular domain;
d. a 3×REH IL-2 cytokine, a polypeptide linker, an IL-2Rβ extracellular domain, an IL-2Rβ transmembrane domain, and an IL-2Rβ intracellular domain;
e. an IL-2 cytokine comprising substitutions at positions E15S, H16Q, L19V, D20L, M23Q, and R81D, a polypeptide linker, an IL-2Rβ extracellular domain comprising substitutions at positions H133D and Y134F, an IL-2Rβ transmembrane domain, and an IL-2Rβ intracellular domain; or
f. an IL-2 cytokine comprising substitutions at positions E15S, H16Q, L19V, D20L, Q22K, and M23A, a polypeptide linker, an IL-2Rβ extracellular domain comprising substitutions at positions H133D and Y134F, an IL-2Rβ transmembrane domain, and an IL-2Rβ intracellular domain.

31. The recombinant cytokine receptor of claim 30, comprising:
a. an IL-2 cytokine comprising the amino acid sequence of SEQ ID NO:4, a polypeptide linker, an IL-2Rβ extracellular domain, an IL-2Rβ transmembrane domain, and an IL-2Rβ intracellular domain;
b. an IL-2 cytokine comprising the amino acid sequence of SEQ ID NO:5, a polypeptide linker, an IL-2Rβ extracellular domain, an IL-2Rβ transmembrane domain, and an IL-2Rβ intracellular domain;
c. an IL-2 cytokine comprising the amino acid sequence of SEQ ID NO:6, a polypeptide linker, an IL-2Rβ extracellular domain, an IL-2R transmembrane domain, and an IL-2Rβ intracellular domain; or
d. an IL-2 cytokine comprising the amino acid sequence of SEQ ID NO:12, a polypeptide linker, an IL-2Rβ extracellular domain, an IL-2Rβ transmembrane domain, and an IL-2Rβ intracellular domain.

32. The recombinant cytokine receptor of claim 23, wherein the polypeptide linker comprises glycines and serines.

33. The recombinant cytokine receptor of claim 23, comprising:
a. an IL-2 cytokine comprising an amino acid sequence comprising at least 80%, at least 85%, at least 90%, at least 95%, at least 96%, at least 97%, at least 98%, at least 99% or 100% sequence identity with the amino acid sequence selected from the group consisting of SEQ ID NOs:4-6 and 12;
b. a polypeptide linker comprising an amino acid sequence selected from the group consisting of SEQ ID NOs:9 and 16-21;
c. an IL-2 receptor beta extracellular domain comprising at least 80%, at least 85%, at least 90%, at least 95%, at least 96%, at least 97%, at least 98%, at least 99% or 100% sequence identity with the amino acid sequence set forth in SEQ ID NO:13;
d. an IL-2 receptor beta transmembrane domain comprising at least 80%, at least 85%, at least 90%, at least 95%, at least 96%, at least 97%, at least 98%, at least 99% or 100% sequence identity with the amino acid sequence set forth in SEQ ID NO:14; and/or
e. an IL-2 receptor beta intracellular domain comprises at least 80%, at least 85%, at least 90%, at least 95%, at least 96%, at least 97%, at least 98%, at least 99% or 100% sequence identity with the amino acid sequence set forth in SEQ ID NO:15.

34. The recombinant cytokine receptor of claim 33, wherein the IL-2 cytokine comprises an amino acid sequence comprising at least 80%, at least 85%, at least 90%, at least 95%, at least 96%, at least 97%, at least 98%, at least 99% or 100% sequence identity with the amino acid sequence set forth in SEQ ID NO:4.

35. The recombinant cytokine receptor of claim 34, wherein the recombinant cytokine receptor does not comprise a T cell receptor activation domain or a T cell costimulatory domain, optionally wherein the T cell receptor activation domain or T cell costimulatory domain is a CD28 signaling domain.

36. The recombinant cytokine receptor of claim 33, comprising:
a. an IL-2 cytokine comprising the amino acid sequence of SEQ ID NO:4, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15;
b. an IL-2 cytokine comprising the amino acid sequence of SEQ ID NO:5, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15;
c. an IL-2 cytokine comprising the amino acid sequence of SEQ ID NO:6, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15; or
d. an IL-2 cytokine comprising the amino acid sequence of SEQ ID NO:12, a polypeptide linker comprising the amino acid sequence of SEQ ID NO:9, an IL-2Rβ extracellular domain comprising the amino acid sequence of SEQ ID NO:13, an IL-2Rβ transmembrane domain comprising the amino acid sequence of SEQ ID NO:14, and an IL-2Rβ intracellular domain comprising the amino acid sequence of SEQ ID NO:15.

37. The recombinant cytokine receptor of claim 36, wherein the recombinant cytokine receptor comprises an amino acid sequence comprising at least 80%, at least 85%, at least 90%, at least 95%, at least 96%, at least 97%, at least 98%, at least 99% or 100% sequence identity with the amino acid sequence selected from the group consisting of SEQ ID NOs:1-3 and 11.

38. The recombinant cytokine receptor of claim 23, consisting of:
an IL-2 cytokine;
an IL-2 receptor beta extracellular domain;
a transmembrane domain; and
an IL-2 receptor beta intracellular domain;
wherein the IL-2 receptor beta extracellular domain is tethered to the IL-2 cytokine thereof by a polypeptide linker.

39. A nucleic acid encoding the recombinant cytokine receptor of claim 23.

40. A vector comprising the nucleic acid of claim 39.

41. The vector of claim 40, wherein the vector is a lentiviral vector.

42. The vector of claim 40, further comprising a marker gene.

43. The vector of claim 42, wherein the marker gene is a transmembrane protein.

44. The vector of claim 43, wherein the transmembrane protein is EGFR.

45. A composition comprising the Treg of claim 1, the recombinant cytokine receptor of claim 23, the nucleic acid of claim 39, or the vector of claim 40.

46. A method of expanding a transduced Treg cell in the absence of exogenous IL-2 comprising introducing the nucleic acid of claim 39 or the vector of claim 40 into the Treg cell and culturing the cell.

47. The method of claim 46, wherein the recombinant cytokine receptor
i) does not activate IL-2 signaling on a cell that does not comprise the recombinant cytokine receptor,
ii) does not activate IL-2 signaling on Tregs that do not comprise the recombinant cytokine receptor, and/or
iii) does not activate signaling of an IL-2 receptor comprising a different amino acid sequence.

48. The method of claim 46, where the recombinant cytokine receptor does not cause the transduced Treg cell to secrete one or more cytokines at higher levels than a Treg cell cultured with exogenous IL-2 cytokine.

49. The method of claim 46, further comprising detecting at least one Treg marker selected from the group consisting of CD4+, CD25+, and CD127lo, optionally further comprising detecting FOXP3 and/or HELIOS.

50. The method of claim 46, further comprising detecting methylation at Treg-specific differentially regulated genes.

51. The method of claim 50, wherein the Treg-specific differentially regulated gene is FOXP3.

52. The method of claim 46, wherein the Treg cell is able to proliferate in the absence of IL-2.

53. The method of claim 46, wherein one or more markers of endogenous IL-2 signaling is detected, optionally wherein the one or more markers of endogenous IL-2 signaling comprises phosphorylated STAT-5.

54. The method of claim 46, wherein the method generates Treg cells comprising a recombinant cytokine receptor, wherein:
i. the in vitro and/or in vivo suppressive activity of the Treg on CD8+ and/or CD4+ T cells is increased compared to a control Treg that is not transduced with the recombinant cytokine receptor,
ii. the rate of division of CD4+ and/or CD8+ T cells is decreased when cultured in the presence of the Treg compared to the rate of division of CD4+ and/or CD8+ T cells when cultured without the Treg;
iii. the relative amount of Treg cells in a composition comprising a population of Treg cells transduced with a recombinant cytokine receptor increases over time;
iv. a composition comprising a population of Treg cells transduced with the recombinant cytokine receptor grown without IL-2 contains a similar number of viable cells compared to a composition comprising a population of the same Treg cells not transduced with the recombinant cytokine receptor grown with IL-2;
v. at least 80% of the cells in the population of Treg cells transduced with the recombinant cytokine receptor maintain FOXP3 and/or HELIOS expression about 14 days after transduction;
vi. at least 80% of the cells in the population of Treg cells transduced with the recombinant cytokine receptor maintain FOXP3 and/or HELIOS expression about 23 days after transduction;
vii. the population of Treg cells transduced with the recombinant cytokine receptor expands at least two-fold more than a population of the same Treg cells not transduced with the recombinant cytokine receptor;
viii. the IL-10 cytokine levels produced by the Treg in vitro and/or in vivo are equivalent or increased compared to a control Treg that is not transduced with the recombinant cytokine receptor,
ix. the IFN-γ cytokine levels produced by the Treg in vitro and/or in vivo are equivalent or increased compared to a control Treg that is not transduced with the recombinant cytokine receptor,
x. the Gr-B cytokine levels produced by the Treg in vitro and/or in vivo are equivalent or increased compared to a control Treg that is not transduced with the recombinant cytokine receptor,
xi. the Treg functionally re-activates to higher levels than control Tregs that are not transduced with the recombinant cytokine receptor in vitro and/or in vivo; and/or
xii. the level of Treg proliferation increases from re-stimulation in vitro and/or in vivo one or more times.

55. The method of claim 46, further comprising culturing the Treg cell in a composition comprising a cytokine other than IL-2.

56. The method of claim 46, wherein the population of Treg cells transduced with the recombinant cytokine receptor expands at least two-fold, and wherein the population of Treg cells transduced with the recombinant cytokine receptor maintains expression of least one Treg marker selected from the group consisting of CD25+, FOXP3, and HELIOS.

57. The method of claim 19, wherein the individual expresses IL-2.
